(19) United States
(12) Patent Application Publication
(10) Pub. No.: US 2026/0209160 A1
(43) Pub. Date: Jul. 23, 2026

(54) METHOD FOR PRODUCING COMPOUND, POLYMER, COMPOSITION, AND PATTERN FORMATION METHOD

(71) Applicant: Mitsubishi Gas Chemical Company, Inc., Tokyo (JP)

(72) Inventors: Tadashi OMATSU, Hiratsuka-shi, Kanagawa (JP); Kentaro KATAOKA, Niigata-shi, Niigata (JP); Masahiro MATSUMOTO, Niigata-shi, Niigata (JP); Yushi NIIMI, Niigata-shi, Niigata (JP); Takeru KOGUMA, Hiratsuka-shi, Kanagawa (JP); Junya HORIUCHI, Kurashiki-shi, Okayama (JP); Hiroaki YAMAMOTO, Hiratsuka-shi, Kanagawa (JP); Masataka IINUMA, Niigata-shi, Niigata (JP); Kodai MATSUURA, Kurashiki-shi, Okayama (JP); Takashi SATO, Hiratsuka-shi, Kanagawa (JP); Yu OKADA, Chiyoda-ku, Tokyo (JP); Takashi MAKINOSHIMA, Hiratsuka-shi, Kanagawa (JP); Masatoshi ECHIGO, Chiyoda-ku, Tokyo (JP)

(21) Appl. No.: 19/140,657

(22) PCT Filed: Dec. 20, 2023
(86) PCT No.: PCT/JP2023/045591

§ 371 Date: Jun. 18, 2025

(30) Foreign Application Priority Data
Dec. 20, 2022 (JP) 2022-203721
Oct. 23, 2023 (JP) 2023-182078

(51) Int. Cl. C07C 67/08; C07C 41/18; C07C 47/575; C07C 51/09

(52) U.S. Cl. CPC C07C 67/08; C07C 41/18; C07C 47/575; C07C 51/09

(57) ABSTRACT

A method for producing a compound A represented by the following formula (1):

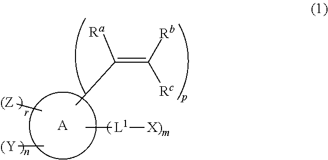

wherein each definition is as shown in the specification,
the method comprising at least one of step HX, step ST, and step PR:
step HX: a step of introducing a halogen or a group containing a halogen,
step ST: a step of introducing an unsaturated double bond, and
step PR: a step of introducing a group represented by the following formula (Y-0):

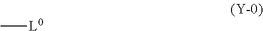

wherein each definition is as shown in the specification.

### TECHNICAL FIELD

The present invention relates to a method for producing a compound, a polymer, a composition, and a pattern formation method.

### BACKGROUND ART

In recent years, in the production of semiconductor elements and liquid crystal display elements, semiconductors (patterns) and pixels have been rapidly miniaturized due to the advance in lithography technology. For pixel miniaturization, the exposure light source has been shifted to have a shorter wavelength, in general. Specifically, ultraviolet rays typified by g-ray and i-ray have been used conventionally, but nowadays, an exposure method for using far ultraviolet such as KrF excimer laser (248 nm) and ArF excimer laser (193 nm) is being the center of mass production. Furthermore, the introduction of extreme ultraviolet (EUV) lithography (13.5 nm) is progressing. In addition, electron beam (EB) is also used for forming a fine pattern.

Up to now, typical resist materials are polymer based resist materials capable of forming an amorphous film. Examples include polymer based resist compositions such as polymethyl methacrylate, polyhydroxy styrene with an acid dissociation group, and polyalkyl methacrylate (see, for example, Non-Patent Literature 1). Conventionally, a line pattern of about 10 to 100 nm is formed by irradiating a resist thin film made by coating a substrate with a solution of these resist compositions with ultraviolet, far ultraviolet, electron beam, extreme ultraviolet or the like.

In addition, lithography using electron beam or extreme ultraviolet has a reaction mechanism different from that of normal photolithography (see Non-Patent Literature 2, Non-Patent Literature 3). Furthermore, lithography with electron beam or extreme ultraviolet aims at forming fine patterns of several nm to ten-odd nm. Accordingly, there is a demand for a resist composition having higher sensitivity to an exposure light source when the dimension of the resist pattern is reduced. In particular, lithography with extreme ultraviolet is required to further increase sensitivity in terms of throughput.

As the technology for improving these problems, various technnologies have been proposed (see, for example, Patent Literatures 1 to 11, Non-Patent Literature 4).

### CITATION LIST

### Patent Literature

Patent Literature 1: Japanese Patent Laid-Open No. 2015-108781
Patent Literature 2: US2019/0,187,342
Patent Literature 3: International Publication No. WO 2019/187881
Patent Literature 4: U.S. Pat. No. 4,316,995
Patent Literature 5: U.S. Pat. No. 5,274,060
Patent Literature 6: International Publication No. WO 2005/097719
Patent Literature 7: Japanese Patent Laid-Open No. 2021-188040
Patent Literature 8: Japanese Patent Laid-Open No. 2001-72614
Patent Literature 9: Japanese Patent Laid-Open No. 5-213779
Patent Literature 10: Japanese Patent Laid-Open No. 2014-218471
Patent Literature 11: Japanese Patent Laid-Open No. 2009-196963

### Non-Patent Literature

Non-Patent Literature 1: Shinji Okazaki, and eight others “Development of Lithography Technology in These 40 Years”, S&T Publishing Inc., Dec. 9, 2016
Non-Patent Literature 2: H. Yamamoto, et al., Jpn. J. Appl. Phys. 46, L142 (2007)
Non-Patent Literature 3: H. Yamamoto, et al., J. Vac. Sci. Technol. b 23, 2728 (2005)
Non-Patent Literature 4: J. Org. Chem. 2017, 82, 9931-9936

### SUMMARY OF INVENTION

### Technical Problem

As a resist material that solves the problems as mentioned above, a resist composition having a metallic complex such as titanium, tin, hafnium and zirconium has been proposed (see, for example, Patent Literature 1).

There is a demand for a resist composition having higher sensitivity to an exposure light source when the dimension of the resist pattern is reduced, and 4-hydroxystyrene containing iodine is proposed as a raw material monomer of the resist composition, (see, for example, Patent Literatures 2 to 3), but the method for synthesizing iodine-containing hydroxystyrene and an acetylated derivative thereof is not disclosed.

On the other hand, methods for synthesizing hydroxystyrene containing no iodine and an acetylated derivative thereof are widely known (see, for example, Patent Literatures 4 to 7). However, these methods require an expensive reagent and stringent conditions, and give a low yield and purity, in general. When these synthesis methods are applied to halogen-containing hydroxystyrene and a hydroxy group derivative thereof, the yield is further reduced in general, and the tendency thereof is particularly significant in iodine-containing hydroxystyrene and an acetylated derivative thereof.

As one of the methods for synthesizing hydroxystyrene, a method for introducing an unsaturated double bond utilizing a Wittig reaction is widely known in the art. The Wittig reaction on an aldehyde group or a ketone group contained in a raw material is likely to proceed quantitatively and is utilized in general at laboratory level, as a method for conveniently introducing an unsaturated double bond. On the other hand, since it is difficult to remove phosphine oxide that is produced after the Wittig reaction as a by-product in general, the purity is reduced, and thus, the Wittig reaction is a reaction still having a problem in the mass-production process.

As a method for removing phosphine oxide, a method in which sulfuric acid is added into a mixed solvent of alcohol and water and a compound having an unsaturated double bond is recovered by recrystallization is known (see, for example, Patent Literature 8). This method is an approach for separating the compound having an unsaturated double bond by recrystallization using a difference in solubility between phosphine oxide or a composite of phosphine oxide and sulfuric acid, and the compound having an unsaturated double bond. This approach has a problem in that the yield of the target compound having an unsaturated double bond is reduced upon certain removal of phosphine oxide.

As another method for removing phosphine oxide, a method for dissolving phosphine oxide in a lower fatty acid is known (see, for example, Patent Literature 9). This approach has a problem in that the viscosity of the lower fatty acid phase in which phosphine oxide is dissolved is high, so that it is difficult to discharge the lower phase from a reaction vessel for industrial use.

As a further method for removing phosphine oxide, a method for adding zinc chloride as a Lewis acid into an ethanol solvent is known (see, for example, Non-Patent Literature 4). In this approach, a polar solvent such as an alcohol-based or an ester-based one is used, so that an excess of zinc chloride is essential for certainly removing phosphine oxide. When the Wittig reaction generating a compound having an unsaturated double bond is used in combination, the above method has a problem in that an excess Lewis acid induces polymerization of the compound having an unsaturated double bond, leading to a reduction in yield and purity. In addition, the resist composition prepared using this approach has a problem in that metal atoms derived from zinc chloride used are contaminated, resulting in defects on a resist pattern.

As another one of the methods for synthesizing hydroxystyrene, a method for introducing an unsaturated double bond by a decarboxylation reaction through a cinnamic acid structure using aldehyde or ketone as a raw material is known (see, for example, Patent Literature 10). As another method, a method for introducing an unsaturated double bond by a dehydration reaction from a raw material having an alcohol group or an alkoxide group is known (see, for example, Patent Literature 11). In these methods, a reaction temperature of about 150° C. is required to efficiently proceed decarboxylation. When this method has been used as the method for synthesizing a compound having an electron-withdrawing group such as an iodine-containing hydroxystyrene derivative, there has been a problem in that the polymerization reaction of the styrene moiety has proceeded by heat, so that the target compound has not been stably obtained.

As described above, the method for producing halogen-containing hydroxystyrene and a hydroxy group derivative thereof has problems in that an expensive reagent and severe conditions are required in general, and the yield and purity of the halogen-containing hydroxystyrene and the hydroxy group derivative thereof are low.

To solve these problems, an object of the present invention is to provide a method for producing halogen-containing hydroxystyrene and a hydroxy group derivative thereof in a high yield and a high purity, as well as a compound obtained by the production method, a polymer, a composition, a composition for film formation, and a resist pattern formation method using the composition for film formation.

### Solution to Problem

The present inventors have, as a result of devoted examinations, found out that a production method capable of obtaining halogen-containing hydroxystyrene and a hydroxy group derivative thereof in a high yield and a high purity, and reached the present invention.

More specifically, the present invention is as follows.

[1]

A method for producing a compound A represented by the following formula (1):

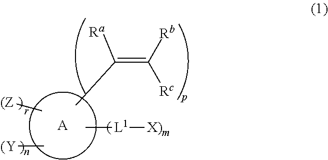

wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a urethane group, a urea group, an amide group, or an imide group, and the ether group, the ester group, the thioether group, the amino group, the thioester group, the acetal group, the urethane group, the urea group, the amide group, and the imide group of L¹ optionally have a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 18 carbon atoms and optionally having a substituent;
A is an organic group having 6 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
   p is an integer of 1 or more, m is an integer of 1 or more, n is an integer of 0 or more, and r is an integer of 0 or more;
the method comprising at least one of step HX, step ST, and step PR:
step HX: a step of introducing a halogen or a group containing a halogen,
step ST: a step of introducing an unsaturated double bond, and
step PR: a step of introducing a group represented by the following formula (Y-0):

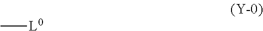

wherein each L⁰ is independently one or more groups selected from the group consisting of an alkoxy group, an ester group, an ether group, a thioether group, an acetal group, a thioacetal group, a carboxyalkoxy group, a carbonate ester group, a sulfonyl group, and a silyl group, or a hydrolyzable group.
   [2]

The production method according to [1], wherein the step ST comprises any one of step (W), step (C), and step (D):
step (W): a step comprising at least
   W1) a step of providing a carbonyl compound B represented by the following formula (3a) or the following formula (3b), and
   W2) a step of forming an unsaturated double bond from the carbonyl moiety of the carbonyl compound B:

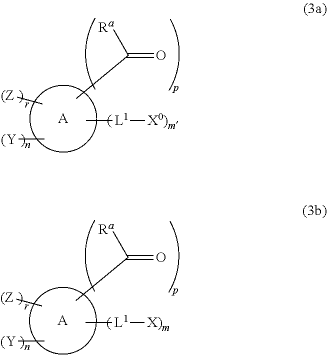

wherein
X, L¹, Y, R^{a}, Z, A, m, n, p, and r are as defined in the formula (1);
X⁰ is H or an organic group having 1 to 30 carbon atoms; and
m′ is an integer of 0 or more,
step (C): a step comprising at least
   C1) a step of providing the carbonyl compound B,
   C2) a step of obtaining a compound represented by the following formula (SA2a) or the following formula (SA2b) by using the carbonyl compound B and a compound represented by the formula (RM1) or malononitrile, and
   C3) a step of obtaining a compound represented by the following formula (0a) or the following formula (0b) by using the compound represented by the following formula (SA2a) or the following formula (SA2b) and a fluoride source:

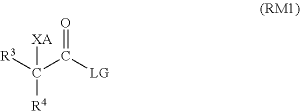

wherein
LG is a group selected from a hydroxy group, an alkoxy group, a carbonate ester group, an acetal group, and a carboxyl group, and the alkoxy group, the carbonate ester group, the acetal group, and the carboxyl group contain an aliphatic group or aromatic group having 1 to 60 carbon atoms and optionally having a substituent;
R³ is a hydrogen group, or a carboxyl group or ester group having 1 to 60 carbon atoms and optionally having a substituent;
R⁴ is a hydrogen group; and
XA is a group selected from a hydrogen group and a halogen group,

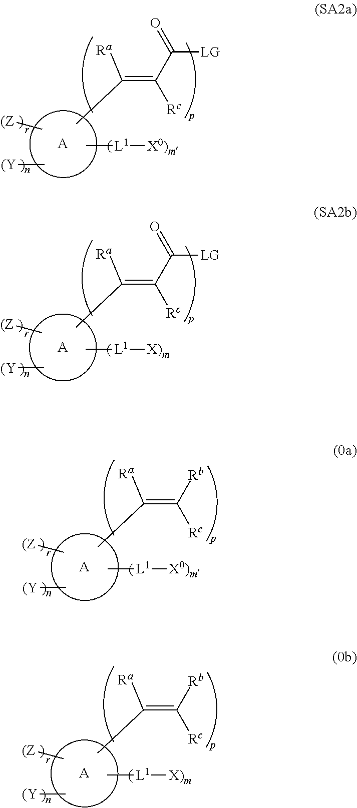

wherein
X, X⁰, L¹, Y, A, Z, p, m, m′, n, r, R^{a}, R^{b}, R^{c}, and LG are as defined in the formula (1), the formula (3a), the formula (3b), and the formula (RM1), and
step (D): a step comprising at least
   D1) a step of providing a compound represented by the formula (1-1a) or the formula (1-1b):

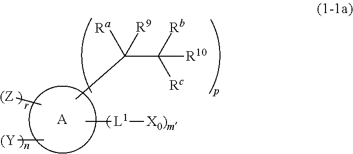

wherein
X₀, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{D} is an organic group having 1 to 30 carbon atoms and optionally having a substituent,

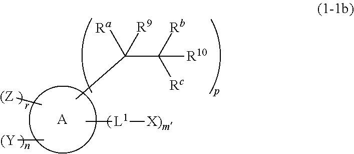

wherein
X, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{D} is an organic group having 1 to 30 carbon atoms and optionally having a substituent, and
   D2) a step of obtaining a compound represented by the following formula (0a) or the following formula (0b) from the compound represented by the formula (1-1a) or the formula (1-1b) by a dehydration reaction using an acid as a catalyst:

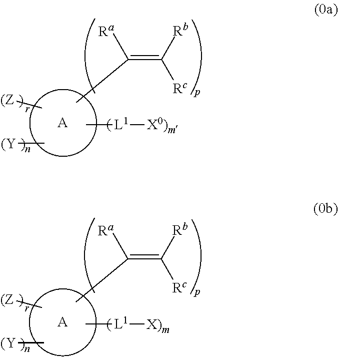

wherein
X, X⁰, L¹, Y, A, Z, p, m, m′, n, r, R^{a}, R^{b}, and R^{c} are as defined in the formula (1), the formula (3a), and the formula (3b).
   [3]

The production method according to [1] or [2], wherein an organic phosphorus compound is used in step W2.

[4]

The production method according to any one of [1] to [3], further comprising a step of solidifying phosphine oxide generated and removing the phosphine oxide by solid-liquid separation in step W2.

[5]

The production method according to any one of [1] to [4], wherein an acid is added to solidify the phosphine oxide as a composite with the acid.

[6]

The production method according to any one of [1] to [5], wherein amounts of substance of the acid and the phosphine oxide satisfy the following expression 1.

$$0.5 \leq \frac{\sum ( Amount of substance of the acid ) \times ( Valence of the acid )}{( Amount of substance of the phosphine oxide )} \leq 5$$

[7]

The production method according to any one of [1] to [6], wherein the phosphine oxide is solidified by changing a solvent system.

[8]

The production method according to any one of [1] to [7], wherein an organic base containing no alkali metal as a constituent element is further used in step W2.

[9]

The production method according to any one of [1] to [8], wherein
the carbonyl compound B is a compound represented by the following formula (4a) or the following formula (4b), and
an HSP distance between a compound represented by the following formula (5a) or the following formula (5b) formed by using an organic phosphorus compound in step W2 and the phosphine oxide is 6.5 or more:

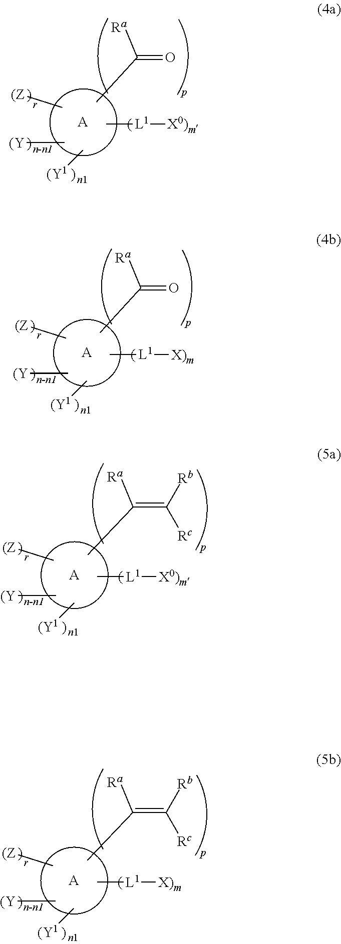

wherein
X, X⁰, L¹, Y, R^{a}, R^{b}, R^{c}, Z, A, m, m′, n, p, and r are as defined in the formula (1), the formula (3a), and the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally have a substituent; and
n1 is an integer of 1 or more and n or less.
   [10]

The production method according to any one of [1] to [9], wherein the step HX further comprises a step of halogenating the compound represented by the formula (3a) to produce the compound represented by the formula (3b), a step of halogenating the compound represented by the formula (0a) to produce the compound represented by the formula (0b), and a step of halogenating the compound represented by the formula (4a) to produce the compound represented by the formula (4b).

[11]

The production method according to any one of [1] to [10], wherein one or more solvents selected from the group consisting of γbutyrolactone, dimethylformamide, dioxane, cyclopentyl methyl ether, toluene, and diglyme are further used in step C3.

[12]

The production method according to any one of [1] to [11], wherein one or more solvents selected from the group consisting of polar aprotic solvents having a relative permittivity at 20° C. to 30° C. of 20 or more, and one or more solvents selected from the group consisting of ethanol, propanol, isopropyl alcohol, butanol, isobutanol, sec-butanol, tert-butanol, 1,2-dimethoxyethane, diisopropyl ether, ethyl acetate, tetrahydrofuran, dioxane, methyl ethyl ketone, carbon tetrachloride, chloroform, dichloroethane, benzene, toluene, o-xylene, cyclohexane, hexane, acetonitrile, nitromethane, and pyridine are further used in step D2.

[13]

The production method according to any one of [1] to [12], further comprising a step of removing impurities using an adsorbent.

[14]

The production method according to [1], further comprising a step of removing impurities by a reslurry treatment.

[15]

The production method according to [2], comprising step STPR of carrying out one step of step ST and step PR and then continuously carrying out the other step without carrying out isolation and purification operation.

[16]

The production method according to [15], wherein step ST is step (V).

[17]

The production method according to [16], wherein an organic phosphorus compound is used in step W2.

[18]

The production method according to [17], further comprising a step of solidifying phosphine oxide generated and removing the phosphine oxide by solid-liquid separation in step STPR.

[19]

The production method according to [18], wherein an acid is added to solidify the phosphine oxide as a composite with the acid.

[20]

The production method according to [19], wherein amounts of substance of the acid and the phosphine oxide satisfy the following expression 1.

$$0.5 \leq \frac{\sum ( Amount of substance of the acid ) \times ( Valence of the acid )}{( Amount of substance of the phosphine oxide )} \leq 5$$

The production method according to any one of [15] to [20], wherein step ST and step PR are carried out in the same reaction vessel.

[22]

The production method according to any one of [15] to [21], wherein one step is carried out and the other step is then continuously carried out without exchanging a solvent in step STPR.

[23]

A compound obtained by the production method according to any one of [1] to [22].

[24]

The method for producing a compound represented by the formula (3b) according to any one of [1] to [22], wherein the compound A represented by the formula (1) is a compound represented by the formula (3b), and the method comprises either step HX or step PR.

[25]

A compound obtained by the method for producing a compound according to [24].

[26]

The compound according to [25], wherein the compound represented by the formula (3b) is any of the following compounds.

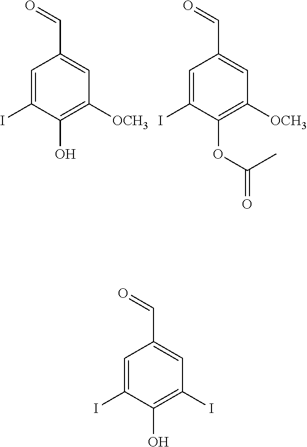

The production method according to [24], wherein the compound represented by the formula (3b) is represented by the following formula (4b):

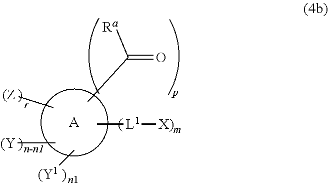

wherein
X, L¹, Y, R^{a}, Z, A, m, n, p, and r are as defined in the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally have a substituent; and
n1 is an integer of 1 or more and n or less.
   [28]

The production method according to [24] or [27], comprising a step of removing impurities using an adsorbent.

[29]

The production method according to [24], [27], or [28], further comprising a step of removing impurities by a reslurry treatment.

[30]

A polymer comprising a constitutional unit corresponding to the compound according to [23].

[31]

A composition comprising, at least, at least one selected from the group consisting of a compound obtained by the production method according to any one of [1] to [22], [24], and [27] to [29], and a polymer comprising a constitutional unit corresponding to the compound.

[32]

A composition for film formation comprising, at least, at least one selected from the group consisting of a compound obtained by the production method according to any one of [1] to [22], [24], and [27] to [29], and a polymer comprising a constitutional unit corresponding to the compound.

[33]

A resist pattern formation method comprising:
a step of forming a resist film on a substrate using the composition for film formation according to [32];
a step of exposing a pattern on the resist film; and
a step of subjecting the resist film after exposure to a development treatment.

### Advantageous Effects of Invention

The present invention can provide a production method capable of obtaining halogen-containing hydroxystyrene and a hydroxy group derivative thereof in a high yield and a high purity, as well as a compound obtained by the production method, a polymer, a composition, a composition for film formation, and a resist pattern formation method using the composition for film formation.

### BRIEF DESCRIPTION OF DRAWING

FIG. 1 is a flow chart of one example of the method for producing the compound A of the present embodiment.

### DESCRIPTION OF EMBODIMENTS

Hereinafter, embodiments to carry out the present invention will be described in more detail. Note that the present invention is not limited to the following embodiments and can be conducted with various modifications without departing from the spirit thereof.

As used herein, the meaning of each term is as follows.

The term “(meth)acrylate” means at least one selected from acrylate, haloacrylate, and methacrylate. The term haloacrylate means an acrylate in which the position of the methyl group in methacrylate is substituted with a halogen. Other terms having the expression (meth) should be similarly interpreted as (meth)acrylate.

The term “(co)polymer” means at least one selected from a homopolymer and a copolymer.

### <<Method for Producing Compound A>>

The method for producing the compound A of the present embodiment (hereinafter, may be referred to as “the production method of the present embodiment”) is a method for producing a compound A represented by the following formula (1):

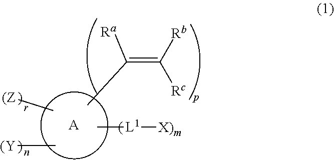

wherein
each X is independently I, F, Cl, Br, or an organic group having 1 to 30 carbon atoms and having 1 or more and 5 or less substituents selected from the group consisting of I, F, Cl, and Br;
   each L¹ is independently a single bond, an ether group, an ester group, a thioether group, an amino group, a thioester group, an acetal group, a urethane group, a urea group, an amide group, or an imide group, and the ether group, the ester group, the thioether group, the amino group, the thioester group, the acetal group, the urethane group, the urea group, the amide group, and the imide group of L¹ optionally have a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 18 carbon atoms and optionally having a substituent;
A is an organic group having 6 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and p is an integer of 1 or more, m is an integer of 1 or more, n is an integer of 0 or more, and r is an integer of 0 or more,
the method comprising at least one of step HX, step ST, and step PR:
step HX: a step of introducing a halogen or a group containing a halogen,
step ST: a step of introducing an unsaturated double bond, and
step PR: a step of introducing a group represented by the following formula (Y-0):

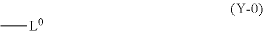

wherein each L⁰ is independently one or more groups selected from the group consisting of an alkoxy group, an ester group, an ether group, a thioether group, an acetal group, a thioacetal group, a carboxyalkoxy group, a carbonate ester group, a sulfonyl group, and a silyl group, or a hydrolyzable group.

### (Application of Compound A)

The compound A produced by the production method of the present embodiment can be used as a resist material because the polymer containing the compound A as a constitutional unit can increase the exposure sensibility of a resist composition.

### (Compound A)

The compound A in the present embodiment has one or more halogens and an unsaturated double bond. In addition, the compound A optionally further has one or more hydrophilic groups or one decomposable group. That is, the compound according to the present embodiment has one or more halogens, one or more hydrophilic groups or one decomposable group, and an unsaturated double bond. In addition, the compound A optionally further has one or more hydrophilic groups or one or more decomposable groups.

The term “hydrophilic group” means a group that is bonded to an organic compound, thereby improving the affinity between the organic compound and water. Examples of the hydrophilic group include a hydroxyl group, a nitro group, an amino group, a carboxyl group, a thiol group, a phosphine group, a phosphone group, a phosphate group, an ether group, a thioether group, a urethane group, a urea group, an amide group, and an imide group. Among them, from the viewpoint of improving the performance of a resist using the compound obtained by the present embodiment, in particular, from the viewpoint of developability, resolution, and reducing defects, a hydroxyl group or a carboxyl group is preferable, and a hydroxyl group is more preferable. The number of hydrophilic group is preferably an integer of 1 or more and 5 or less, more preferably an integer of 1 or more and 3 or less, further preferably 1 or 2, and particularly preferably 2. Note that, from the viewpoint of improving the performance of a resist using the compound obtained by the present embodiment, in particular, from the viewpoint of developability, resolution, and reducing defects, a structure in which a decomposable group is introduced and a structure after decomposition is a hydroxyl group or a carboxyl group can also preferably be used.

The term “decomposable group” means a group that is decomposed in the presence of an acid or a base, or by an action of irradiation from a light source such as radiation, electron beam, extreme ultraviolet (EUV), ArF, or KrF. The decomposable group is not particularly limited, and for example, an acid dissociable functional group described in International Publication No. WO 2013/024778 can be used. Among the decomposable groups, a hydrolyzable group is preferable. The term “hydrolyzable group” means a group that is hydrolyzed in the presence of an acid or a base. Examples of the hydrolyzable group include, but are not particularly limited to, an alkoxy group, an ester group, an acetal group, and a carbonate ester group. The number of decomposable group is not particularly limited, and is preferably an integer of 1 or more and 5 or less, more preferably an integer of 1 or more and 3 or less, further preferably 1 or 2, and particularly preferably 2.

The unsaturated double bond is preferably a polymerizable unsaturated double bond. Examples of the group having an unsaturated double bond include, but are not particularly limited to, a vinyl group, an isopropenyl group, a (meth)acryloyl group, and a haloacryloyl group. Examples of the haloacryloyl group include an α-fluoroacryloyl group, an α-chloroacryloyl group, an α-bromoacryloyl group, an α-iodoacryloyl group, an α,β-dichloroacryloyl group, and an α,β-diiodoacryloyl group. Among these unsaturated double bonds, an isopropenyl group or a vinyl group is preferable. The number of unsaturated double bond is preferably an integer of 1 or more and 3 or less, more preferably an integer of 1 or more and 2 or less, and further preferably 1.

A compound A of the present embodiment is a compound represented by the following formula (1). The compound A preferably contains a functional group for improving solubility in an alkaline developing solution by the action of an acid or a base, from the viewpoint of applications. The functional group for improving solubility in an alkaline developing solution by the action of an acid or a base is preferably contained in any of Z, Y, and X of the formula (1).

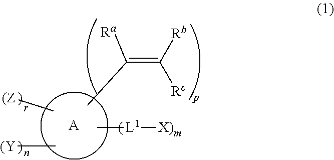

In the formula (1),
each X is independently I, F, Cl, Br, or an organic group having 1 to 30 carbon atoms and having 1 or more and 5 or less substituents selected from the group consisting of I, F, Cl, and Br. Among them, from the viewpoint of improving the number of functional group per unit volume of halogen elements (functional group density) and more efficiently exhibiting an effect of improving the sensibility of a resist, each X is preferably independently I, F, Cl, or Br, more preferably independently, I, F, or Br, further preferably independently, I or F, and particularly preferably independently I. The number of halogen is preferably an integer of 1 or more and 5 or less, more preferably an integer of 1 or more and 4 or less, and further preferably 1 or more and 3 or less.

In the present embodiment, unless otherwise defined, the term “substituted” means that one or more hydrogen atoms in a functional group are substituted with a substituent. Examples of the “substituent” include, but are not particularly limited to, a halogen atom, a hydroxyl group, a carboxyl group, a cyano group, a nitro group, a thiol group, a heterocyclic ring group, an alkyl group having 1 to 30 carbon atoms, an aryl group having 6 to 30 carbon atoms, an alkoxy group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms.

The alkyl group may be any of a linear aliphatic hydrocarbon group, a branched aliphatic hydrocarbon group, and a cyclic aliphatic hydrocarbon group.

Examples of the alkyl group having 1 to 30 carbon atoms include, but are not particularly limited to, a methyl group, an ethyl group, an n-propyl group, an i-propyl group, an n-butyl group, an i-butyl group, a t-butyl group, an n-pentyl group, an n-hexyl group, an n-dodecyl group, and a valeryl group.

Examples of the aryl group having 6 to 30 carbon atoms include, but are not particularly limited to, a phenyl group, a naphthalene group, a biphenyl group, an anthracyl group, a pyrenyl group, and a perylene group.

Examples of the alkenyl group having 2 to 30 carbon atoms include, but are not particularly limited to, an ethynyl group, a propenyl group, a butynyl group, and a pentynyl group.

Examples of the alkynyl group having 2 to 30 carbon atoms include, but are not particularly limited to, an acetylene group, an ethynyl group, and a propynyl group.

Examples of the alkoxy group having 1 to 30 carbon atoms include, but are not particularly limited to, a methoxy group, an ethoxy group, a propoxy group, a butoxy group, and pentoxy.

Examples of “the organic group having 1 to 30 carbon atoms and having 1 or more and 5 or less substituents selected from the group consisting of I, F, Cl, and Br” include, but are not particularly limited to, a monoiodophenyl group, a diiodophenyl group, a triiodophenyl group, a tetraiodophenyl group, a pentaiodophenyl group, a monoiodohydroxyphenyl group, a diiodohydroxyphenyl group, a triiodohydroxyphenyl group, a monoiodoacetoxyphenyl group, a diiodoacetoxyphenyl group, a triiodoacetoxyphenyl group, a monoiodo-t-butoxycarbonylphenyl group, a diiodo-t-butoxycarbonylphenyl group, a triiodo-t-butoxycarbonylphenyl group, a monoiododihydroxyphenyl group, a diiododihydroxyphenyl group, a triiododihydroxyphenyl group, a monoiododiacetoxyphenyl group, a diiododiacetoxyphenyl group, a triiododiacetoxyphenyl group, a monoiodo-di-t-butoxycarbonylphenyl group, a diiodo-di-t-butoxycarbonylphenyl group, a triiodo-di-t-butoxycarbonylphenyl group, a monoiodotrihydroxyphenyl group, a diiodotrihydroxyphenyl group, a monoiodotriacetoxyphenyl group, a diiodotriacetoxyphenyl group, a monoiodo-tri-t-butoxycarbonylphenyl group, a diiodo-tri-t-butoxycarbonylphenyl group, a monoiodonaphthyl group, a diiodonaphthyl group, a triiodonaphthyl group, a tetraiodonaphthyl group, a pentaiodonaphthyl group, a monoiodohydroxynaphthyl group, a diiodohydroxynaphthyl group, a triiodohydroxynaphthyl group, a monoiodoacetoxynaphthyl group, a diiodoacetoxynaphthyl group, a triiodoacetoxynaphthyl group, a monoiodo-t-butoxycarbonylnaphthyl group, a diiodo-t-butoxycarbonylnaphthyl group, a triiodo-t-butoxycarbonylnaphthyl group, a monoiododihydroxynaphthyl group, a diiododihydroxynaphthyl group, a triiododihydroxynaphthyl group, a monoiododiacetoxynaphthyl group, a diiododiacetoxynaphthyl group, a triiododiacetoxynaphthyl group, a monoiodo-di-t-butoxycarbonylnaphthyl group, a diiodo-di-t-butoxycarbonylnaphthyl group, a triiodo-di-t-butoxycarbonylnaphthyl group,
a monoiodotrihydroxynaphthyl group, a diiodotrihydroxynaphthyl group, a monoiodotriacetoxynaphthyl group, a diiodotriacetoxynaphthyl group, a monoiodo-tri-t-butoxycarbonylnaphthyl group, a diiodo-tri-t-butoxycarbonylnaphthyl group, a monoiodoadamantyl group, a diiodoadamantyl group, a triiodoadamantyl group, a monoiodohydroxyadamantyl group, a diiodohydroxynaphthyl group, a monoiodoacetoxynaphthyl group, a diiodoacetoxyadamantyl group, a monoiodo-t-butoxycarbonyladamantyl group, a diiodo-t-butoxycarbonyladamantyl group, a triiodo-t-butoxycarbonyladamantyl group, a monoiododihydroxyadamantyl group, a monoiododiacetoxyadamantyl group, a monoiodo-di-t-butoxycarbonyladamantyl group, a monoiodocyclohexyl group, a diiodocyclohexyl group, a triiodocyclohexyl group, a monoiodohydroxycyclohexyl group, a diiodohydroxynaphthyl group, a monoiodoacetoxynaphthyl group, a diiodoacetoxycyclohexyl group, a monoiodo-t-butoxycarbonylcyclohexyl group, a diiodo-t-butoxycarbonylcyclohexyl group, a triiodo-t-butoxycarbonylcyclohexyl group, a monoiododihydroxycyclohexyl group, a monoiododiacetoxycyclohexyl group, a monoiodo-di-t-butoxycarbonylcyclohexyl group,
a monobromophenyl group, a dibromophenyl group, a tribromophenyl group, a tetrabromophenyl group, a pentabromophenyl group, a monobromohydroxyphenyl group, a dibromohydroxyphenyl group, a tribromohydroxyphenyl group, a monobromoacetoxyphenyl group, a dibromoacetoxyphenyl group, a tribromoacetoxyphenyl group, a monobromo t-butoxycarbonylphenyl group, a dibromo t-butoxycarbonylphenyl group, a tribromo t-butoxycarbonylphenyl group, a monobromodihydroxyphenyl group, a dibromodihydroxyphenyl group, a tribromodihydroxyphenyl group, a monobromodiacetoxyphenyl group, a dibromodiacetoxyphenyl group, a tribromodiacetoxyphenyl group, a monobromo di-t-butoxycarbonylphenyl group, a dibromo di-t-butoxycarbonylphenyl group, a tribromo di-t-butoxycarbonylphenyl group,
a monobromotrihydroxyphenyl group, a dibromotrihydroxyphenyl group, a monobromotriacetoxyphenyl group, a dibromotriacetoxyphenyl group, a monobromotri-t-butoxycarbonylphenyl group, a dibromotri-t-butoxycarbonylphenyl group, a monobromoadamantyl group, a dibromoadamantyl group, a tribromoadamantyl group, a monobromohydroxyadamantyl group, a dibromohydroxynaphthyl group, a monobromoacetoxynaphthyl group, a dibromoacetoxyadamantyl group, a monobromo t-butoxycarbonyladamantyl group, a dibromo t-butoxycarbonyladamantyl group, a tribromo t-butoxycarbonyladamantyl group, a monobromodihydroxyadamantyl group, a monobromodiacetoxyadamantyl group, a monobromo-di-t-butoxycarbonyladamantyl group,
a monofluorophenyl group, a difluorophenyl group, a trifluorophenyl group, a tetrafluorophenyl group, a pentafluorophenyl group, a monofluorohydroxyphenyl group, a difluorohydroxyphenyl group, a trifluorohydroxyphenyl group, a monofluoroacetoxyphenyl group, a difluoroacetoxyphenyl group, a trifluoroacetoxyphenyl group, a monofluoro t-butoxycarbonylphenyl group, a difluoro t-butoxycarbonylphenyl group, a trifluoro t-butoxycarbonylphenyl group, a monofluorodihydroxyphenyl group, a difluorodihydroxyphenyl group, a trifluorodihydroxyphenyl group, a monofluorodiacetoxyphenyl group, a difluorodiacetoxyphenyl group, a trifluorodiacetoxyphenyl group, a monofluorodi-t-butoxycarbonylphenyl group, a difluorodi-t-butoxycarbonylphenyl group, a trifluorodi-t-butoxycarbonylphenyl group, a monofluorotrihydroxyphenyl group, a difluorotrihydroxyphenyl group, a monofluorotriacetoxyphenyl group, a difluorotriacetoxyphenyl group, a monofluorotri-t-butoxycarbonylphenyl group, a difluorotri-t-butoxycarbonylphenyl group, a monofluoroadamantyl group, a difluoroadamantyl group, a trifluoroadamantyl group, a monofluorohydroxyadamantyl group, a difluorohydroxynaphthyl group, a monofluoroacetoxynaphthyl group, a difluoroacetoxyadamantyl group, a monofluoro t-butoxycarbonyladamantyl group, a difluoro t-butoxycarbonyladamantyl group, a trifluoro t-butoxycarbonyladamantyl group, a monofluorodihydroxyadamantyl group, a monofluorodiacetoxyadamantyl group, a monofluoro-di-t-butoxycarbonyladamantyl group,
a monochlorophenyl group, a dichlorophenyl group, a trichlorophenyl group, a tetrachlorophenyl group, a pentachlorophenyl group, a monochlorohydroxyphenyl group, a dichlorohydroxyphenyl group, a trichlorohydroxyphenyl group, a monochloroacetoxyphenyl group, a dichloroacetoxyphenyl group, a trichloroacetoxyphenyl group, a monochloro t-butoxycarbonylphenyl group, a dichloro t-butoxycarbonylphenyl group, a trichloro t-butoxycarbonylphenyl group, a monochlorodihydroxyphenyl group, a dichlorodihydroxyphenyl group, a trichlorodihydroxyphenyl group, a monochlorodiacetoxyphenyl group, a dichlorodiacetoxyphenyl group, a trichlorodiacetoxyphenyl group, a monochlorodi-t-butoxycarbonylphenyl group, a dichlorodi-t-butoxycarbonylphenyl group, a trichlorodi-t-butoxycarbonylphenyl group,
a monochlorotrihydroxyphenyl group, a dichlorotrihydroxyphenyl group, a monochlorotriacetoxyphenyl group, a dichlorotriacetoxyphenyl group, a monochlorotri-t-butoxycarbonylphenyl group, a dichlorotri-t-butoxycarbonylphenyl group, a monochloroadamantyl group, a dichloroadamantyl group, a trichloroadamantyl group, a monochlorohydroxyadamantyl group, a dichlorohydroxynaphthyl group, a monochloroacetoxynaphthyl group, a dichloroacetoxyadamantyl group, a monochloro t-butoxycarbonyladamantyl group, a dichloro t-butoxycarbonyladamantyl group, a trichloro t-butoxycarbonyladamantyl group, a monochlorodihydroxyadamantyl group, a monochlorodiacetoxyadamantyl group, and a monochlorodi-t-butoxycarbonyladamantyl group.

X is not particularly limited, but may be an aromatic group into which one or more F, Cl, Br, or I are introduced. Examples of such an aromatic group include, but are not particularly limited to, a group having a benzene ring such as a phenyl group and having 1 to 5 halogens, and a group having a heteroaromatic ring such as furan, thiophene, and pyridine and having 1 to 5 halogens. Examples thereof include a phenyl group having 1 to 5 I, a phenyl group having 1 to 5 F, a phenyl group having 1 to 5 Cl, a phenyl group having 1 to 5 Br, a naphthyl group having 1 to 5 F, a naphthyl group having 1 to 5 Cl, a naphthyl group having 1 to 5 Br, a naphthyl group having 1 to 5 I, a phenol group having 1 to 4 F, a phenol group having 1 to 4 Cl, a phenol group having 1 to 4 Br, a phenol group having 1 to 4 I, a furan group having 1 to 3 F, a furan group having 1 to 3 Cl, a furan group having 1 to 3 Br, a furan group having 1 to 3 I, a thiophene group having 1 to 3 F, a thiophene group having 1 to 3 Cl, a thiophene group having 1 to 3 Br, a thiophene group having 1 to 3 I, a pyridine group having 1 to 4 F, a pyridine group having 1 to 4 Cl, a pyridine group having 1 to 4 Br, a pyridine group having 1 to 4 I, a benzodiazole group having 1 to 5 F, a benzodiazole group having 1 to 5 Cl, a benzodiazole group having 1 to 5 Br, a benzodiazole group having 1 to 5 I, a benzimidazole group having 1 to 4 F, a benzimidazole group having 1 to 4 Cl, a benzimidazole group having 1 to 4 Br, a benzimidazole group having 1 to 4 I, a benzoxazole group having 1 to 4 F, a benzoxazole group having 1 to 4 Cl, a benzoxazole group having 1 to 4 Br, a benzoxazole group having 1 to 4 I, a benzothiophene group having 1 to 4 F, a benzothiophene group having 1 to 4 Cl, a benzothiophene group having 1 to 4 Br, and a benzothiophene group having 1 to 4 I. X may be an alicyclic group into which one or more F, Cl, Br, or I are introduced. Examples of such an alicyclic group include an adamantyl group having 1 to 3 halogens, an adamantyl group having 1 to 3 F, an adamantyl group having 1 to 3 Cl, an adamantyl group having 1 to 3 Br, an adamantyl group having 1 to 3 I, a cyclopentyl group having 1 to 3 F, a cyclopentyl group having 1 to 3 Cl, a cyclopentyl group having 1 to 3 Br, a cyclopentyl group having 1 to 3 I, a bicycloundecyl group having 1 to 3 F, a bicycloundecyl group having 1 to 3 Cl, a bicycloundecyl group having 1 to 3 Br, a bicycloundecyl group having 1 to 3 I, a norbornyl group having 1 to 3 F, a norbornyl group having 1 to 3 Cl, a norbornyl group having 1 to 3 Br, and a norbornyl group having 1 to 3 I.

Each L¹ is independently a single bond, an ether group, an ester group, a thioether group, an amino group, a thioester group, an acetal group, a phosphine group, a phosphone group, a urethane group, a urea group, an amide group, an imide group, or a phosphate group. Among them, L¹ is preferably a single bond. The ether group, the ester group, the thioether group, the amino group, the thioester group, the acetal group, the phosphine group, the phosphone group, the urethane group, the urea group, the amide group, the imide group, or the phosphate group of L¹ optionally has a substituent. Examples of such a substituent include, but are not particularly limited to, the substituents as described above.

m is an integer of 1 or more, preferably an integer of 1 or more and 5 or less, more preferably an integer of 1 or more and 4 or less, and further preferably 1 or 2.

Each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent. Examples of such a substituent include, but are not particularly limited to, the substituents as described above.

Examples of Y include, but are not particularly limited to, at least one group selected from the group consisting of an alkoxy group [*³—O—R²], an ester group [*³—O—(C═O)—R² or *³—(C═O)—O—R²], an acetal group [*³—O—(C(R²¹)₂)—O—R² (wherein each R²¹ is independently H or a hydrocarbon group having 1 to 10 carbon atoms)], a carboxyalkoxy group [*³—O—R²²—(C═O)—O—R² (wherein R²² is a divalent hydrocarbon group having 1 to 10 carbon atoms)], and a carbonate ester group [*³—O—(C═O)—O—R²]. The ester group is preferably a tertiary ester group, from the viewpoint of achieving high exposure sensibility of a resist composition. In the formula, *³ is a site for binding with A.

Among them, Y is preferably a hydroxyl group, a tertiary ester group, an acetal group, a carbonate ester group, or a carboxyalkoxy group, more preferably a hydroxyl group, an acetal group, a carbonate ester group, or a carboxyalkoxy group, and further preferably a hydroxyl group, an acetal group, or a carboxyalkoxy group, from the viewpoint of achieving high exposure sensibility of a resist composition. In the production method of the compound A by the present invention, from the viewpoint of improving the stability in an acidic to weakly acidic process and from the viewpoint of producing a polymer having a stable quality by radical polymerization when the compound A is used as a resist material, Y is preferably an ester group, a carboxyalkoxy group, or a carbonate ester group, which functions as a protective group and is capable of forming a hydroxyl group or a carboxyl group preferred in terms of achieving high sensitivity of a resist composition, after deprotection.

Each Y is preferably independently a group represented by the following formula (Y-1).

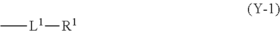

In the formula (Y-1),
L¹ is a group which is cleaved by the action of an acid or a base. Examples of the group which is cleaved by the action of an acid or a base include at least one divalent linking group selected from the group consisting of an ester group [*¹—O—(C═O)—*² or *¹—(C═O)—O—*²], an acetal group [*¹—O—(C(R²¹)₂)—O—*² (each R²¹ is independently H or a hydrocarbon group having 1 to 10 carbon atoms)], a carboxyalkoxy group [*¹—O—R²²—(C═O)—O—*² (R²² is a divalent hydrocarbon group having 1 to 10 carbon atoms)], and a carbonate ester group [*¹—O—(C═O)—O—*²]. The ester group is preferably a tertiary ester group, from the viewpoint of achieving high exposure sensibility of a resist composition. In the formula, *¹ is a site for binding with A, and *² is a site for binding with R¹. Among them, L¹ is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of achieving high exposure sensibility of a resist composition. In the production method of the compound A by the present invention, from the viewpoint of improving the stability in an acidic to weakly acidic process and from the viewpoint of producing a polymer having a stable quality by radical polymerization when the compound A is used as a resist material, L¹ is preferably an ester group, a carboxyalkoxy group, or a carbonate ester group.

As another effect, Y is preferably a group represented by the formula (Y-1) to control the polymerization properties of resin and the degree of polymerization in a desired range, when the compound A of the present embodiment is used as a polymerization unit of a copolymer. Since the compound A has a large influence on activity species in the polymer formation reaction due to having an X group and thus the desired control is difficult, variation of copolymer formation derived from the hydrophilic group and polymerization inhibition can be suppressed by having a group represented by the formula (Y-1) in the hydrophilic group in the compound A.

R¹ is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R¹ optionally further have a substituent. As the substituent here, the aforementioned substituents are used, but a linear, branched, or cyclic aliphatic group having 1 to 20 carbon atoms or an aromatic group having 6 to 20 carbon atoms are preferable. Among them, R¹ is preferably an aliphatic group. The aliphatic group in R¹ is preferably a branched or cyclic aliphatic group. The number of carbon atoms of aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L¹ is preferably *¹—(C═O)—O—*² or a carboxyalkoxy group, because, when L¹ is cleaved by the action of an acid or a base, a carboxylic acid group is formed, and when the compound A is used as a resist material, the difference in the solubility and the difference in the dissolution rate between a cleaved portion and an uncleaved portion are increased in the development treatment, so that the resolution is improved and, in particular, residues at the pattern bottom in thin line patterns are suppressed.

In the formula (Y-1), L¹ is not particularly limited, and is preferably, for example, a group represented by any of the following formulas.

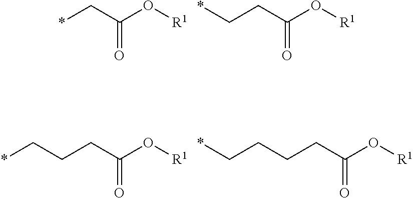

In the formula (Y-1), R¹ is not particularly limited, and is preferably, for example, a group represented by any of the following formulas.

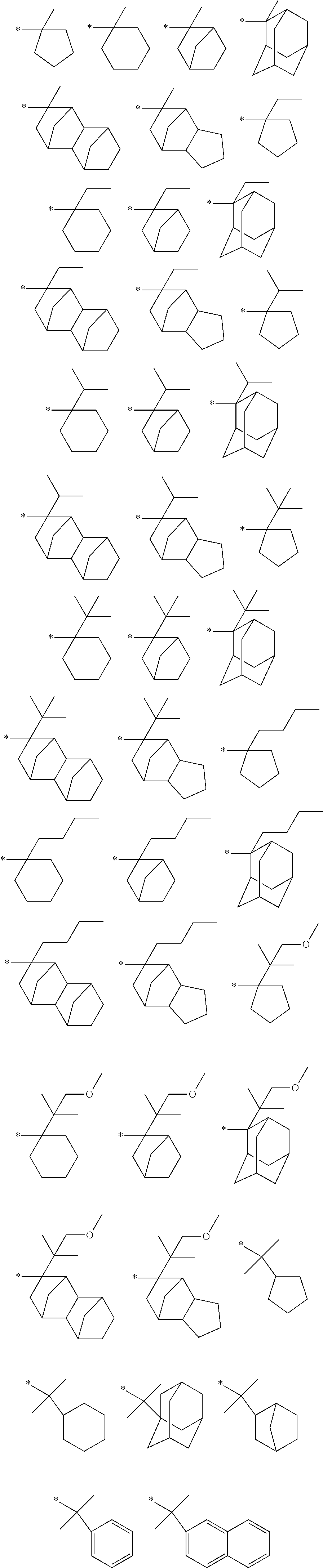

Y is not particularly limited, and is preferably, for example, a group represented by any of the following formulas. Further, examples of preferred groups as Y include *—O—C(═O)—CH₃ and *—O—CH(CH₃)—O—C₂H₅.

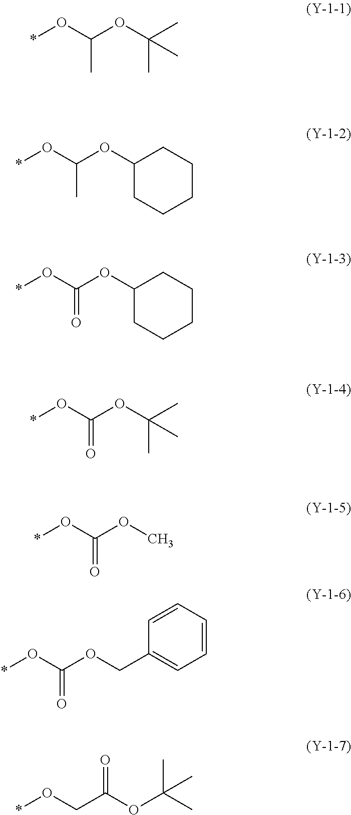

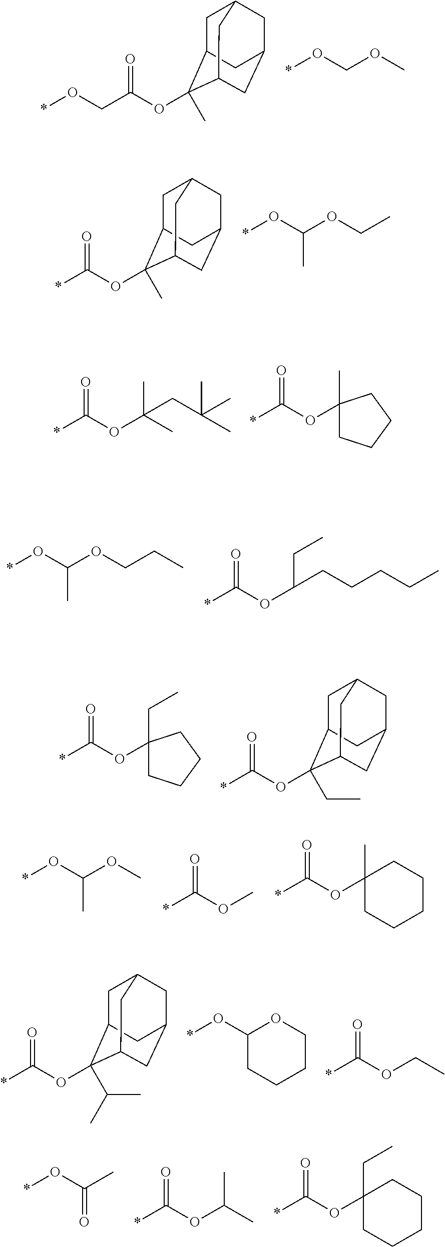

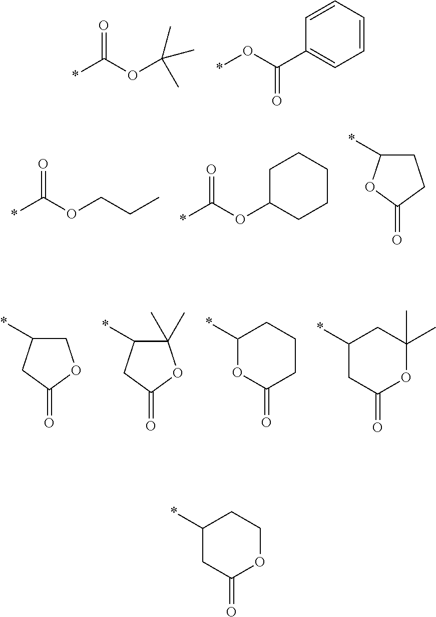

Examples of the alkoxy group that can be used as Y include an alkoxy group having 1 or more carbon atoms, and when the compound A is used as a resist material, from the viewpoint of the solubility of a resin after the compound is combined with other monomers to form the resin, an alkoxy group having 2 or more carbon atoms is preferable, and an alkoxy group having 3 or more carbon atoms or having a cyclic structure is more preferable. Examples of the alkoxy group include, but are not particularly limited to, the followings.

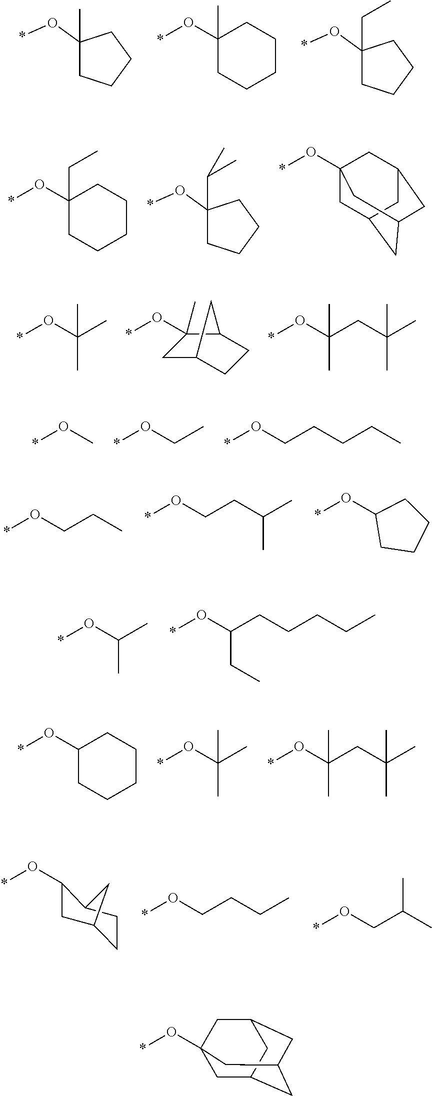

The amino group or amide group that can be used as Y is preferably a primary amino group, a secondary amino group, a tertiary amino group, a group having a quaternary ammonium salt structure, an amide having a substituent, or the like. Examples of the amino group or amide group include, but are not particularly limited to, the followings.

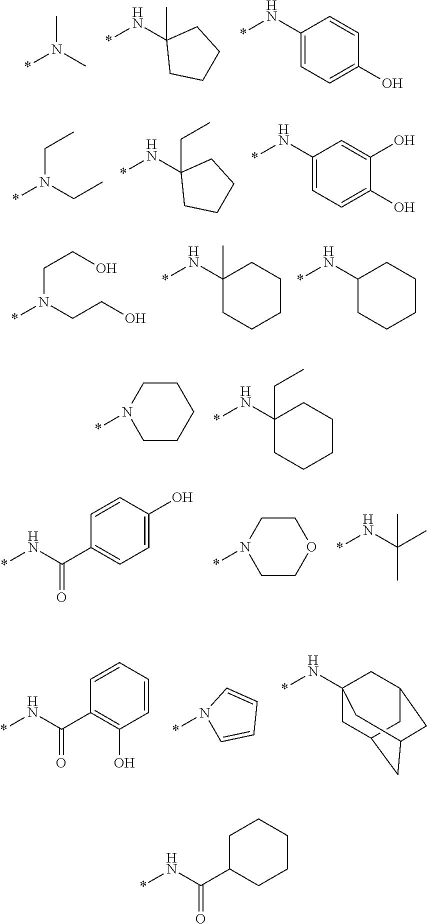

n is an integer of 0 or more, preferably an integer of 1 or more, more preferably an integer of 1 or more and 5 or less, further preferably an integer of 1 or more and 3 or less, and particularly preferably 1 or 2.

Each of R^{a}, R^{b}, and R^{c} is independently H, I, F, Cl, Br, or an organic group having 1 to 18 carbon atoms and optionally having a substituent. Examples of the substituent of the organic group having 1 to 18 carbon atoms include, but are not particularly limited to, I, F, Cl, Br, or other substituents. Examples of other substituents include, but are not particularly limited to, a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, and a phosphate group. Among them, the alkoxy group, the ester group, the carbonate ester group, the amino group, the ether group, the thioether group, the phosphine group, the phosphone group, the urethane group, the urea group, the amide group, the imide group, and the phosphate group optionally further have a substituent. Examples of the substituent here include a linear, branched, or cyclic aliphatic group having 1 to 20 carbon atoms and an aromatic group having 6 to 20 carbon atoms. With respect to R^{a}, R^{b}, and R^{c}, each of R^{a}, R^{b}, and R^{c} is preferably H (hydrogen).

The number of carbon atoms of the organic group optionally having a substituent in R^{a}, R^{b}, and R^{c} is preferably 1 to 8.

Examples of the organic group having 1 to 8 carbon atoms and optionally having a substituent include, but are not particularly limited to, a linear or branched aliphatic hydrocarbon group having 1 to 18 carbon atoms, a cycloaliphatic hydrocarbon group having 1 to 18 carbon atoms, and an aromatic group having 1 to 18 carbon atoms and optionally having a heteroatom.

Examples of the linear or branched aliphatic hydrocarbon group having 1 to 18 carbon atoms include, but are not particularly limited to, a methyl group, an ethyl group, an n-propyl group, an isopropyl group, an n-butyl group, an isobutyl group, a tert-butyl group, an n-pentyl group, and an n-hexyl group.

Examples of the cycloaliphatic hydrocarbon group having 1 to 18 carbon atoms include, but are not particularly limited to, a cyclohexyl group. Further, an aromatic group optionally having a heteroatom such as a benzodiazole group, a benzotriazole group, and a benzothiadiazole group can be arbitrarily selected. A combination of these organic groups can also be selected.

Examples of the aromatic group having 1 to 18 carbon atoms and optionally having a heteroatom include, but are not particularly limited to, a phenyl group, a naphthalene group, a biphenyl group, an anthracyl group, a pyrenyl group, a benzodiazole group, a benzotriazole group, and a benzothiadiazole group.

Among these organic groups having 1 to 18 carbon atoms and optionally having a substituent, a methyl group is preferable, but is not particularly limited, from the viewpoint of producing a polymer having a stable quality when the compound A is used as a resist material.

When R^{a} is an organic group having 1 to 18 carbon atoms, or a group selected from F, Cl, and I, n and r are preferably 0 or more.

A is an organic group having 6 to 30 carbon atoms. A may be a monocyclic organic group or a polycyclic organic group, or optionally has a substituent. A is preferably an aromatic ring optionally having a substituent. The number of carbon atoms of A is preferably 6 to 14, and more preferably 6 to 10. A is preferably a group represented by any of the following formula (A-1) to formula (A-4), more preferably a group represented by the following formula (A-1) to formula (A-2), and further preferably a group represented by the following formula (A-1).

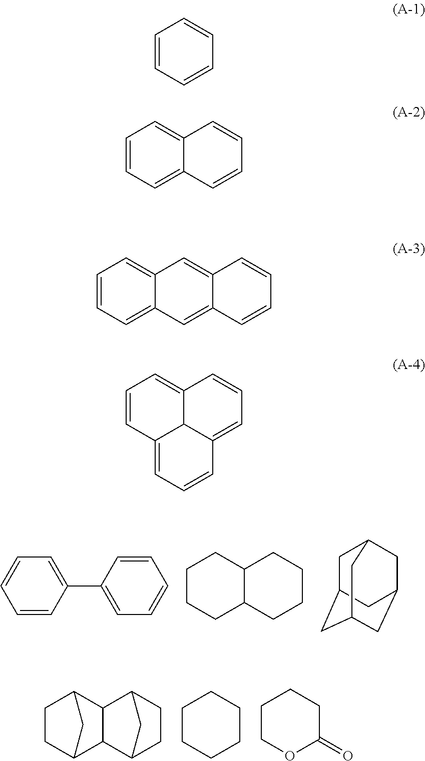

A may be an alicyclic structure optionally having a substituent. Here, the “alicyclic structure” refers to a saturated or unsaturated carbocycle having no aromatic properties. Examples of the alicyclic structure include a saturated or unsaturated carbocycle having 6 to 30 carbon atoms, and a saturated or unsaturated carbocycle having 6 to 20 carbon atoms is preferable. Examples of the alicyclic structure include a group having cyclohexyl, cycloheptyl, cyclooctyl, cyclononyl, cyclodecyl, cycloicosyl, cyclopropenyl, cyclobutenyl, cyclopentenyl, cyclohexenyl, cycloheptenyl, cyclooctenyl, cyclopentadienyl, cyclooctadienyl, adamantyl, bicycloundecyl, decahydronaphthyl, norbornyl, norbornadienyl, cubane, basketane, or housane.

A may also be a heterocyclic structure optionally having a substituent. Examples of the heterocyclic structure include, but are not particularly limited to, a nitrogen-containing cyclic structure such as pyridine, piperidine, piperidone, benzodiazole, and benzotriazole; a cyclic ether such as triazine, a cyclic urethane structure, cyclic urea, cyclic amide, cyclic imide, furan, pyran, and dioxolane; and an alicyclic group having a lactone structure such as caprolactone, butyrolactone, nonalactone, decalactone, undecalactone, bicycloundecalactone, and phthalide.

p is an integer of 1 or more, preferably an integer of 1 or more and 3 or less, more preferably an integer of 1 or more and 2 or less, and further preferably 1.

Each Z is independently an alkoxy group, an ester group, an acetal group, a carboxyalkoxy group, or a carbonate ester group. These groups optionally have a substituent, and examples of the substituent include a hydrocarbon group having 1 to 60 carbon atoms and optionally further having a substituent. r is an integer of 0 or more, preferably an integer of 0 or more and 2 or less, more preferably an integer of 0 or more and 1 or less, and further preferably 1.

Examples of Z include at least one group selected from the group consisting of an alkoxy group [*³—O—R²], an ester group [*³—O—(C═O)—R² or *³—(C═O)—O—R²], an acetal group [*³—O—(C(R²¹)₂)—O—R² (wherein each R²¹ is independently H or a hydrocarbon group having 1 to 10 carbon atoms)], a carboxyalkoxy group [*³—O—R²²—(C═O)—O—R² (wherein R²² is a divalent hydrocarbon group having 1 to 10 carbon atoms)], and a carbonate ester group [*³—O—(C═O)—O—R²]. The ester group is preferably a tertiary ester group, from the viewpoint of achieving high sensitivity. In the formula, *³ is a site for binding with A. Among them, Z is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of achieving high exposure sensibility of a resist composition. In the production method of the compound A by the present invention, from the viewpoint of improving the stability of an intermediate compound in an acidic to weakly acidic process and from the viewpoint of producing a polymer having a stable quality by radical polymerization when the compound A is used as a resist material, Z is preferably an ester group, a carboxyalkoxy group, or a carbonate ester group.

As described above, n is an integer of 0 or more, r is an integer of 0 or more, but at least one of n or r may be an integer of 1 or more. That is, n+r may be an integer of 1 or more.

Among the above compounds A, the compound represented by the following formula (0b) or formula (5b) is preferable.

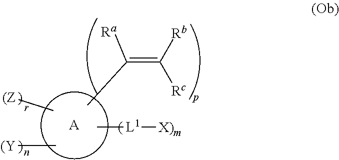

In the formula (0b),
X, L¹, Y, A, Z, p, m, n, r, R^{a}, R^{b}, and R^{c} are as defined in the formula (1).

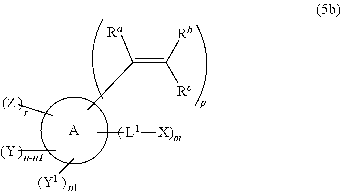

In the formula (5b),
X, L¹, Y, R^{a}, R^{b}, R^{c}, Z, A, m, n, p, and r are as defined in the formula (1);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally have a substituent; and
n1 is an integer of 1 or more and n or less.

Examples of the compound A represented by the formula (1) in the present embodiment include, but are not particularly limited to, compounds having the structures given below.

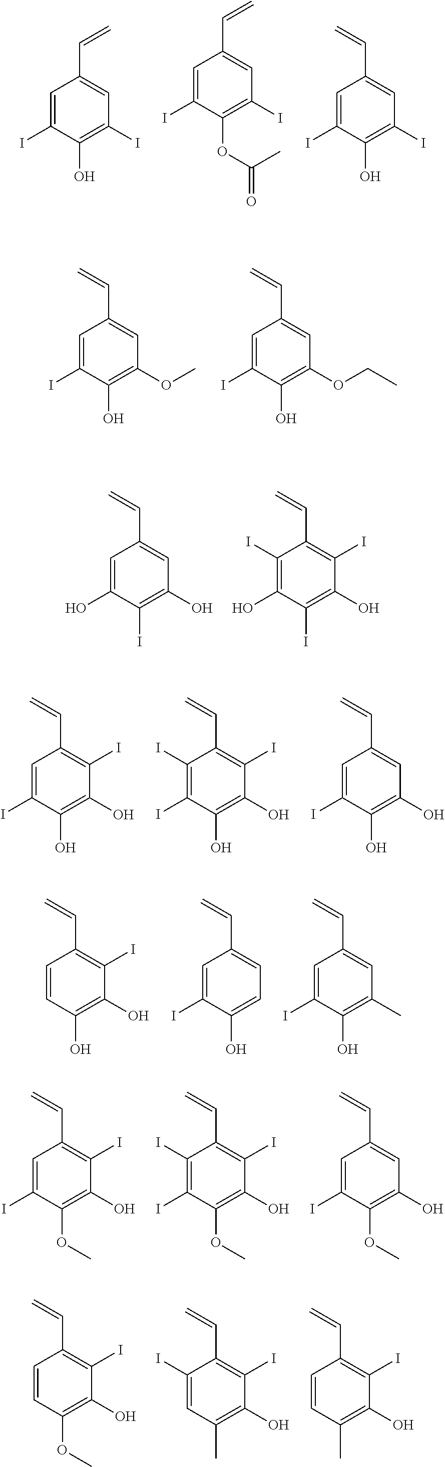

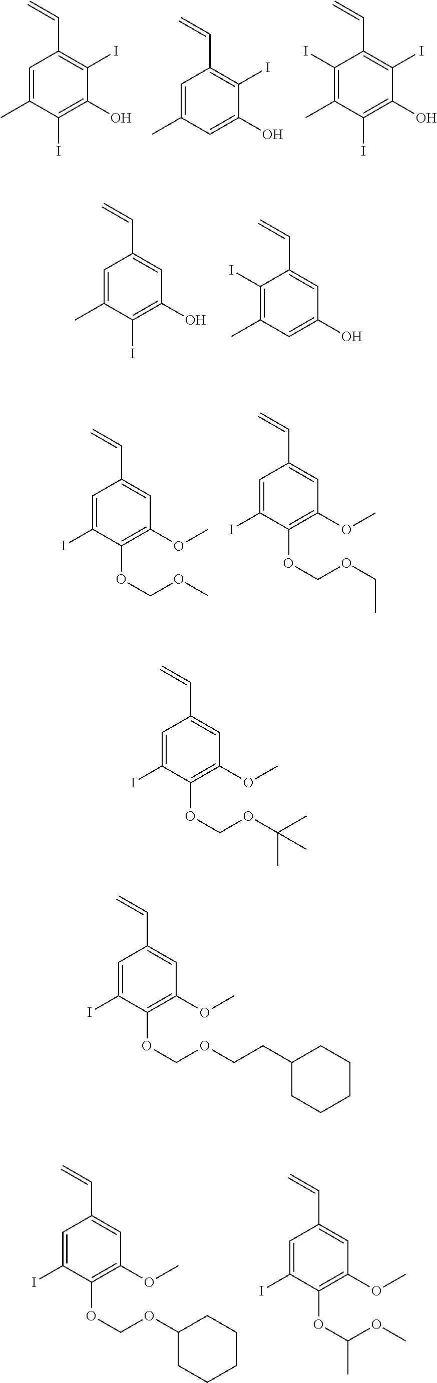

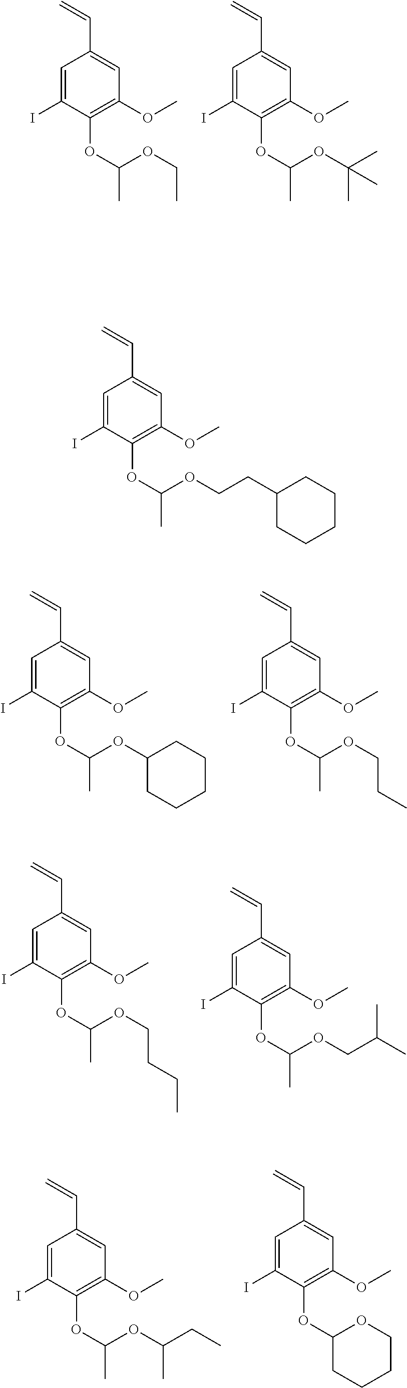

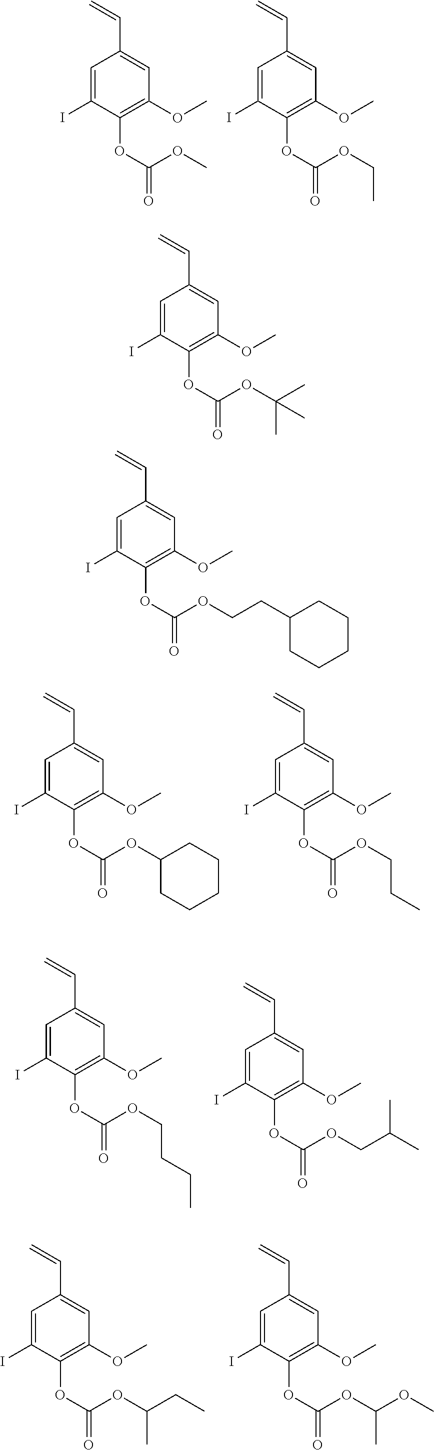

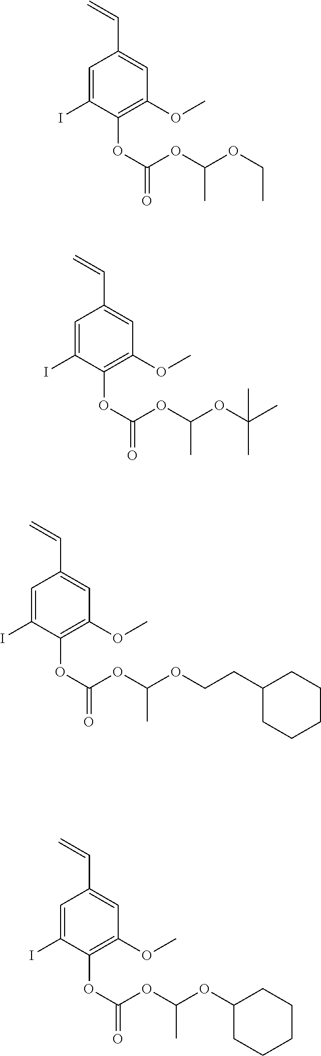 [SMILES: C=Cc1cc(I)c(OC(=O)OC(C)OCC)c(OC)c1 ; C=Cc1cc(I)c(OC(=O)OC(C)OC(C)(C)C)c(OC)c1 ; C=Cc1cc(I)c(OC(=O)OC(C)OCCC2CCCCC2)c(OC)c1 ; C=Cc1cc(I)c(OC(=O)OC(C)OC2CCCCC2)c(OC)c1]

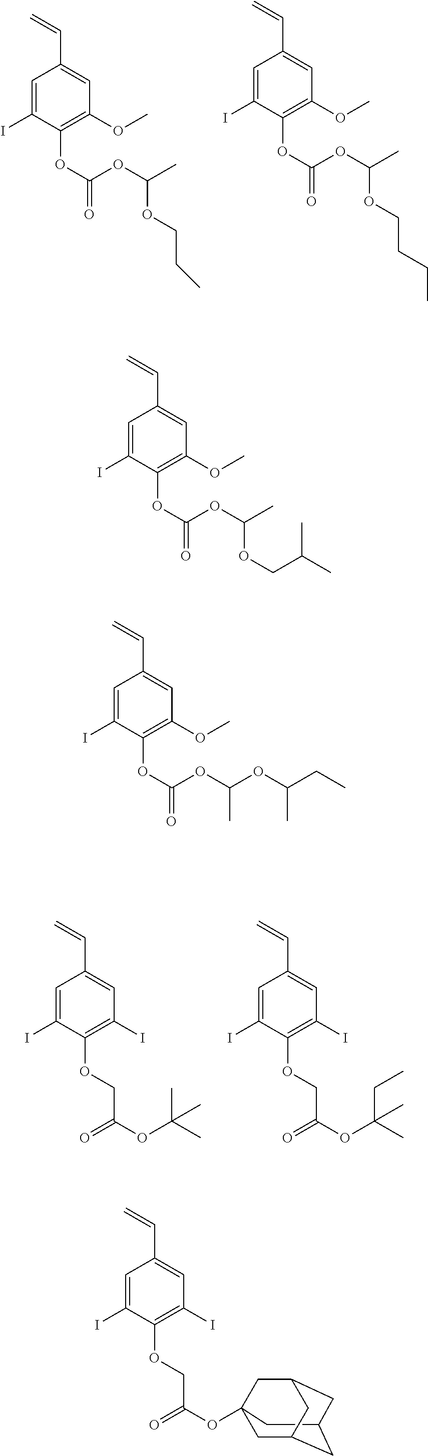

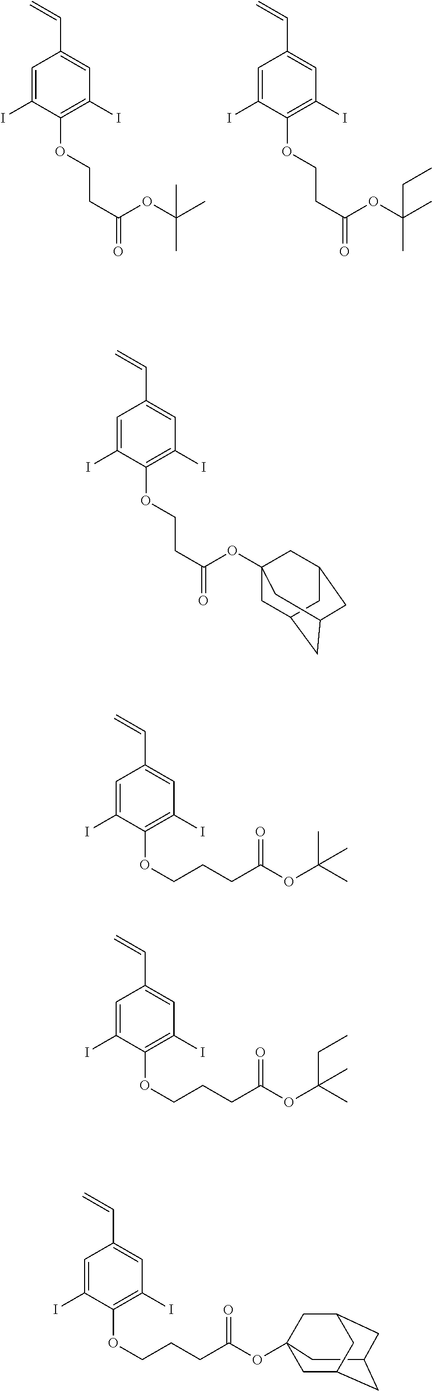 [SMILES: C=Cc1cc(I)c(OCCC(=O)OC(C)(C)C)c(I)c1 ; C=Cc1cc(I)c(OCCC(=O)OC(C)(C)CC)c(I)c1 ; C=Cc1cc(I)c(OCCC(=O)OC23CC4CC(CC(C4)C2)C3)c(I)c1 ; C=Cc1cc(I)c(OCCCC(=O)OC(C)(C)C)c(I)c1 ; C=Cc1cc(I)c(OCCCC(=O)OC(C)(C)CC)c(I)c1 ; C=Cc1cc(I)c(OCCCC(=O)OC23CC4CC(CC(C4)C2)C3)c(I)c1]

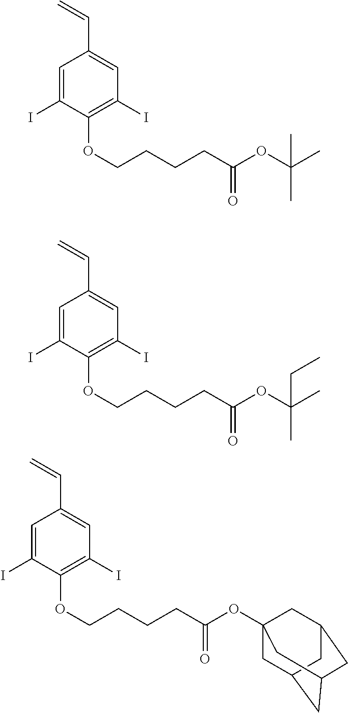 [SMILES: C=Cc1cc(I)c(OCCCCC(=O)OC(C)(C)C)c(I)c1 ; C=Cc1cc(I)c(OCCCCC(=O)OC(C)(C)CC)c(I)c1 ; C=Cc1cc(I)c(OCCCCC(=O)OC23CC4CC(CC(C4)C2)C3)c(I)c1]

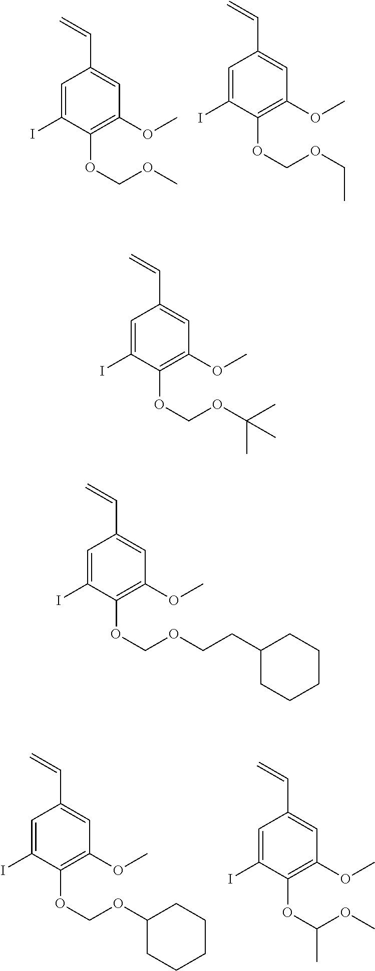

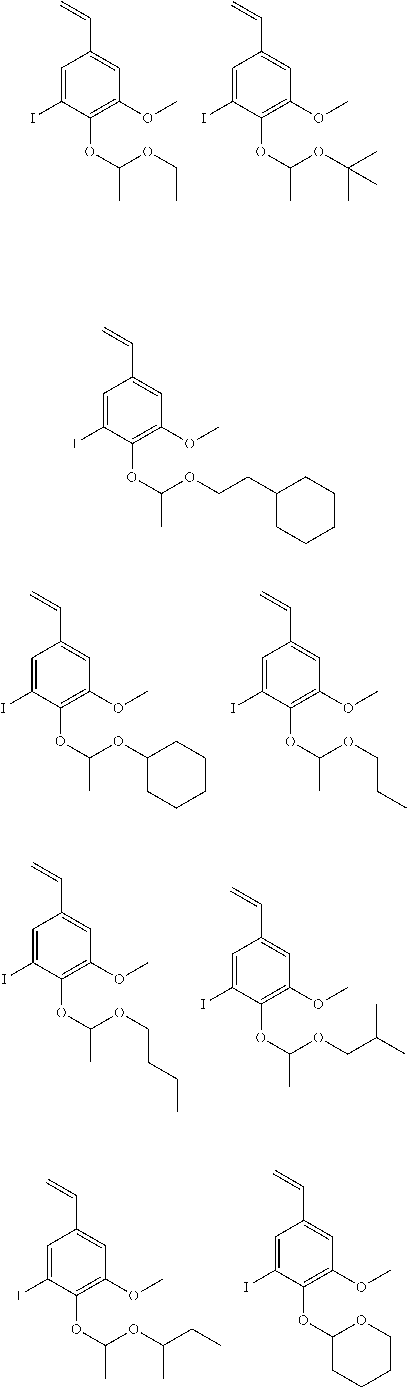

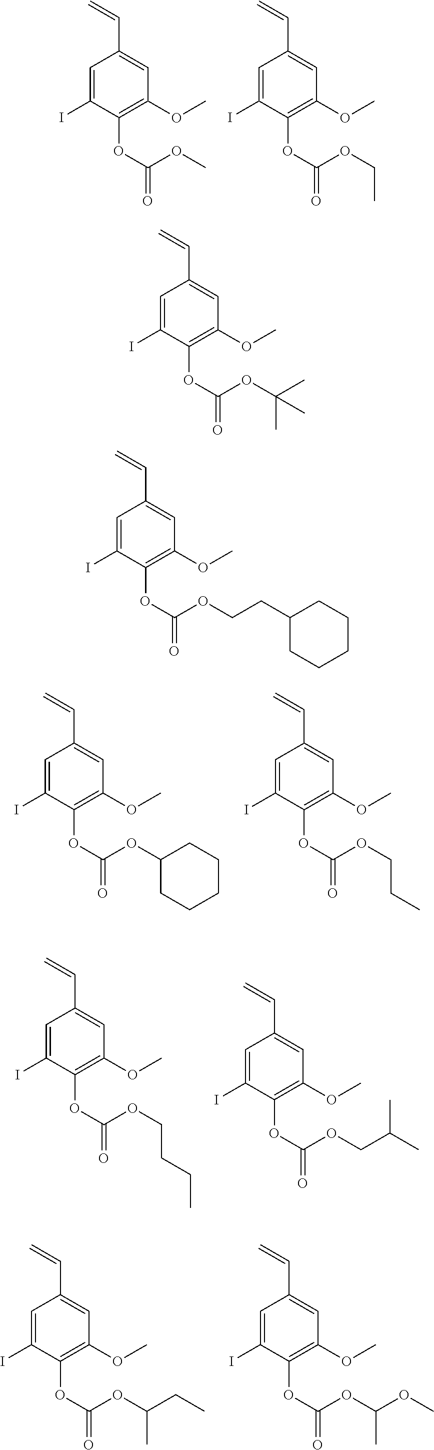

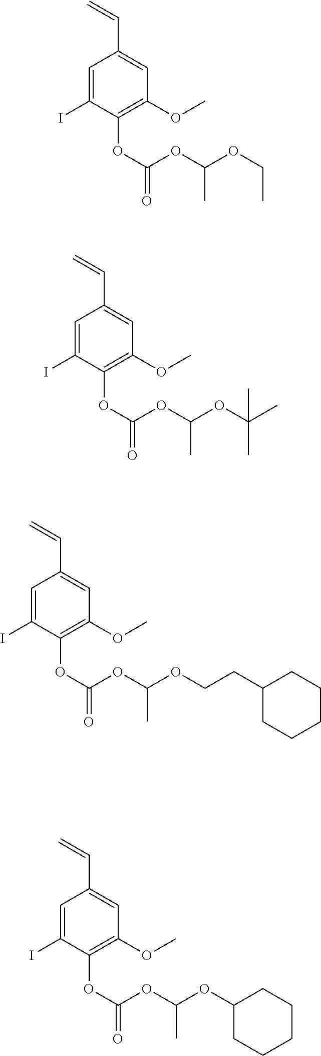 [SMILES: C=Cc1cc(I)c(OC(=O)OC(C)OCC)c(OC)c1 ; C=Cc1cc(I)c(OC(=O)OC(C)OC(C)(C)C)c(OC)c1 ; C=Cc1cc(I)c(OC(=O)OC(C)OCCC2CCCCC2)c(OC)c1 ; C=Cc1cc(I)c(OC(=O)OC(C)OC2CCCCC2)c(OC)c1]

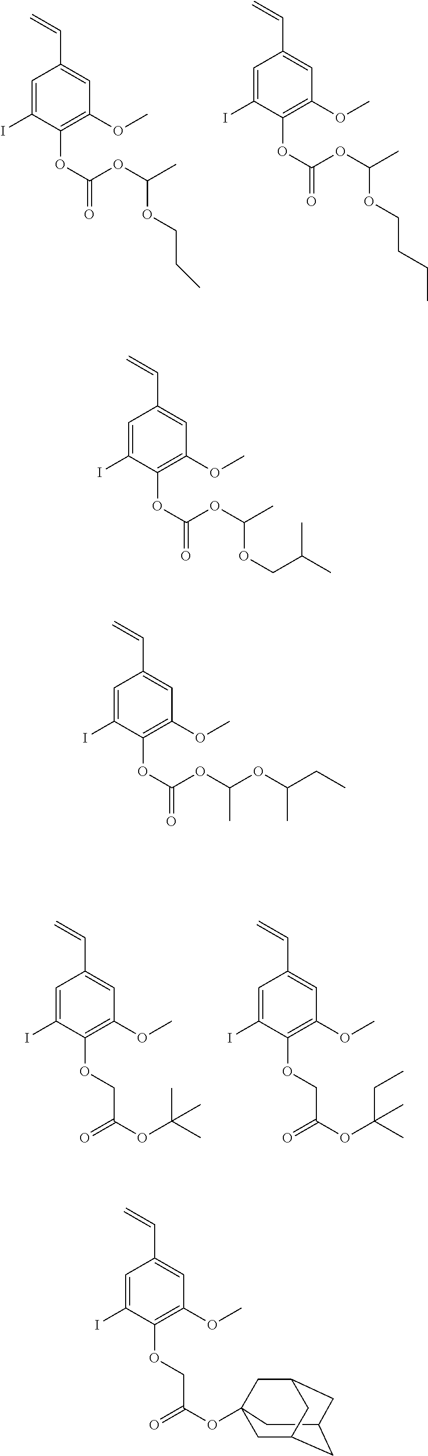

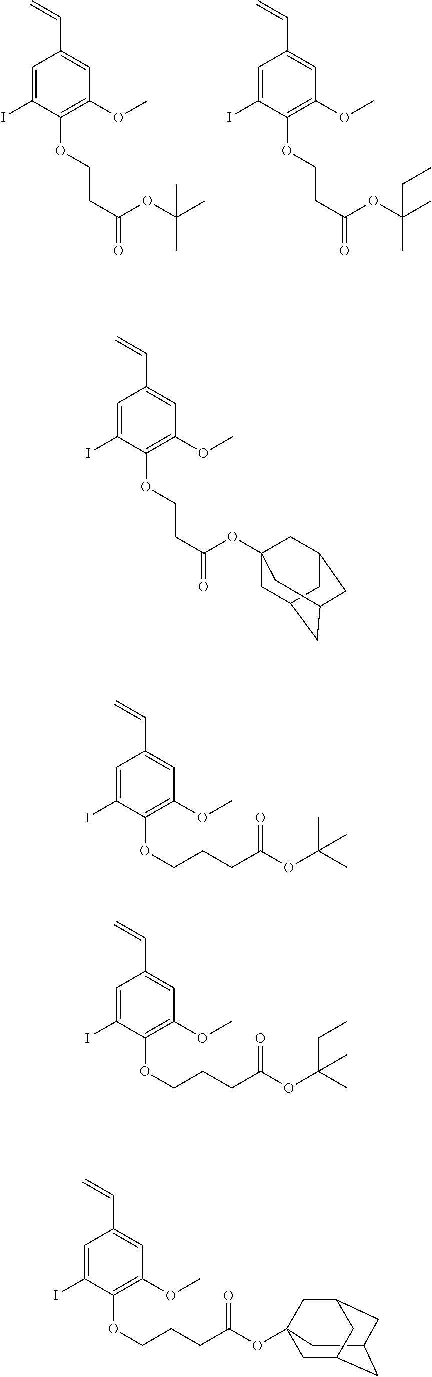 [SMILES: C=Cc1cc(I)c(OCCC(=O)OC(C)(C)C)c(OC)c1 ; C=Cc1cc(I)c(OCCC(=O)OC(C)(C)CC)c(OC)c1 ; C=Cc1cc(I)c(OCCC(=O)OC23CC4CC(CC(C4)C2)C3)c(OC)c1 ; C=Cc1cc(I)c(OCCCC(=O)OC(C)(C)C)c(OC)c1 ; C=Cc1cc(I)c(OCCCC(=O)OC(C)(C)CC)c(OC)c1 ; C=Cc1cc(I)c(OCCCC(=O)OC23CC4CC(CC(C4)C2)C3)c(OC)c1]

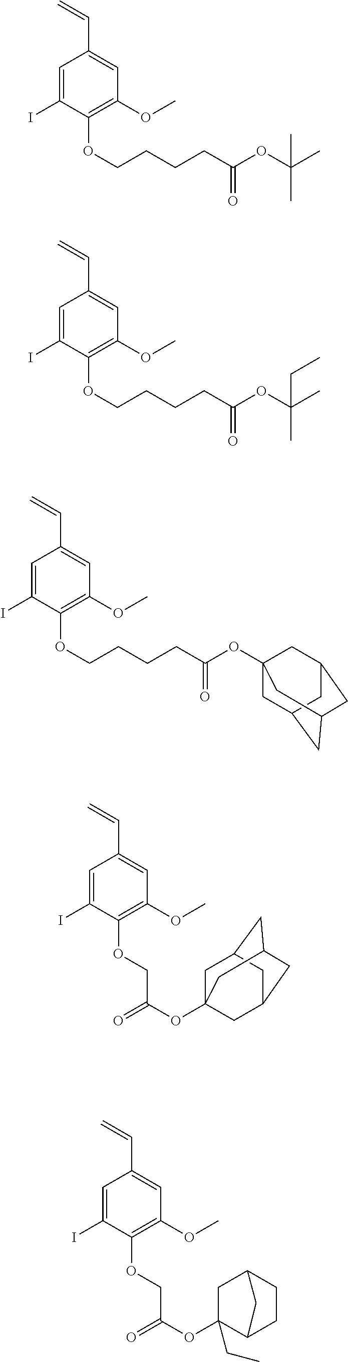 [SMILES: C=Cc1cc(I)c(OCCCCC(=O)OC(C)(C)C)c(OC)c1 ; C=Cc1cc(I)c(OCCCCC(=O)OC(C)(C)CC)c(OC)c1 ; C=Cc1cc(I)c(OCCCCC(=O)OC23CC4CC(CC(C4)C2)C3)c(OC)c1 ; C=Cc1cc(I)c(OCC(=O)OC23CC4CC(CC(C4)C2)C3)c(OC)c1 ; C=Cc1cc(I)c(OCC(=O)OC2(CC)CC3CCC2C3)c(OC)c1]

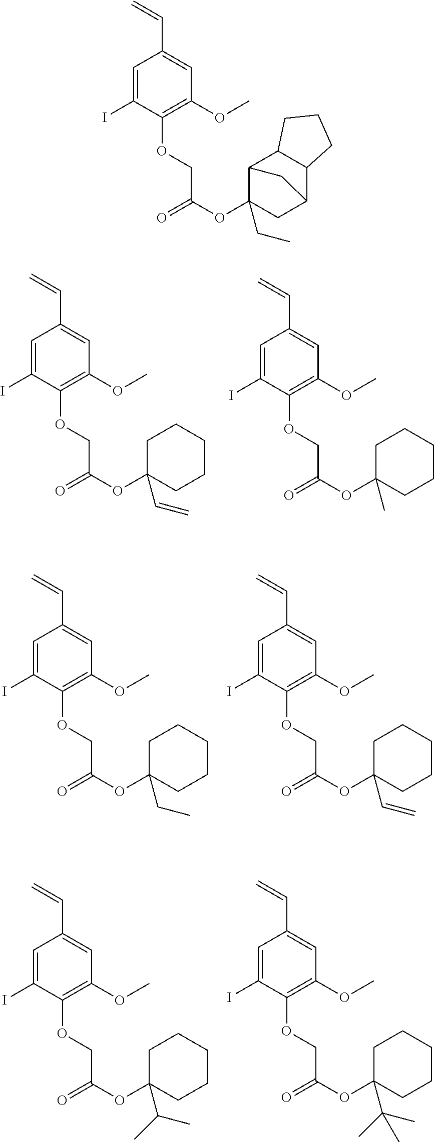

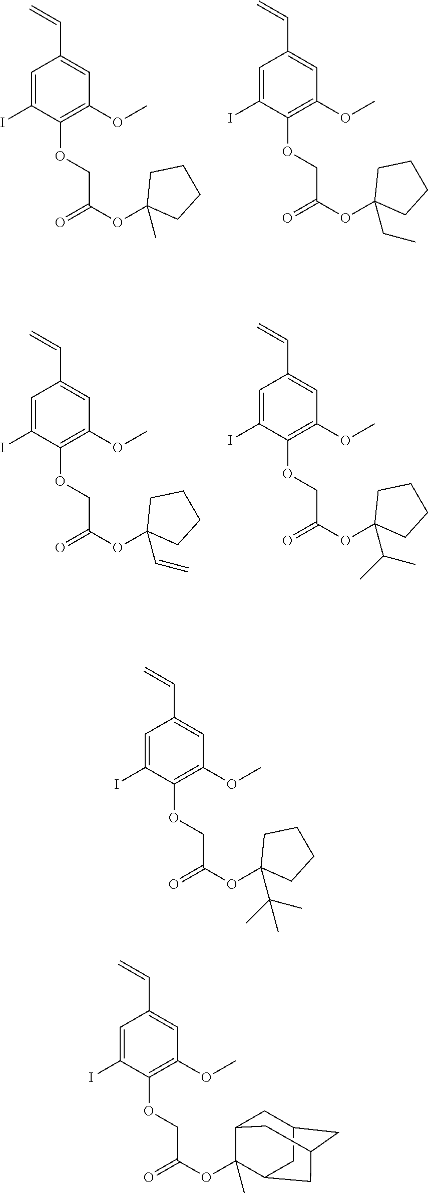 [SMILES: C=Cc1cc(I)c(OCC(=O)OC2(C)CCCC2)c(OC)c1 ; C=Cc1cc(I)c(OCC(=O)OC2(CC)CCCC2)c(OC)c1 ; C=Cc1cc(I)c(OCC(=O)OC2(C=C)CCCC2)c(OC)c1 ; C=Cc1cc(I)c(OCC(=O)OC2(C(C)C)CCCC2)c(OC)c1 ; C=Cc1cc(I)c(OCC(=O)OC2(C(C)(C)C)CCCC2)c(OC)c1 ; C=Cc1cc(I)c(OCC(=O)OC2(C)C3CC4CC(C3)CC2C4)c(OC)c1]

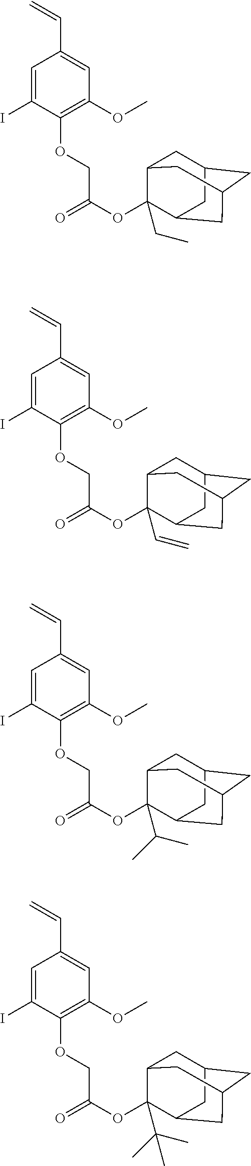 [SMILES: C=Cc1cc(I)c(OCC(=O)OC2(CC)C3CC4CC(C3)CC2C4)c(OC)c1 ; C=Cc1cc(I)c(OCC(=O)OC2(C=C)C3CC4CC(C3)CC2C4)c(OC)c1 ; C=Cc1cc(I)c(OCC(=O)OC2(C(C)C)C3CC4CC(C3)CC2C4)c(OC)c1 ; C=Cc1cc(I)c(OCC(=O)OC2(C(C)(C)C)C3CC4CC(C3)CC2C4)c(OC)c1]

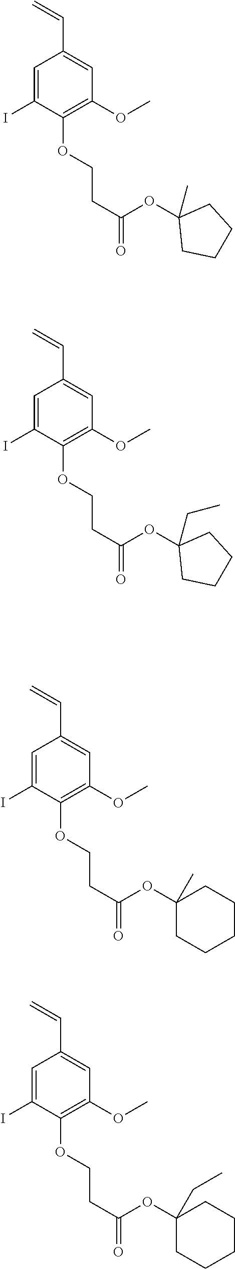 [SMILES: C=Cc1cc(I)c(OCCC(=O)OC2(C)CCCC2)c(OC)c1 ; C=Cc1cc(I)c(OCCC(=O)OC2(CC)CCCC2)c(OC)c1 ; C=Cc1cc(I)c(OCCC(=O)OC2(C)CCCCC2)c(OC)c1 ; C=Cc1cc(I)c(OCCC(=O)OC2(CC)CCCCC2)c(OC)c1]

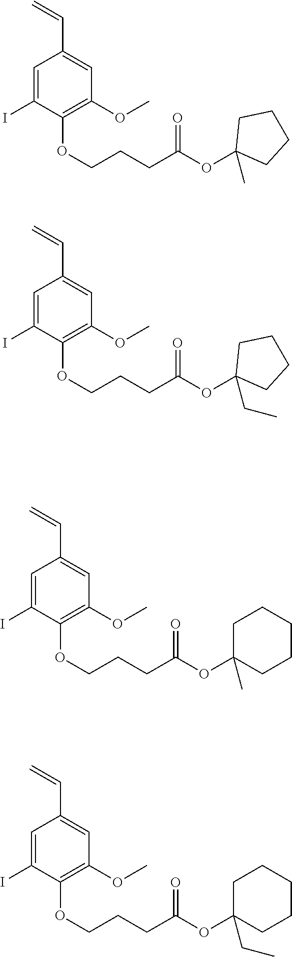 [SMILES: C=Cc1cc(I)c(OCCCC(=O)OC2(C)CCCC2)c(OC)c1 ; C=Cc1cc(I)c(OCCCC(=O)OC2(CC)CCCC2)c(OC)c1 ; C=Cc1cc(I)c(OCCCC(=O)OC2(C)CCCCC2)c(OC)c1 ; C=Cc1cc(I)c(OCCCC(=O)OC2(CC)CCCCC2)c(OC)c1]

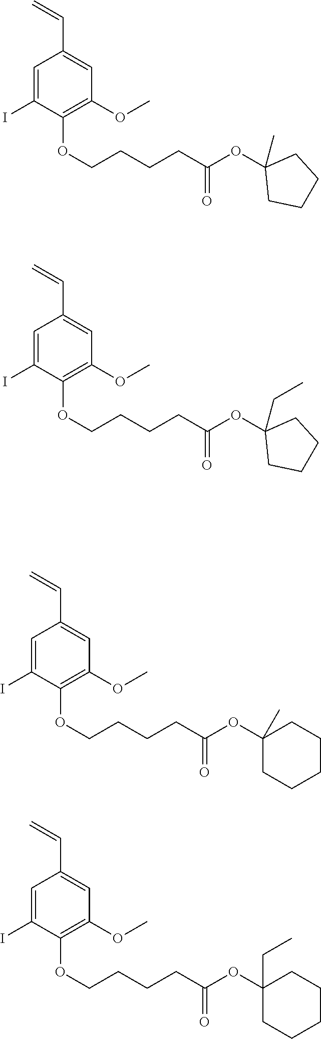 [SMILES: C=Cc1cc(I)c(OCCCCC(=O)OC2(C)CCCC2)c(OC)c1 ; C=Cc1cc(I)c(OCCCCC(=O)OC2(CC)CCCC2)c(OC)c1 ; C=Cc1cc(I)c(OCCCCC(=O)OC2(C)CCCCC2)c(OC)c1 ; C=Cc1cc(I)c(OCCCCC(=O)OC2(CC)CCCCC2)c(OC)c1]

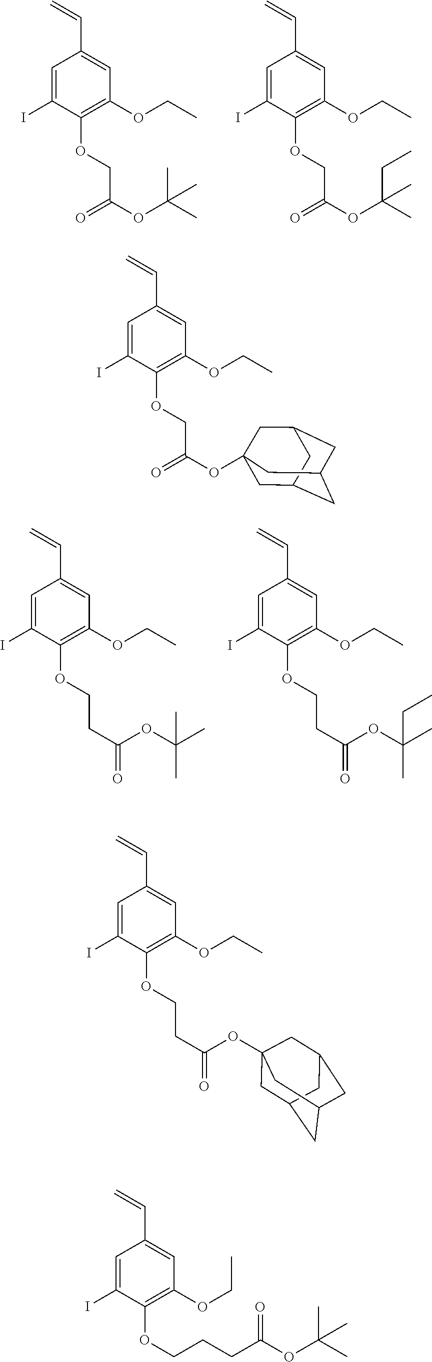

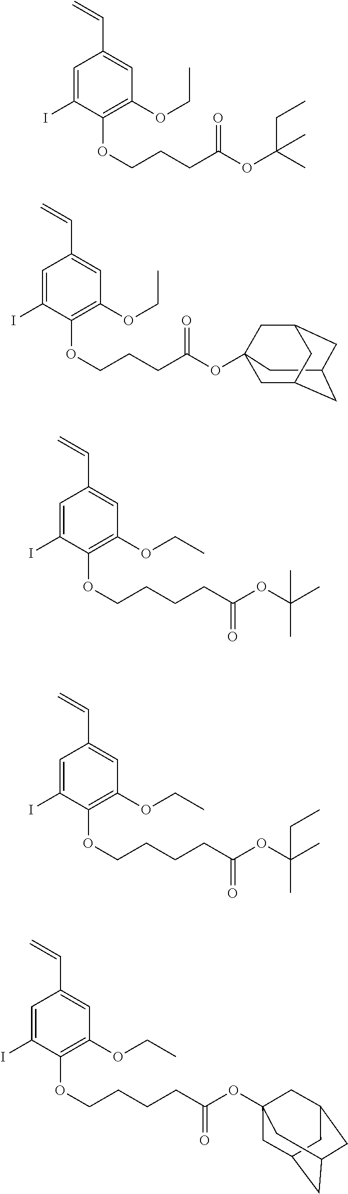 [SMILES: C=Cc1cc(I)c(OCCCC(=O)OC(C)(C)CC)c(OCC)c1 ; C=Cc1cc(I)c(OCCCC(=O)OC23CC4CC(CC(C4)C2)C3)c(OCC)c1 ; C=Cc1cc(I)c(OCCCCC(=O)OC(C)(C)C)c(OCC)c1 ; C=Cc1cc(I)c(OCCCCC(=O)OC(C)(C)CC)c(OCC)c1 ; C=Cc1cc(I)c(OCCCCC(=O)OC23CC4CC(CC(C4)C2)C3)c(OCC)c1]

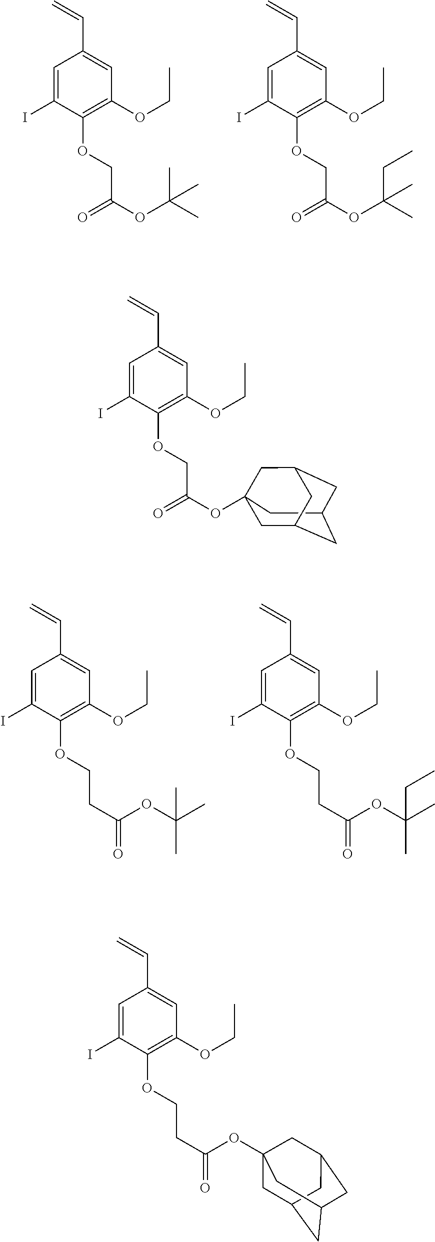 [SMILES: C=Cc1cc(I)c(OCC(=O)OC(C)(C)C)c(OCC)c1 ; C=Cc1cc(I)c(OCC(=O)OC(C)(C)CC)c(OCC)c1 ; C=Cc1cc(I)c(OCC(=O)OC23CC4CC(CC(C4)C2)C3)c(OCC)c1 ; C=Cc1cc(I)c(OCCC(=O)OC(C)(C)C)c(OCC)c1 ; C=Cc1cc(I)c(OCCC(=O)OC(C)(C)CC)c(OCC)c1 ; C=Cc1cc(I)c(OCCC(=O)OC23CC4CC(CC(C4)C2)C3)c(OCC)c1]

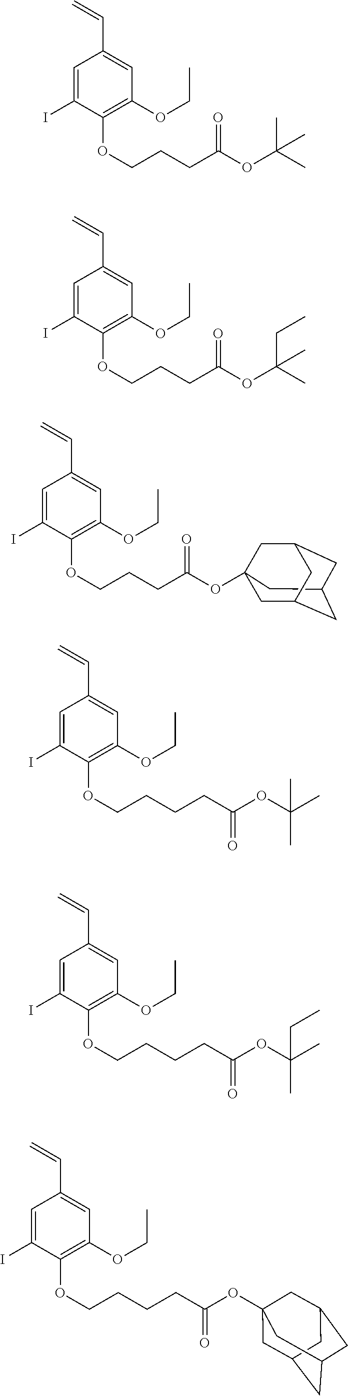 [SMILES: C=Cc1cc(I)c(OCCCC(=O)OC(C)(C)C)c(OCC)c1 ; C=Cc1cc(I)c(OCCCC(=O)OC(C)(C)CC)c(OCC)c1 ; C=Cc1cc(I)c(OCCCC(=O)OC23CC4CC(CC(C4)C2)C3)c(OCC)c1 ; C=Cc1cc(I)c(OCCCCC(=O)OC(C)(C)C)c(OCC)c1 ; C=Cc1cc(I)c(OCCCCC(=O)OC(C)(C)CC)c(OCC)c1 ; C=Cc1cc(I)c(OCCCCC(=O)OC23CC4CC(CC(C4)C2)C3)c(OCC)c1]

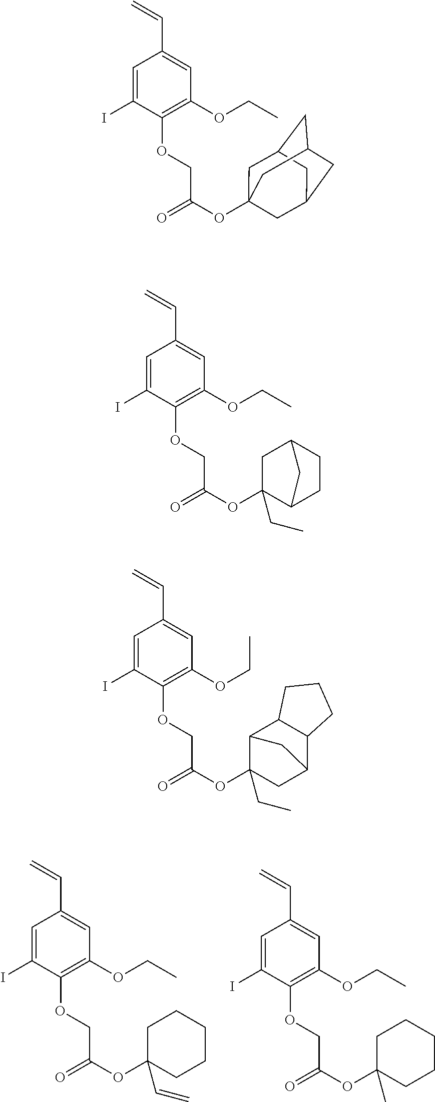 [SMILES: C=Cc1cc(I)c(OCC(=O)OC23CC4CC(CC(C4)C2)C3)c(OCC)c1 ; C=Cc1cc(I)c(OCC(=O)OC2(CC)CC3CCC2C3)c(OCC)c1 ; C=Cc1cc(I)c(OCC(=O)OC2(CC)CC3CC2C2CCCC32)c(OCC)c1 ; C=Cc1cc(I)c(OCC(=O)OC2(C=C)CCCCC2)c(OCC)c1 ; C=Cc1cc(I)c(OCC(=O)OC2(C)CCCCC2)c(OCC)c1]

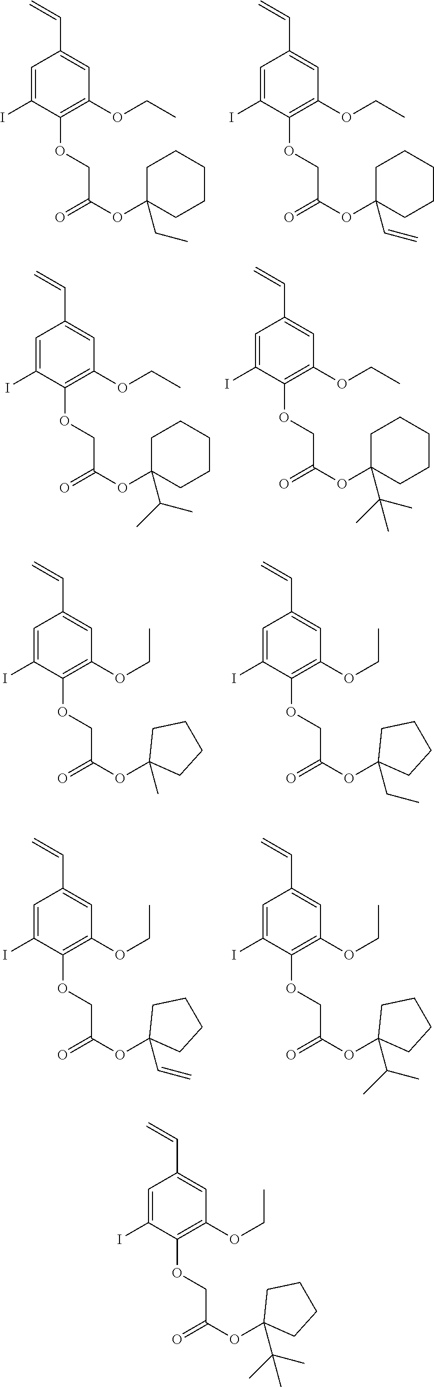

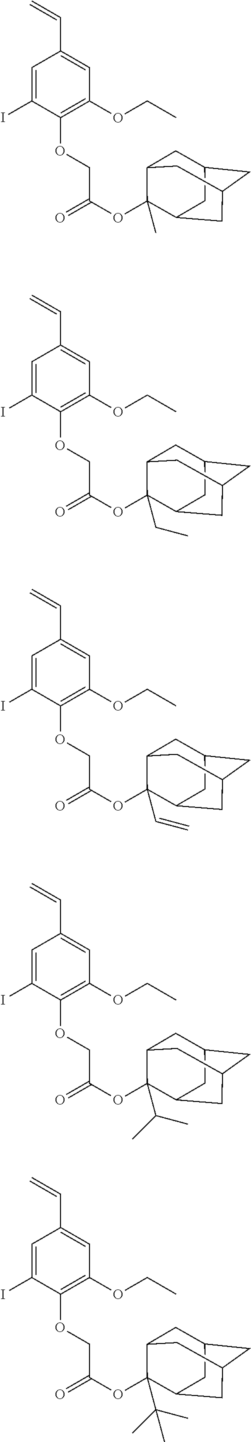 [SMILES: C=Cc1cc(I)c(OCC(=O)OC2(C)C3CC4CC(C3)CC2C4)c(OCC)c1 ; C=Cc1cc(I)c(OCC(=O)OC2(CC)C3CC4CC(C3)CC2C4)c(OCC)c1 ; C=Cc1cc(I)c(OCC(=O)OC2(C=C)C3CC4CC(C3)CC2C4)c(OCC)c1 ; C=Cc1cc(I)c(OCC(=O)OC2(C(C)C)C3CC4CC(C3)CC2C4)c(OCC)c1 ; C=Cc1cc(I)c(OCC(=O)OC2(C(C)(C)C)C3CC4CC(C3)CC2C4)c(OCC)c1]

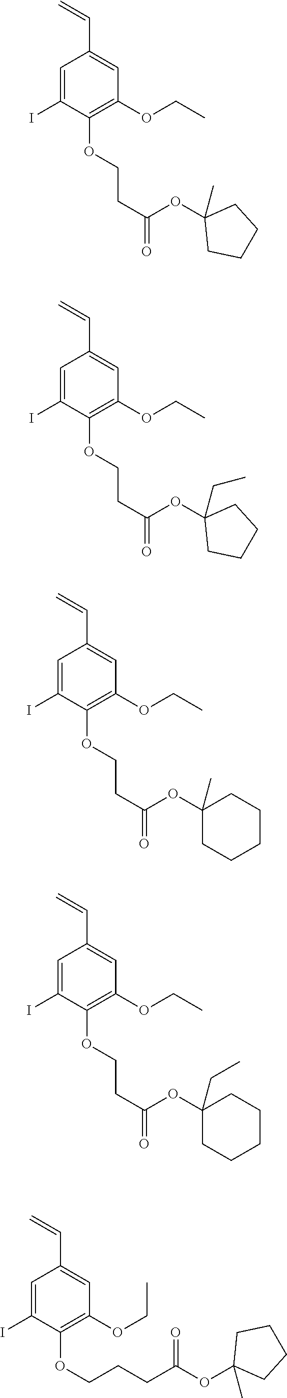 [SMILES: C=Cc1cc(I)c(OCCC(=O)OC2(C)CCCC2)c(OCC)c1 ; C=Cc1cc(I)c(OCCC(=O)OC2(CC)CCCC2)c(OCC)c1 ; C=Cc1cc(I)c(OCCC(=O)OC2(C)CCCCC2)c(OCC)c1 ; C=Cc1cc(I)c(OCCC(=O)OC2(CC)CCCCC2)c(OCC)c1 ; C=Cc1cc(I)c(OCCCC(=O)OC2(C)CCCC2)c(OCC)c1]

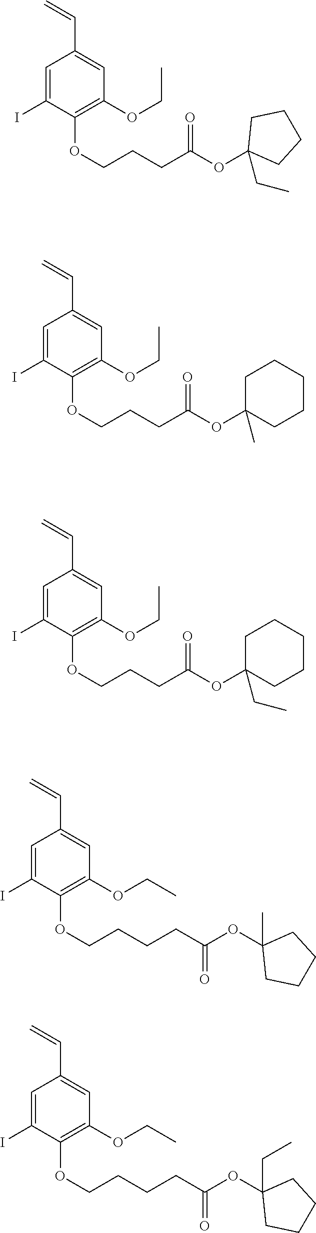 [SMILES: C=Cc1cc(I)c(OCCCC(=O)OC2(CC)CCCC2)c(OCC)c1 ; C=Cc1cc(I)c(OCCCC(=O)OC2(C)CCCCC2)c(OCC)c1 ; C=Cc1cc(I)c(OCCCC(=O)OC2(CC)CCCCC2)c(OCC)c1 ; C=Cc1cc(I)c(OCCCCC(=O)OC2(C)CCCC2)c(OCC)c1 ; C=Cc1cc(I)c(OCCCCC(=O)OC2(CC)CCCC2)c(OCC)c1]

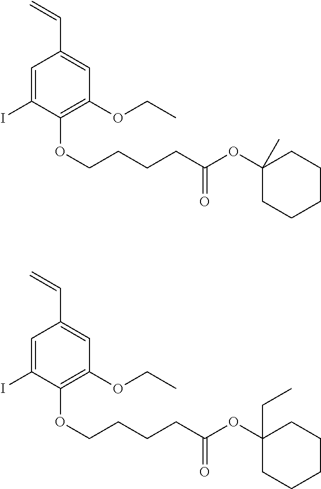 [SMILES: C=Cc1cc(I)c(OCCCCC(=O)OC2(C)CCCCC2)c(OCC)c1 ; C=Cc1cc(I)c(OCCCCC(=O)OC2(CC)CCCCC2)c(OCC)c1]

### (Method for Producing Compound A)

In the present embodiment, the production method of the compound A comprises at least one of step HX, step ST, and step PR. Here, step HX is a step of introducing a halogen or a group containing a halogen, and specifically a step of introducing a halogen or a group containing a halogen into a target compound. Step ST is a step of introducing an unsaturated double bond, and specifically a step of introducing an unsaturated double bond into a target compound. Step PR is a step of introducing a group represented by the following formula (Y-0), and specifically a step of introducing an alkoxy group, an ester group, an ether group, a thioether group, an acetal group, a thioacetal group, a carboxyalkoxy group, a carbonate ester group, a sulfonyl group, and a silyl group, or a hydrolyzable group into a target compound.

In the production method of the present embodiment, the order of step HX, step ST, and step PR is not particularly limited, as long as at least one of step HX, step ST, and step PR is included, and each of step HX, step ST, and step PR may be repeated a plurality of times. In addition, a step other than step HX, step ST, and step PR may be carried out between respective steps of step HX, step ST, and step PR. Note that, when the production method of the present embodiment comprises step ST and step PR, these steps may be step STPR of carrying out one step of step ST and step PR and then continuously carrying out the other step without carrying out isolation and purification operation. Step STPR will be described below.

### <Step HX>

In the present embodiment, step HX is a step of introducing a halogen or a group containing a halogen into a target compound. Examples of step HX include, but are not particularly limited to, a method for introducing a halogen from an amino group by the Sandmeyer reaction or the like, a method for reacting iodine chloride in an organic solvent (e.g., Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012). The halogen or group containing a halogen to be introduced is not particularly limited, and is preferably F, I, or a group containing F or I, and more preferably I or a group containing I, from the viewpoint of the performance when the compound A obtained by the production method of the present embodiment is applied to a resist resin, in particular, the effect of improving the sensibility to EUV.

Examples of the halogenating agent include, but are not particularly limited to, an iodinating agent such as iodine chloride, iodine, and N-iodosuccinimide; a fluorinating agent such as potassium fluoride and tetramethylammonium fluoride; a chlorinating agent such as thionyl chloride and dichloromethylmethyl ether; and a brominating agent such as a bromine molecule, carbon tetrabromide, and N-bromosuccinimide. Among them, an iodinating agent is preferable, iodine or iodine chloride is more preferable, and iodine is particularly preferable.

The ratio of the halogenating agent to the substrate in step HX is preferably 1.2 mol times or more, more preferably 1.5 mol times or more, and further preferably 2.0 mol times or more.

In step HX, when I or a group containing I is introduced into a target compound, the iodine introduction reaction can be proceed by reacting at least an iodizing agent with a substrate. More specifically, a compound into which I or a group containing a group containing I is introduced can be obtained by known iodine introduction reaction conditions using a method described in Non-Patent Literature such as Adv. Synth. Catal. 2007, 349, 1159-1172, Organic Letters; Vol. 6; (2004); p. 2785-2788 or Patent Literature such as U.S. Pat. Nos. 5,300,506, 5,434,154, US2009/281114, EP1439164, and International Publication No. WO 2006/101318, without particular limitation.

Examples of the iodinating agent that can be used include, but are not particularly limited to, iodine chloride, iodine, N-iodosuccinimide, iodine compounds, monochloride iodine, N-iodosuccinimide, benzyltrimethylammonium dichloroiodate, tetraethylammonium iodide, tetranormalbutylammonium iodide, lithium iodide, sodium iodide, potassium iodide, 1-chloro-2-iodoethane, iodine silver fluoride, tert-butyl hypoiodite, 1,3-diiodo-5,5-dimethylhydantoin, iodine-morpholine complexes, trifluoroacetyl hypoiodite, iodine-iodic acid, iodine-periodic acid, iodine-hydrogen peroxide, 1-iodoheptafluoropropane, triphenylphosphate-methyl iodide, iodine-thallium (I) acetate, 1-chloro-2-iodoethane, and iodine-copper (II) acetate.

In step HX, when I or a group containing a group containing I is introduced, one or more additives may be added in the iodine introduction reaction to promote the reaction or to suppress by-products. Examples of the additive include, but are not particularly limited to, an acid such as hydrochloric acid, sulfuric acid, nitric acid, phosphoric acid, acetic acid, p-toluenesulfonic acid, ferric chloride, aluminum chloride, copper chloride, antimony pentachloride, silver sulfate, silver nitrate, and silver trifluoroacetate; a base such as sodium hydroxide, potassium hydrate, lithium hydroxide, sodium carbonate, potassium carbonate, calcium carbonate, sodium bicarbonate, and potassium bicarbonate; an oxidizing agent such as ammonium cerium (IV) nitrate and sodium peroxodisulfate; an inorganic compound such as sodium chloride, potassium chloride, mercury (II) oxide, and cerium oxide; an organic compound such as acetic anhydride; and zeolite.

In step HX, from the viewpoint of improving reaction efficiency and purity, iodine is preferably introduced using at least an iodine source and an oxidizing agent. Examples of the iodine source include, but are not particularly limited to, the above iodinating agents. Examples of the oxidizing agent include, but are not particularly limited to, periodic acid, hydrogen peroxide, and a predetermined additive (such as hydrochloric acid, sulfuric acid, nitric acid, and p-toluenesulfonic acid).

In step HX, the reaction of introducing a halogen or a group containing a halogen may be conducted in the absence of a solvent or may be conducted using a solvent. Examples of the solvent include, but are not particularly limited to, a halogenated solvent such as dichloromethane, dichloroethane, chloroform, and carbon tetrachloride; an alkyl solvent such as hexane, cyclohexane, heptane, pentane, and octane; an aromatic hydrocarbon solvent such as benzene and toluene; an alcohol solvent such as methanol, ethanol, 1-propanol, 2-propanol, 1-butanol, and 2-butanol; an ether solvent such as diethyl ether, diisopropylether, tetrahydrofuran, cyclopentyl methyl ether, 1,4-dioxane, and diglyme; acetic acid, dimethylformamide, dimethylsulfoxide, and water.

In step HX, when I or a group containing a group containing I is introduced, the iodine introduction reaction can be proceed by reacting at least an iodizing agent with a substrate (target compound). For example, the target compound can be obtained under known iodine introduction reaction conditions by the Sandmeyer reaction or the like using a method described in Chemistry—A European Journal, 24(55), 14622-14626; 2018, Synthesis (2007)(1), 81-84 or the like.

In step HX, the reaction temperature of the reaction for introducing a halogen or a group containing a halogen is not particularly limited. The reaction temperature may be any temperature from the freezing point to the boiling point of the solvent used in the reaction, and is preferably 0° C. or more and 150° C. or less.

In step HX of the present embodiment, the substrate (target compound) into which a halogen or a group containing a halogen is introduced is not particularly limited, and the compounds having a reaction point of the halogen introduction reaction mentioned above can be used.

In the present embodiment, step HX may be a step of introducing a halogen or a group containing a halogen into a compound represented by the following formula (3a), the following formula (0a), or the following formula (4a).

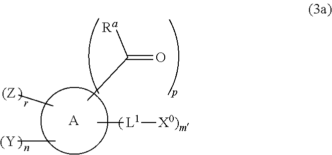

(In the formula (3a),
L¹, Y, R^{a}, Z, A, n, p, and r are as defined in the formula (1);
X⁰ is H or an organic group having 1 to 30 carbon atoms; and
m′ is an integer of 0 or more.)

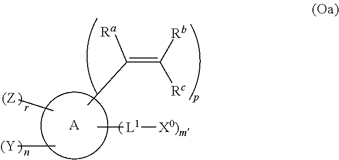

(In the formula (0a),
X⁰, L¹, Y, A, Z, p, m′, n, r, R^{a}, R^{b}, and R^{c} are as defined in the formula (1) and the formula (3a).)

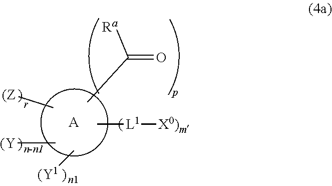

(In the formula (4a),
X⁰, L¹, Y, R^{a}, Z, A, m′, n, p, and r are as defined in the formula (1) and the formula (3a);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y optionally have a substituent; and
n1 is an integer of 1 or more and n or less.)

In step HX, the compounds obtained by introducing a halogen or a group containing a halogen into the compounds represented by the formula (3a), the formula (0a), and the formula (4a) are not particularly limited, and are preferably represented by the following formula (3b), the following formula (4b), and the following formula (0b), respectively.

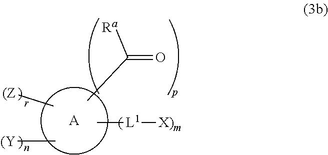

(In the formula (3b),
X, L¹, Y, R^{a}, Z, A, m, n, p, and r are as defined in the formula (1).)

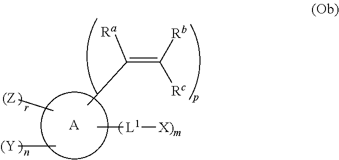

(In the formula (0b),
X, L¹, Y, A, Z, p, m, n, r, R^{a}, R^{b}, and R^{c} are as defined in the formula (1) and the formula (3b).)

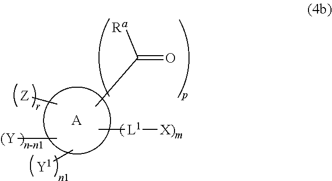

(In the formula (4b),
X, L¹, Y, R^{a}, Z, A, m, n, p, and r are as defined in the formula (1), the formula (3a), and the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y optionally have a substituent; and
n1 is an integer of 1 or more and n or less.)

In the present embodiment, from the viewpoint of achieving the effects by the present invention more effectively and reliably, it is preferable to further comprise a step of halogenating the compound represented by the formula (3a) to produce the compound represented by the formula (3b), a step of halogenating the compound represented by the formula (0a) to produce the compound represented by the formula (0b), and a step of halogenating the compound represented by the formula (4a) to produce the compound represented by the formula (4b), without particular limitation.

### <Step ST>

In the present embodiment, step ST is a step of introducing an unsaturated double bond. Step ST preferably comprises any one of step (W), step (C), and step (D), without particular limitation. Step (W) can utilize, for example, the Wittig reaction, step (C) can utilize, for example, the cinnamic acid decarboxylation reaction, and step (D) can utilize, for example, the dehydration reaction.

### (Step (W))

In the present embodiment, step (W) is preferably a step comprising at least:

W1) a step of providing a carbonyl compound B represented by the following formula (3a) or the following formula (3b), and
W2) a step of forming an unsaturated double bond from the carbonyl moiety of the carbonyl compound B.

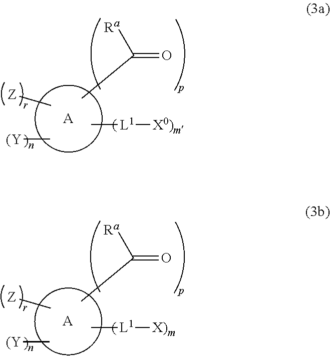

(In the formula (3a) and the formula (3b),
X, L¹, Y, R^{a}, Z, A, m, n, p, and r are as defined in the formula (1);
X⁰ is H or an organic group having 1 to 30 carbon atoms; and
m′ is an integer of 0 or more.)

In step W2 of the present embodiment, it is preferable that a compound represented by the following formula (0a) or the following formula (0b) be obtained by forming an unsaturated double bond from the carbonyl moiety of the carbonyl compound B.

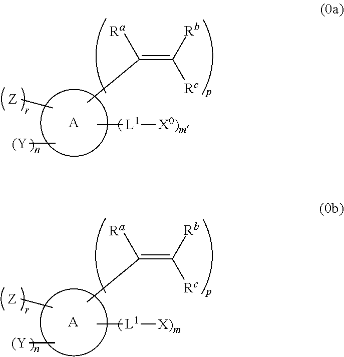

(In the formula (0a) and the formula (0b),
X, X⁰, L¹, Y, A, Z, p, m, m′, n, r, R^{a}, R^{b}, and R^{c} are as defined in the formula (1), the formula (3a), and the formula (3b).

In step (W) of the present embodiment, the term forming an unsaturated double bond from the carbonyl moiety of the carbonyl compound B means converting the carbonyl group to an unsaturated double bond.

In the present embodiment, examples of the carbonyl compound B represented by the formula (3a) or the formula (3b) include, but are not particularly limited to, compounds having the structures given below.

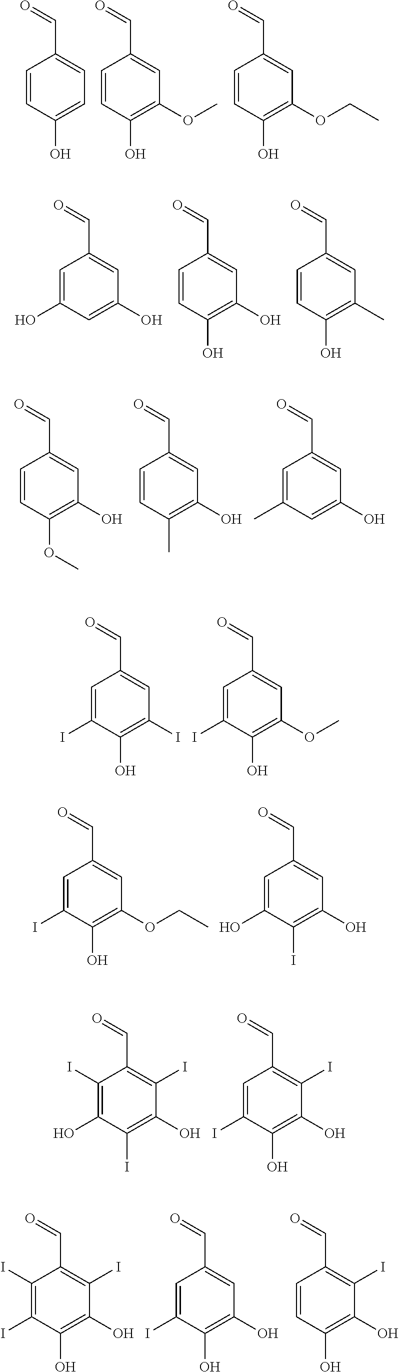

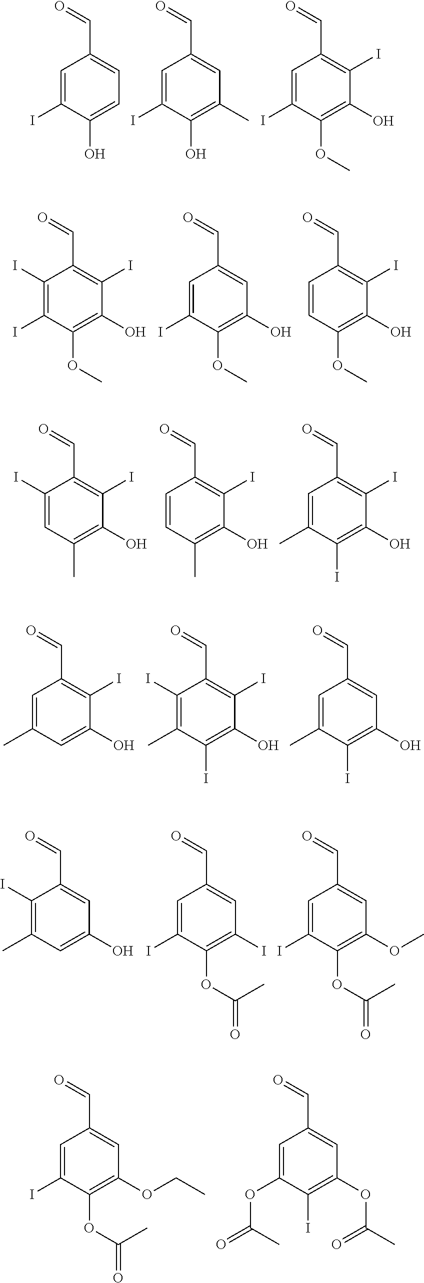

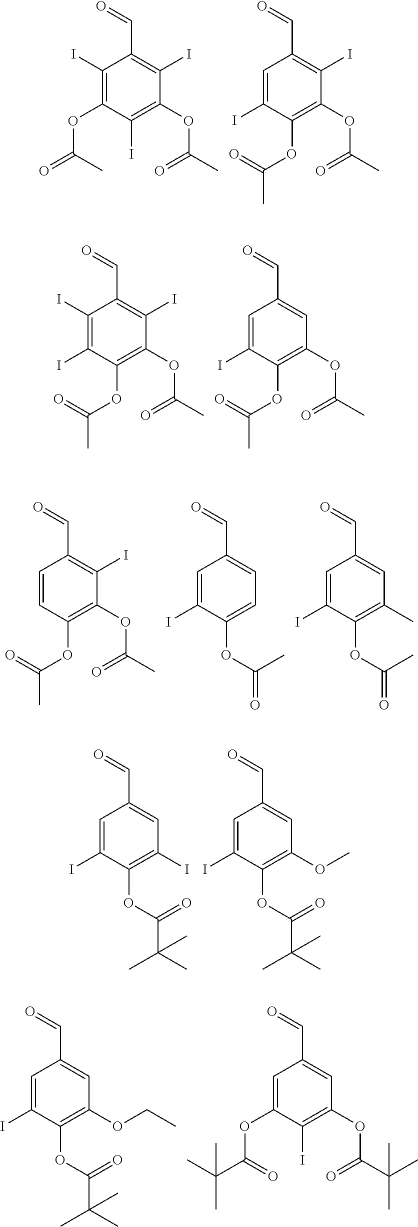

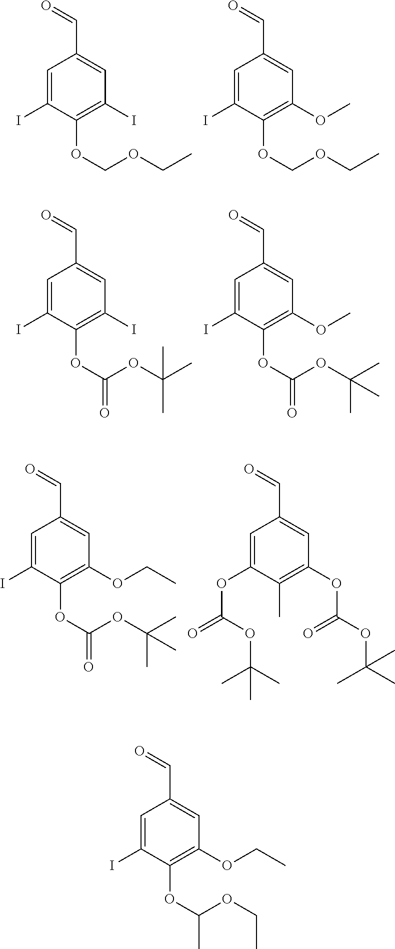

In the formula (3a) or the formula (3b), when the compound A is used as a resist material, A is preferably benzene, toluene, or a heteroaromatic ring, in terms of the effect per mass with respect to the stability of the X group in the resin and the improvement of lithography performance such as the improvement of the sensitivity due to X group, the solubility in a developing solution of the resin when A is incorporated in the resin for lithography as a constitutional unit of the copolymer, and the effect of suppressing partial crystallinity in a resin matrix.

The compound obtained in step (W) of the present embodiment is not particularly limited, as long as it is a compound into which an unsaturated double bond is introduced, and hereinafter, may be referred to as an olefin compound.

Examples of step W1 of the present embodiment include, but are not particularly limited to, a step of providing a carbonyl compound B by a conventionally known method. A commercially available compound may be utilized as the compound B.

In step W2 of the present embodiment, examples of the reaction of forming an unsaturated double bond from the carbonyl moiety of the carbonyl compound B include, but are not particularly limited to, the Wittig reaction.

Examples of the solvent used in step W2 include, but are not particularly limited to, a wide variety of solvents including polar aprotic solvents and protic polar solvents, and a single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used. The solvent is not particularly limited, and is preferably a polar aprotic solvent or a mixture thereof, and more preferably a polar aprotic solvent, from the viewpoint of suppressing by-products. In step W2, the solvent is effective but is not an essential component. Preferred examples of polar aprotic solvents include, but are not particularly limited to, an ether solvent such as diethyl ether, tetrahydrofuran, dimethoxyethane, diglyme, triglyme, cyclopentyl methyl ether, and methyl tert-butyl ether; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, and hexamethylphosphorous triamide; and dimethylsulfoxide. Tetrahydrofuran, acetonitrile, toluene, or dimethylformamide is more preferable. Preferred examples of protic polar solvents include, but are not particularly limited to, water; an alcohol solvent such as methanol, ethanol, propanol, and butanol; di(propylene glycol) methyl ether, di(ethylene glycol) methyl ether, 2-butoxyethanol, ethylene glycol, 2-methoxyethanol, propylene glycol methyl ether, n-hexanol, and n-butanol.

The amount of the solvent used can be arbitrarily set according to, for example, the substrate, catalyst, and reaction conditions to be used, without particular limitation, and is preferably 0 to 10000 parts by mass, and more preferably 100 to 2000 parts by mass, based on 100 parts by mass of the reaction raw materials, from the viewpoint of the yield.

In step W2 of the present embodiment, an organic phosphorus compound may be used. As the organic phosphorus compound, a wide variety of organic phosphorus compounds which function under the reaction conditions in which an unsaturated double bond is formed from the carbonyl moiety of the carbonyl compound B can be used. Preferred examples of organic phosphorus compounds include, but are not particularly limited to, a phosphorane compound such as alkylene triphenylphosphorane, alkylene trimethylphosphorane, alkylene triethylphosphorane, and alkylene tributylphosphorane; a phosphonium compound such as alkyltriphenylphosphonium halide and alkyltributylphosphonium halide; a phosphate ester compound such as dimethyl alkylphosphate, diethyl alkylphosphate, and dibutyl alkylphosphate; a phosphine compound such as trimethylphosphine, triethylphosphine, tributylphosphine, and triphenylphosphine; and phosphite ester such as trimethyl phosphite, triethyl phosphite, and tributyl phosphite. These organic phosphorus compounds may be used as they are, and may be used in combination with a base or halogenated alkyl by a known method to form phosphorus ylide. The organic phosphorus compound is preferably trimethylphosphine, phosphite ester, or phosphonate ester, from the viewpoint of easily removing organic phosphorous by-products generated by the reaction of forming an unsaturated double bond from the carbonyl moiety of the carbonyl compound B, and more preferably alkyltriphenylphosphonium halide, from the viewpoint of the availability of the organic phosphorus compound. In step W2, when the organic phosphorus compound is used, phosphine oxide may be produced as a by-product.

The amount of the organic phosphorus compound used can be arbitrarily set according to, for example, the substrate, organic phosphorus compound, and reaction conditions to be used, without particular limitation, and is preferably 1 to 500 parts by mass, and from the viewpoint of the yield, more preferably 30 to 300 parts by mass, based on 100 parts by mass of the reaction raw materials. The amount of the organic phosphorus compound used can be arbitrarily set according to, for example, the substrate, organic phosphorus compound, and reaction conditions to be used, without particular limitation, and is preferably 1 to 5 times, and more preferably 1.2 to 3 times the amount of the reaction raw materials in a molar ratio, from the viewpoint of reducing a load in the purification process after the reaction.

When the organic phosphorus compound is used in step W2 of the present embodiment, a base may be further used. In step W2, as the base used in combination with the organic phosphorus compound, a wide variety of bases which function under the reaction conditions in which an unsaturated double bond is formed from the carbonyl moiety of the carbonyl compound B can be used. Preferred examples of bases include, but are not particularly limited to, an alkali metal salt of alkoxide, an organic lithium compound, a lithium amide compound, a metal hydride, and a nitrogen-containing organic compound such as a nitrogen-containing heterocyclic compound and a nitrogen-containing alicyclic compound, and specific examples thereof include sodium methoxide, potassium methoxide, sodium ethoxide, potassium ethoxide, sodium tert-butoxide, potassium tert-butoxide, n-butyllithium, sec-butyllithium, tert-butyllithium, phenyllithium, lithium diisopropylamide, lithium tetramethylpiperidide, lithium hydride, sodium hydride, diazabicycloundecene, diazabicyclononene, 1,5,7-triazabicyclo[4.4.0]dec-5-ene, 7-methyl-1,5,7-triazabicyclo[4.4.0]dec-5-ene, and 7-methyl-1,5,7-triazabicyclo[4.4.0]dec-5-ene. Among them, the nitrogen-containing organic compound is preferably an organic base containing no metal atom as a constituent element, further preferably a nitrogen-containing heterocyclic compound or a nitrogen-containing alicyclic compound, and particularly preferably diazabicycloundecene, diazabicyclononene, 1,5,7-triazabicyclo[4.4.0]dec-5-ene, 7-methyl-1,5,7-triazabicyclo[4.4.0]dec-5-ene, and 7-methyl-1,5,7-triazabicyclo[4.4.0]dec-5-ene, without particular limitation, from the viewpoint of suppressing pattern defects in the resist composition produced using the compound A when the compound A is used as a resist material, and achieving both low nucleophilicity and required basicity to improve reaction efficiency and suppress side reactions during production of the compound A.

The amount of the base used can be arbitrarily set according to, for example, the substrate, organic phosphorus compound, base, and reaction conditions to be used, without particular limitation, and is preferably 1 to 500 parts by mass, and from the viewpoint of the yield, more preferably 30 to 300 parts by mass, based on 100 parts by mass of the reaction raw materials.

When the organic phosphorus compound is used in step W2 of the present embodiment, a polymerization inhibitor may be further used. As the polymerization inhibitor, a wide variety of polymerization inhibitors which function under the reaction conditions in which an unsaturated double bond is formed from the carbonyl moiety of the carbonyl compound B can be used. In step W2, the polymerization inhibitor is effective but is not an essential component. Preferred examples of polymerization inhibitors include, but are not particularly limited to, hydroquinone, hydroquinone monomethyl ether (hereinafter, sometimes referred to as methoquinone), 4-tert-butylcatechol, phenothiazine, and an n-oxyl (nitroxide) inhibitor, Prostab® 5415 (bis(1-oxyl-2,2,6,6-tetramethylpiperidin-4-yl)sebacate which is commercially available from Ciba Specialty Chemicals, Tarrytown, NY, CAS #2516-92-9), 4-hydroxy-TEMPO (4-hydroxy-2,2,6,6-tetramethylpiperidin-1-yloxy, which is commercially available from TCI, CAS #2226-96-2), and Uvinul® 4040P (1,6-hexamethylene-bis(N-formyl-N-(1-oxyl-2,2,6,6-tetramethylpiperidin-4-yl)amine) which is commercially available from BASF Corp., Worcester, MA).

The amount of the polymerization inhibitor used can be arbitrarily set according to, for example, the substrate, catalyst, and reaction conditions to be used, without particular limitation, and is preferably 0.0001 to 100 parts by mass, and from the viewpoint of the yield, more preferably 0.001 to 10 parts by mass, based on 100 parts by mass of the reaction raw materials.

When the organic phosphorus compound is used in step W2 of the present embodiment, a polymerization retardant may be further used. As the polymerization retardant, a wide variety of polymerization retardants which function under the reaction conditions in which an unsaturated double bond is formed from the carbonyl moiety of the carbonyl compound B can be used. In step W2, the polymerization retardant is effective but is not an essential component.

When the organic phosphorus compound is used in step W2 of the present embodiment, a polymerization inhibitor may be further used in combination with a polymerization retardant. The polymerization retardant is well-known in the art as a compound that can delay the polymerization reaction, but cannot prevent all polymerization from occurring. Examples of the polymerization retardant include, but are not particularly limited to, an aromatic nitro compound such as dinitro-ortho-cresol (DNOC) and dinitro butyl phenol (DNBP). The method for producing a polymerization retardant is well-known in the art, and examples thereof include the methods described in U.S. Pat. No. 6,339,177; and see Park et. al., Polymer (Korea) (1988), 12(8), 710-19). From the viewpoint of controlling styrene polymerization, examples of the method for using the polymerization retardant include the method described in Bushby et. al., Polymer (1998), 39(22), 5567-5571.

The amount of the polymerization retardant used can be arbitrarily set according to, for example, the substrate, catalyst, and reaction conditions to be used, without particular limitation, and is preferably 0.0001 to 100 parts by mass, and from the viewpoint of the yield, more preferably 0.001 to 10 parts by mass, based on 100 parts by mass of the reaction raw materials.

When the organic phosphorus compound is used in step W2 of the present embodiment, a wide variety of quenching agents which function under the reaction conditions in which an unsaturated double bond is formed from the carbonyl moiety of the carbonyl compound B can be used as the quenching agent. In the present embodiment, the quenching agent means an agent having a function of deactivating phosphorus ylide. In step W2, the quenching agent is effective but is not an essential component.

Examples of the quenching agent include, but are not limited to, ethanol, aqueous ammonium chloride solution, water, hydrochloric acid, and sulfuric acid.

The amount of the quenching agent used can be arbitrarily set according to the amount of the reducing agent to be used, without particular limitation. In general, it is preferably 1 to 500 parts by mass, and from the viewpoint of the yield, more preferably 50 to 200 parts by mass, based on 100 parts by mass of the reducing agent.

When the organic phosphorus compound is used in step W2 of the present embodiment, the reaction temperature is not particularly limited, and is different according to the concentration of the substrate, the stability of the target olefin compound, the selection of the catalyst, and the desired yield. From the viewpoint of suppressing the polymerization of the olefin compound and the removability of phosphine oxide, which will be mentioned later, the reaction temperature is, without particular limitation, preferably −80° C. or more and 80° C. or less, more preferably −50° C. or more and 60° C. or less, further preferably −30° C. or more and 50° C. or less, and particularly preferably −20° C. or more and 30° C. or less. When triphenylphosphine oxide is generated as phosphine oxide, which will be mentioned later, the reaction temperature is preferably −80° C. or more and 80° C. or less.

When the organic phosphorus compound is used in step W2 of the present embodiment, the reaction pressure is not particularly limited, and is different according to the concentration of the substrate, the stability of the target olefin compound, the selection of the catalyst, and the desired yield. The pressure can be adjusted by using an inert gas such as nitrogen, or a suction pump or the like. In the reaction at high pressure, a conventional pressure reaction vessel comprising a shaking vessel, a rocker vessel, a stirred autoclave, and the like can be used, without particular limitation. When triphenylphosphine oxide is generated as phosphine oxide, which will be mentioned later, the preferred reaction pressure is reduced pressure to normal pressure, and normal pressure is preferable.

When the organic phosphorus compound is used in step W2 of the present embodiment, the reaction time is not particularly limited, and is different according to the concentration of the substrate, the stability of the target olefin compound, the selection of the catalyst, and the desired yield. The reaction time is not particularly limited, and is preferably 6 hours or less, and more preferably 15 minutes or more and 600 minutes or less. When triphenylphosphine oxide is generated as phosphine oxide, which will be mentioned later, the reaction time is preferably 15 minutes or more and 600 minutes or less.

As an example of the reaction conditions of step W2 of the present embodiment, the carbonyl compound B represented by the formula (3a) or (3b), a catalyst, and a solvent are added to a reaction vessel to form a reaction mixture. In the present embodiment, the reaction mixture means a mixture containing a starting compound (in step W2, the carbonyl compound B), a target compound (in step W2, a compound in which an unsaturated double bond is formed from the carbonyl moiety of the carbonyl compound B), a reagent required for the reaction, a reaction intermediate, a by-product of the reaction, and the like. The reaction vessel is not particularly limited, and a known reaction vessel can be arbitrarily selected. The reaction may be carried out by arbitrarily selecting a publicly known method such as a batch method, a semi-batch method, or a continuous method.

In step W2, the reaction temperature is not particularly limited, and a preferred range thereof may be different according to the concentration of the substrate, the stability of the target substance formed, the selection of the catalyst, and the desired yield. The reaction temperature is preferably −100° C. or more and 100° C. or less, and from the viewpoint of the yield, more preferably −90° C. or more and 80° C. or less, further preferably −80° C. or more and 60° C. or less, and particularly preferably −80° C. or more and 40° C. or less. When 4-hydroxy-5-iodo-3-methoxybenzaldehyde is used as the carbonyl compound B, the reaction temperature is preferably −80° C. or more and 80° C. or less.

In step W2, the reaction pressure is not particularly limited, and a preferred range thereof may be different according to the concentration of the substrate, the stability of the target compound formed, the selection of the catalyst, and the desired yield. The reaction pressure is not particularly limited, and can be adjusted by using an inert gas such as nitrogen, or a suction pump or the like. In the reaction at high pressure, a conventional pressure reaction vessel comprising a shaking vessel, a rocker vessel, a stirred autoclave, and the like may be used, without particular limitation. When 4-hydroxy-5-iodo-3-methoxybenzaldehyde is used as the carbonyl compound B, the reaction pressure is preferably reduced pressure to normal pressure, and more preferably reduced pressure.

In step W2, the reaction time is not particularly limited, and a preferred range thereof is different according to the concentration of the substrate, the stability of the product formed, the selection of the catalyst, and the desired yield. The reaction time is preferably 6 hours or less, and more preferably 15 minutes or more and 600 minutes or less. When 4-hydroxy-5-iodo-3-methoxybenzaldehyde is used as the carbonyl compound B, the reaction time is preferably 15 minutes or more and 600 minutes or less.

After an unsaturated double bond is formed from the carbonyl moiety of the carbonyl compound B, step W2 may further comprise a step of isolating or/and purifying the target compound. Isolation and/or purification can be conducted using a conventionally known suitable method after the termination of the reaction of forming an unsaturated double bond from the carbonyl moiety of the carbonyl compound B. Examples thereof include, but are not particularly limited to, a method in which the reaction mixture is poured onto ice water and extracted in a solvent, such as an acetate ester solvent such as ethyl acetate and butyl acetate, or an ether solvent such as toluene, cyclopentyl methyl ether, or diethyl ether, and the solvent is then removed using evaporation under reduced pressure to recover the product. A high purity target compound may be isolated and purified by a separation and purification method by filtration, concentration, distillation, extraction, crystallization, recrystallization, column chromatography, activated carbon, or the like, which are well-known purification methods in the art, or a combined method thereof.

When the organic phosphorus compound is used in step W2 of the present embodiment, a step of removing phosphine oxide generated may be further included. Hereinafter, the Wittig reaction will be specifically described as the case of using an organic phosphorus compound in step W2. Example of the method for removing phosphine oxide generated by the Wittig reaction include a method for removing phosphine oxide using an adsorbent, and a method in which phosphine oxide generated is solidified and the solidified phosphine oxide is removed by solid-liquid separation. Among these steps, only a single step may be used, or a plurality of steps may be used in combination. The method is arbitrarily selected according to the stability, reactivity, and solubility of the target compound, the stability, reactivity, and solubility of phosphine oxide, impurities produced as by-products, the desired yield, and the like.

In the present embodiment, the method for removing phosphine oxide using an adsorbent is preferable, from the viewpoint of selectivity reducing phosphine oxide without impairing the yield of the target compound, and being capable of removing high molecular weight impurities generated in the production process or the storage of the intermediate simultaneously.

Examples of the adsorbent include, but are not particularly limited to, silica gel, modified silica gel, activated charcoal, activated alumina, zeolite, hydrotalcite, florisil, activated clay, diatomaceous earth, a synthetic adsorbent, and an ion exchange resin. Examples of the modified silica gel include sulfuric acid on silica gel. Among them, silica gel, zeolite, activated alumina, an ion exchange resin, or modified silica gel is preferable, silica gel, modified silica gel, or an ion exchange resin is more preferable, and silica gel is further preferable, from the viewpoint of effectively adsorbing polar groups contained in phosphine oxide and high molecular weight impurities.

Examples of the method for using the adsorbent include a method in which the adsorbent is put in a solution containing the target olefin compound generated by the Wittig reaction and phosphine oxide and stirred, and the adsorbent is then filtered off, and a method in which a solution containing the olefin compound and phosphine oxide is passed through a container filled with the adsorbent, a column, a filter, or the like. From the viewpoint of production, a method in which the adsorbent is added and stirred is preferable.

The amount of the adsorbent used can be appropriately set according to the adsorbent to be used, phosphine oxide, the olefin compound, the solvent, the temperature, the desired yield, and the like, without particular limitation. From the viewpoint of efficiently removing phosphine oxide, the amount of the adsorbent used is preferably as high as possible, and from the viewpoint of suppressing the reduction of the yield of the olefin compound, the amount of the adsorbent used is preferably as low as possible. The amount of the adsorbent used is preferably 0.01 to 100 parts by mass, more preferably 0.1 to 50 parts by mass, and further preferably 0.3 to 20 parts by mass, based on 1 part by mass of the olefin compound of the target compound.

In the method for removing phosphine oxide using an adsorbent, the solvent is not particularly limited, and a wide variety of solvents including polar aprotic solvents and protic polar solvents can be used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used. Preferred examples of polar aprotic solvents include, but are not particularly limited to, an ether solvent such as diethyl ether, tetrahydrofuran, dimethoxyethane, diglyme, triglyme, cyclopentyl methyl ether and methyl tert-butyl ether; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, and hexamethylphosphorous triamide; and dimethylsulfoxide. Dimethylsulfoxide is more preferable. Preferred examples of protic polar solvents include, but are not particularly limited to, water; an alcohol solvent such as methanol, ethanol, propanol, and butanol; di(propylene glycol) methyl ether, di(ethylene glycol) methyl ether, 2-butoxyethanol, ethylene glycol, 2-methoxyethanol, propylene glycol methyl ether, n-hexanol, and n-butanol. From the viewpoint of effectively adsorbing polar groups contained in phosphine oxide and high molecular weight impurities, a hydrocarbon solvent such as toluene and hexane is more preferable.

In the method for removing phosphine oxide using an adsorbent, the temperature is not particularly limited, and a preferred range thereof is different according to the adsorbent, the concentration of the target olefin compound, the concentration of phosphine oxide, the content of high molecular weight impurities, the stability of the olefin compound, and the desired yield. The reaction temperature is not particularly limited, and is preferably −80° C. or more and 80° C. or less, more preferably −50° C. or more and 60° C. or less, further preferably −30° C. or more and 50° C. or less, and particularly preferably −20° C. or more and 30° C. or less, from the viewpoint of suppressing the polymerization of the olefin compound and the removability of phosphine oxide. When silica gel is used as the adsorbent, the temperature range is preferably −80° C. or more and 80° C. or less.

In the present embodiment, examples of the method in which phosphine oxide generated is solidified by the Wittig reaction and the solidified phosphine oxide is removed by solid-liquid separation include a method in which an acid is further added to solidify phosphine oxide as a composite with the acid and the solidified phosphine oxide is removed by solid-liquid separation, and a method in which phosphine oxide is solidified by changing the solvent system and the solidified phosphine oxide is removed by solid-liquid separation.

In the method in which an acid is further added to solidify phosphine oxide as a composite with the acid, a wide variety of acids which function under the reaction conditions in which an unsaturated double bond is formed from the carbonyl moiety of the carbonyl compound B is used, without particular limitation. Bronsted acid is preferably used as the acid, without particular limitation. The reason why use of Bronsted acid is preferable is considered by the present inventors as follows, but is not particularly limited. That is, use of Bronsted acid enables use of an organic solvent other than the polar solvent, so that the amount of the acid used can be suppressed to about the same amount as that of phosphine oxide and a highly purified target olefin compound can be achieved. In addition, since the operation of removing phosphine oxide is only filtration, the target olefin compound can be recovered in a high yield and the operation can be conducted in an industrially applicable process.

In the method in which an acid is further added to solidify phosphine oxide as a composite with the acid, examples of the acid include, but are not particularly limited to, an inorganic acid such as hydrochloric acid, sulfuric acid, phosphoric acid, hydrobromic acid, and hydrofluoric acid; an organic acid such as acetic acid, oxalic acid, malonic acid, succinic acid, adipic acid, sebacic acid, citric acid, fumaric acid, maleic acid, formic acid, benzoic acid, salicylic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; an alcohol such as hexafluoroisopropanol, benzylalcohol, diphenylmethanol, triphenylmethanol, and methanol; and a phenol such as phenol, o-cresol, m-cresol, p-cresol, catechol, resorcinol, hydroquinone, salicyl alcohol, bisphenol A, 1-naphthol, and 2-naphthol. These acids can be used singly, or two or more thereof can also be used in combination. These acids may be used without dilution or may be used by being diluted with water or an organic solvent. From the viewpoint of the removability of phosphine oxide, the acid is preferably added without dilution or diluted with an organic solvent and then added, and from the viewpoint of filtration properties, the acid is further preferably diluted with an organic solvent and then added. The organic solvent for diluting the acid is not particularly limited, as long as it is a solvent that uniformly dissolves or disperses the acid, and from the viewpoint of production, a solvent that mixes with a Wittig reaction solution is preferable. As the organic solvent for diluting the acid, for example, a hydrophilic organic solvent such as an ether, e.g., THF and dioxane, and an alcohol, e.g., methanol, can be used, from the viewpoint of using an acid having a high polarity and a nonpolar organic solvent (Wittig reaction solution).

The pKₐ of the acid is not particularly limited, and is preferably 11 or less, more preferably 9 or less, and further preferably 6 or less, from the viewpoint of the removability of phosphine oxide. When an acid having a high molecular weight is used, phosphine oxide can also be effectively removed regardless of pKₐ. In this case, the molecular weight of the acid is not particularly limited, and is preferably 70 or more, more preferably 80 or more, and further preferably 90 or more.

The amount of the acid used can be appropriately set according to phosphine oxide, the target olefin compound, the acid, the reaction conditions, and the like, without particular limitation. From the viewpoint of the removability of phosphine oxide, the amount of the acid used is preferably 0.01 to 100 parts by mass based on 100 parts by mass of phosphine oxide. From the viewpoint of the removability of phosphine oxide, the amount of the acid used is preferably as high as possible, and from the viewpoint of suppressing the polymerization of the olefin compound, it is preferably as low as possible.

In the method in which an acid is further added to solidify phosphine oxide as a composite with the acid, from the viewpoint of the balance between the removability of phosphine oxide and suppression of the polymerization of the olefin compound, the amount of the acid used preferably satisfies the following expression 1.

$$0.5 \leq \frac{\sum ( Amount of substance of the acid ) \times ( Valence of the acid )}{( Amount of substance of the phosphine oxide )} \leq 5$$

When a plurality of acids is used or an acid is added portionwise, (amount of substance of the acid)×(valence of the acid) in Expression 1 is added by Σ. When the target olefin compound may act as the above acid, for example, the target olefin compound has a phenol skeleton, (amount of substance of the acid)×(valence of the acid) of the olefin compound is not added by Σ in Expression 1.

In the method in which an acid is further added to solidify phosphine oxide as a composite with the acid, an acid may be directly added to the Wittig reaction solution, or an acid may be added after the solvent of the Wittig reaction solution is changed by an operation such as filtration, concentration, distillation, extraction, back extraction into an aqueous layer, crystallization, or recrystallization well known in the art or a combined method thereof.

In the method in which an acid is further added to solidify phosphine oxide as a composite with the acid, the SP value of the solvent contained in the Wittig reaction solution when the acid is added is preferably 30.0 MPa^{1/2} or less, more preferably 25.0 MPa^{1/2} or less, further preferably 23.0 MPa^{1/2} or less, and particularly preferably 20.0 MPa^{1/2} or less, from the viewpoint of the removability of phosphine oxide. The SP value is a value calculated by “HSPiP 4th Edition 4.1.03”.

In the method in which an acid is further added to solidify phosphine oxide as a composite with the acid, the temperature when the acid is added and the reaction temperature for forming the composite of phosphine oxide and the acid are not particularly limited, and are different according to the concentration of the substrate, the stability of the target olefin compound, the selection of the catalyst, and the desired yield. The temperature is preferably −80° C. or more and 80° C. or less, more preferably −50° C. or more and 60° C. or less, further preferably −30° C. or more and 50° C. or less, and particularly preferably −20° C. or more and 30° C. or less, from the viewpoint of suppressing the polymerization of the olefin compound and the removability of phosphine oxide. When triphenylphosphine oxide is generated as phosphine oxide, the temperature is preferably −80° C. or more and 80° C. or less.

In the method in which an acid is further added to solidify phosphine oxide as a composite with the acid, the pressure when the acid is added and the reaction temperature for forming the composite of phosphine oxide and the acid are not particularly limited, and are different according to the concentration of the substrate, the stability of the target olefin compound, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without particular limitation. When triphenylphosphine oxide is generated as phosphine oxide, the pressure upon addition of the acid and the reaction pressure upon formation of the composite of phosphine oxide with the acid are preferably reduced pressure to normal pressure, and more preferably normal pressure.

In the method in which an acid is further added to solidify phosphine oxide as a composite with the acid, the reaction time for forming the composite of phosphine oxide and the acid is not particularly limited, and are different according to the concentration of the substrate, the stability of the target olefin compound, the selection of the catalyst, and the desired yield. The reaction time is preferably 6 hours or less, and more preferably 15 minutes or more and 600 minutes or less. When triphenylphosphine oxide is generated as phosphine oxide, the reaction time is preferably 15 minutes or more and 600 minutes or less.

The phosphine oxide solidified as the composite with the acid is preferably removed by solid-liquid separation. The method for solid-liquid separation is not particularly limited, and is different according to the solid concentration in the slurry, the density distribution, the particle size distribution, the temperature, the solvent, the viscosity, the specific gravity, the selection of phosphine oxide, the stability of the composite, the concentration of the olefin compound, the stability of the olefin compound, and the desired yield. Conventional solid-liquid separators including a vacuum filter, a pressure filter, a press filter, and a centrifugal filter can be used.

In step W2 of the present embodiment, examples of the method for solidifying phosphine oxide generated by the Wittig reaction and removing the solidified phosphine oxide by solid-liquid separation include a method in which phosphine oxide is solidified by changing the solvent system and the solidified phosphine oxide is removed by solid-liquid separation.

In step W2, as the method for changing the solvent system, a known method can be arbitrarily used. To change the solvent system, a method such as the addition of a solvent component, concentration, distillation, extraction, or back extraction into an aqueous layer can be used. All components contained in the reaction solution may be taken out once by a method such as concentration to dryness, salting-out, crystallization, or recrystallization, and then the solvent may be changed to an arbitrary solvent system.

In step W2 of the present embodiment, when phosphine oxide is solidified by changing the solvent system and the phosphine oxide is removed by solid-liquid separation, the carbonyl compound B is preferably a compound represented by the following formula (4a) or the following formula (4b), from the viewpoint of the removability of phosphine oxide. In this case, the compound formed by using an organic phosphorus compound in step W2 is preferably a compound represented by the following formula (5a) or the following formula (5b).

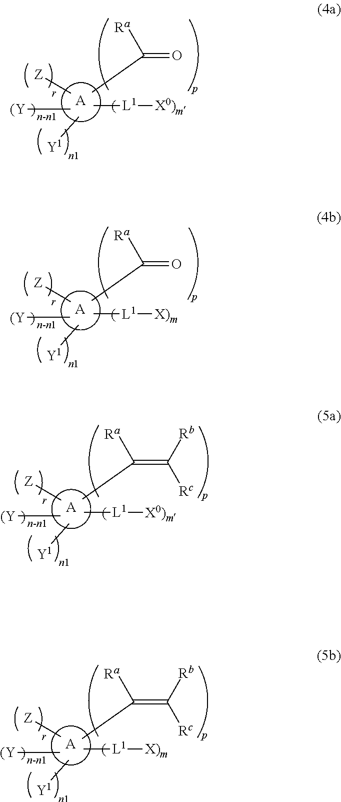

(In the formula (4a), the formula (4b), the formula (5a), and the formula (5b),
X, X⁰, L¹, Y, R^{a}, R^{b}, R^{c}, Z, A, m, m′, n, p, and r are as defined in the formula (1), the formula (3a), and the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally has a substituent; and
n1 is an integer of 1 or more and n or less.)

In the present embodiment, from the viewpoint of the removability of phosphine oxide, the reason why the carbonyl compound B is preferably a compound represented by the formula (4a) or the formula (4b) is considered by the present inventors as follows, but is not particularly limited. That is, since phosphine oxide has a partial structure having a high polarity, the solubility of phosphine oxide in a solvent can be reduced by controlling the hydrophilicity and hydrophobicity and/or polarity of the solvent in the reaction solution, so that phosphine oxide can be solidified and removed by solid-liquid separation. Either a method for changing the solvent system to a solvent system having a higher polarity than that of phosphine oxide to solidify phosphine oxide or a method for changing the solvent system to a solvent system having a lower polarity than that of phosphine oxide to solidify phosphine oxide can be applied. From the viewpoint of quality maintenance such as mass productivity and impurity control, a method in which the compound represented by the formula (5a) or the formula (5b) is obtained in a state dissolved in the solvent is preferable, and a method for changing the solvent system to a solvent system having a lower polarity than that of phosphine oxide to solidify phosphine oxide is more preferable. By changing the solvent system to a solvent system having a lower polarity than that of phosphine oxide, the effect of the polymerization inhibitor, which will be mentioned later, can be maintained in step (W), and the effect of suppressing the quality deterioration due to the side reaction of the polymerization of the compound represented by the formula (5a) or the formula (5b) can be maintained.

When the carbonyl compound B is a compound represented by the formula (4a) or the formula (4b), examples of the method for changing the solvent system include a method in which the reaction solvent used in the Wittig reaction is removed by concentration under reduced pressure, and a solvent suitable for solidifying phosphine oxide is added. From the viewpoint of the removability of phosphine oxide, a method for changing the solvent system to a solvent system having a lower polarity than that of phosphine oxide to solidify phosphine oxide is preferable.

Further, the compound represented by the formula (5a) or the formula (5b) preferably has a relationship of hydrophilicity and hydrophobicity with phosphine oxide within a predetermined range, from the viewpoint of solidifying phosphine oxide by changing the solvent system and efficiently proceeding removal by solid-liquid separation. Examples of the relationship of hydrophilicity and hydrophobicity include, but are not particularly limited to, setting a difference in hydrophilicity and hydrophobicity between the compound represented by the formula (5a) or the formula (5b) and phosphine oxide to a desired range by the HSP distance calculated by “HSPiP 4th Edition 4.1.03”. The HSP distance between the compound represented by the formula (5a) or the formula (5b) and the phosphine oxide is preferably 6.5 or more, and more preferably 7.0 or more.

In step W2, examples of the solvent used when removing phosphine oxide include, but are not particularly limited to, a wide variety of solvents including polar aprotic solvents and protic polar solvents, and a single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used. In step W2, the solvent used when removing phosphine oxide is more preferably a solvent that solidifies phosphine oxide and dissolves the compound represented by the formula (5a) or the formula (5b).

Preferred examples of polar aprotic solvents include, but are not particularly limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene, hexane, and heptane; an amide solvent such as N,N-dimethylformamide, 1-methyl-2-pyrrolidinone, N,N-dimethylacetamide, hexamethylphosphoramide, and hexamethylphosphorous triamide; and dimethylsulfoxide. Preferred examples of protic polar solvents include, but are not particularly limited to, water; an alcohol solvent such as methanol, ethanol, propanol, and butanol; di(propylene glycol) methyl ether, di(ethylene glycol) methyl ether, 2-butoxyethanol, ethylene glycol, 2-methoxyethanol, propylene glycol methyl ether, n-hexanol, and n-butanol. Among them, a combination of carrying out the Wittig reaction in an ether solvent and then changing the solvent to a hydrocarbon solvent is preferable, from the viewpoint of the efficiency of solidifying phosphine oxide and removing phosphine oxide by solid-liquid separation.

In the present embodiment, from the viewpoint of solidifying phosphine oxide by changing the solvent system and removing phosphine oxide by solid-liquid separation, it is preferable to produce a compound represented by the formula (0a) or the formula (0b), or a compound represented by the formula (5a) or the formula (5b), and more preferable to produce a compound represented by the formula (5a) or the formula (5b) in step W2. From the viewpoint of solidifying phosphine oxide by changing the solvent system and removing phosphine oxide by solid-liquid separation, when it is preferable to produce a compound represented by the formula (5a) or the formula (5b) in step W2, the compound represented by the formula (0a) or the formula (0b) may be formed from the compound represented by the formula (5a) or the formula (5b) by various conversion, after the compound represented by the formula (5a) or the formula (5b) is obtained. From the viewpoint of the stability of the target olefin compound and the removability of phosphine oxide in the method for removing phosphine oxide, a step of obtaining the compound represented by the formula (0a) or the formula (0b) from the compound represented by the formula (5a) or the formula (5b) can be arbitrarily selected.

The method for obtaining the compound represented by the formula (0a) or the formula (0b) from the compound represented by the formula (5a) or the formula (5b) is not particularly limited, and for example, the compound represented by the formula (0a) or the formula (0b) can be formed by carrying out the hydrolysis reaction of the alkoxy group, the ester group, the acetal group, the carboxyalkoxy group, the carbonate ester group, the ether group, the thioether group, or the like of at least a part of Y¹ of the formula (5a) and the formula (5b) by using a base, an acid, heat, or the like. In the hydrolysis reaction, a known one can be used as the acid or the base. Examples of the method for treating the acid or the base include, but are not particularly limited to, a method for directly adding the acid or the base into a solution and a method for treating the acid or the base in a multiphase mixture system by liquid separation.

In step W2, when phosphine oxide is solidified by changing the solvent system, a polymerization inhibitor or the like may be used as a stabilizing agent, from the viewpoint of improving the stability of the compound represented by the formula (5a) or the formula (5b) or the compound represented by the formula (0a) or the formula (0b). The polymerization inhibitor is not particularly limited, and polymerization inhibitors as described above can be used. The amount used when the polymerization inhibitor is used is preferably 1 ppm or more and 10000 ppm or less based on the compound represented by the formula (5a) or the formula (5b) or the compound represented by the formula (0a) or the formula (0b). When the amount of the polymerization inhibitor used is 1 ppm or more, the effect of improving the stability of the compound represented by the formula (5a) or the formula (5b) or the compound represented by the formula (0a) or the formula (0b) can be sufficiently obtained. When the amount used is higher than 10000 ppm, the removability of phosphine oxide tends to be reduced by the interaction between the polymerization inhibitor and phosphine oxide. In addition, for example, the residual amount of the polymerization inhibitor remained in the compound represented by the formula (1) obtained by the production method of the present embodiment increases, and when a polymer is produced by using the compound represented by the formula (1), a desired polymer cannot be obtained due to the effect of suppressing polymerization by the polymerization inhibitor incorporated, which are problematic.

### (Step (C))

In the present embodiment, step (C) is preferably a step at least comprising:

C1) a step of providing the carbonyl compound B,
C2) a step of obtaining a compound represented by the following formula (SA2a) or the following formula (SA2b) by using the carbonyl compound B and a compound represented by the following formula (RM1) or malononitrile, and
C3) a step of forming a compound represented by the following formula (0a) or the following formula (0b) by using the compound represented by the following formula (SA2a) or the following formula (SA2b) and a fluoride source.

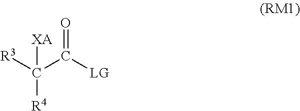

(In the formula (RM1),
LG is a group selected from a hydroxy group, an alkoxy group, a carbonate ester group, an acetal group, and a carboxyl group, and the alkoxy group, the carbonate ester group, the acetal group, and the carboxyl group contain an aliphatic group or aromatic group having 1 to 60 carbon atoms and optionally having a substituent;
R³ is a hydrogen group, or a carboxyl group or ester group having 1 to 60 carbon atoms and optionally having a substituent;
R⁴ is a hydrogen group; and
XA is a group selected from a hydrogen group and a halogen group.)

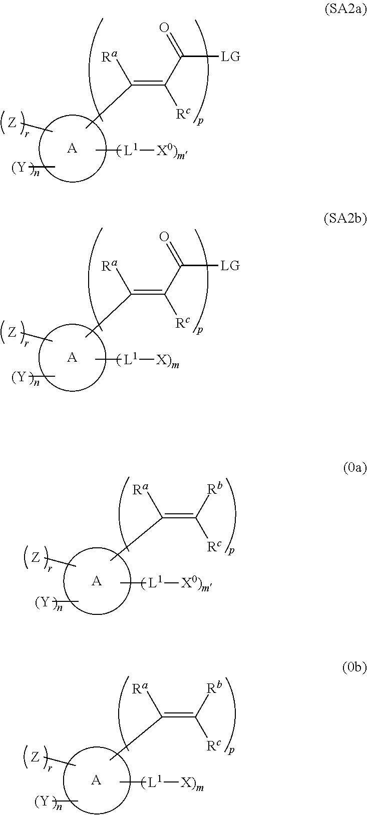

(In the formula (SA2a), the formula (SA2b), the formula (0a), and the formula (0b),
X, X⁰, L¹, Y, A, Z, p, m, m′, n, r, R^{a}, R^{b}, R^{c}, and LG are as defined in the formula (1), the formula (3a), the formula (3b), and the formula (RM1).)

In the present embodiment, step C1 may be the same step as step W1.

In step C2 of the present embodiment, examples of the compound represented by the formula (RM1) include, but are not particularly limited to, a maleate derivative such as maleic acid, dimethyl maleate, diethyl maleate, dipropyl maleate, diisopropyl maleate, and maleic anhydride; and an acetate derivative such as ethyl acetate, propyl acetate, butyl acetate, ethyl achloroacetate, propyl achloroacetate, and butyl achloroacetate. The compound represented by the formula (RM1) is preferably a compound selected from the group consisting of a malonate derivative such as malonic acid, dimethyl malonate, or diethyl malonate; an acetic acid derivative; and an acetate derivative.

In step C2, a method generally employed as a Knoevenagel reaction or a Doebner reaction may be used without particular limitation, and, for example, the conditions described in Journal of Molecular Catalysis B:Enzymatic, 82, 92-95; 2012, Tetrahedron Letters, 46(40), 6893-6896; 2005, and the like may be used. Specifically, the compound represented by the formula (SA2a) or the formula (SA2b) can be obtained by reacting the compound represented by the formula (RM1) or malononitrile with a base in a solvent. In addition to a base, an acid may also be used in combination.

As the base, various known compounds may be used, and examples thereof include, but are not particularly limited to, a nitrogen-containing cyclic compound containing a structure such as pyridine, piperidine, pyrrolidine, azole, diazole, triazole, and morpholine; and a nitrogen-containing compound of a tertiary amine, such as tributylamine, trimethylamine, and trihydroxyethylamine.

Examples of the acid that can be used in combination with the base include, but are not particularly limited to, a weak acid such as acetic acid and propionic acid.

When the acid is used in combination with the base, the balance between acidity and basicity is not particularly limited. When a compound in which m or m′ is 1 or more in the formula (0a) or the formula (0b) is the target compound, the reaction is preferably carried out under acidic conditions.

In step C2, when LG is an alkoxy group, a carbonate ester group, an acetal group, or a carboxyl group in the formula (RM1), it is preferable to further comprise a step of obtaining a compound represented by the formula (SA3) by a reaction of converting LG to a hydroxy group by hydrolysis or the like. The treatment such as hydrolysis is not particularly limited as long as LG can be converted to a hydroxy group, and as one example of the reaction conditions, for example, a deprotection reaction can be carried out by using an acid such as hydrochloric acid, sulfuric acid, and paratoluenesulfonic acid as a catalyst in combination, under temperature conditions such as reflux. As another example of the reaction conditions, a deprotection reaction can be carried out by refluxing using an inorganic base such as sodium hydroxide and potassium hydrate or an organic base such as tertiary amine as the base under solvent conditions such as toluene and xylene.

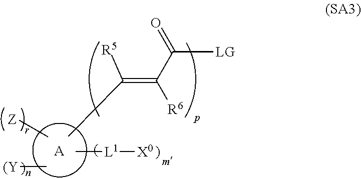

(In the formula (SA3),
LG is a hydroxy group;
X⁰, L¹, Y, A, Z, p, m′, n, and r are as defined in the formula (SA2a) and the formula (0a); and
each of R⁵ and R⁶ is independently H, F, Cl, Br, or an organic group having 1 to 60 carbon atoms and optionally having a substituent.)

In step C2, the compound represented by the formula (SA2a) or the formula (SA2b) may be obtained by further using a reducing agent. When the compound represented by the formula (SA2a) or the formula (SA2b) is obtained by using a reducing agent, the compound represented by the formula (RM1) having higher stability can be used, which is advantageous in terms of conversion and purity. As the reducing agent, various conventionally known materials can be used.

As the reducing agent, a wide variety of reducing agents which function under the reaction conditions for obtaining the compound represented by the formula (SA2a) or the formula (SA2b) is used. Preferred examples of reducing agents include, but are not particularly limited to, a metal hydride and a metal hydride complex compound. Specific examples include, but are not particularly limited to, borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the reducing agent used can be arbitrarily set according to, for example, the substrate, reducing agent, and reaction conditions to be used, and is preferably 1 to 500 parts by mass, and from the viewpoint of the yield, more preferably 10 to 200 parts by mass based on 100 parts by mass of the reaction raw materials, without particular limitation.

Step C3 of the present embodiment is preferably a step of carrying out decarboxylation of the carboxyl group of the compound represented by the formula (SA2a), the formula (SA2b), or the formula (SA3) by using the compound represented by the formula (SA2a), the formula (SA2b), or the formula (SA3), and a fluoride source.

In step C3 of the present embodiment, various solvents can be used as the reaction solvent. The reaction solvent is not particularly limited, as long as it is a solvent that can dissolve the compound represented by the formula (SA2a), the formula (SA2b), or the formula (SA3), and examples thereof include an alcohol solvent such as methanol, ethanol, propanol, and butanol; a ketone solvent such as cyclohexanone, cyclopentanone, MEK, and MIBK; a linear or cyclic ester solvent such as ethyl acetate, butyl acetate, ethyl propionate, isobutyl propionate, ethyl lactate, and gamma butyrolactone; an ether solvent such as diethyl ether; a glycol solvent such as diethylene glycol, PGMEA, and PGME; an aromatic solvent such as toluene and benzene; an amide solvent such as DMF; and water.

In step C3, the reaction solvent is preferably a solvent having a high polarity, having a high dielectric constant, or/and having a low hydrophobicity, from the viewpoint of the solubility of the compound represented by the formula (SA2a), the formula (SA2b), or the formula (SA3), and the fluoride source, and improving the stability of a reaction product. In addition, the reaction solvent is preferably a solvent having a high polarity, from the viewpoint of solubility, and a solvent having a low nucleophilicity, from the viewpoint of stability. For example, the reaction solvent is preferably a cyclic ester such as γbutyrolactone; an ether such as diglyme, dioxane, tetrahydrofuran, and cyclopentyl methyl ether; an amide such as dimethylformamide; or an aromatic hydrocarbon such as toluene, more preferably one or more solvents selected from the group consisting of dimethylsulfoxide, γbutyrolactone, dimethylformamide, dioxane, cyclopentyl methyl ether, toluene, and diglyme, and further preferably one or more solvents selected from the group consisting of γbutyrolactone, dimethylformamide, dioxane, cyclopentyl methyl ether, toluene, and diglyme.

As the fluoride source, various compounds that generate fluorides can be used without particular limitation. Examples thereof include salts of quaternary amines and fluorides, such as tetrabutylammonium fluoride, tetramethylamine fluoride, and tetrahydroxyethylamine fluoride; salts of metal cation species such as tetramethylaluminum and fluorides; salts of phosphonium such as tetraoctadecylphosphonium and fluorides; and fluoride salts of alkali metals such as KF and NaF.

In step C3, the compound represented by the formula (0a) or the formula (0b) is preferably obtained by subjecting the compound represented by the formula (SA2a), the formula (SA2b), or the formula (SA3) to a decarboxylation reaction using a fluoride source at a reaction temperature of 100° C. or less. In the formula (SA2a), the formula (SA2b), or the formula (SA3), when there is concern that the selection of A, Z, Y, L¹, and X may cause modification and decomposition at high temperatures, the reaction temperature is more preferably 80° C. or less, further preferably 60° C. or less, and particularly preferably 50° C. or less.

A polymerization inhibitor may be further used in step C3, and a commonly available commercial product may be used. Examples thereof include, but are not particularly limited to, a nitroso compound such as 2,2,6,6-tetramethyl-4-hydroxypiperidine-1-oxyl, N-nitrosophenylhydroxylamine ammonium salt, N-nitrosophenylhydroxylamine aluminum salt, N-nitroso-N-(1-naphthyl)hydroxylamine ammonium salt, N-nitrosodiphenylamine, N-nitroso-N-methylaniline, nitrosonaphthol, p-nitrosophenol, and N,N′-dimethyl-p-nitrosoaniline; a sulfur containing compound such as phenothiazine, methylene blue, and 2-mercaptobenzimidazole; an amine such as N,N′-diphenyl-p-phenylenediamine, N-phenyl-N′-isopropyl-p-phenylenediamine, 4-hydroxydiphenylamine, and aminophenol; a quinone such as hydroxyquinoline, hydroquinone, methylhydroquinone, p-benzoquinone, and hydroquinone monomethyl ether; a phenol such as p-methoxyphenol, 2,4-dimethyl-6-t-butylphenol, catechol, 3-s-butylcatechol, 2,2-methylenebis-(6-t-butyl-4-methylphenol); an imide such as N-hydroxyphthalimide; an oxime such as cyclohexane oxime, p-quinone dioxime; and dialkyl thiodipropionate. The amount added is preferably 0.001 to 10 parts by mass, and more preferably 0.01 to 1 parts by mass based on 100 parts by mass of the compound represented by the formula (SA2a), the formula (SA2b), or the formula (SA3).

### (Step (D))

In the present embodiment, step (D) is preferably a step comprising at least: D1) a step of providing a compound represented by the formula (1-1a) or the formula (1-1b):

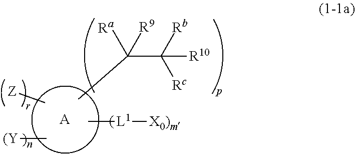

wherein
X⁰, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{c} is an organic group having 1 to 30 carbon atoms and optionally having a substituent, and

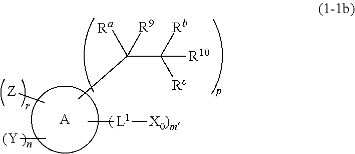

wherein
X, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{c} is an organic group having 1 to 30 carbon atoms and optionally having a substituent, and
   D2) a step of obtaining a compound represented by the following formula (0a) or the following formula (0b) from the compound represented by the formula (1-1a) or the formula (1-1b) by a dehydration reaction using an acid as a catalyst:

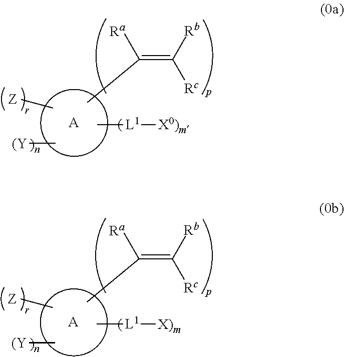

wherein
X, X⁰, L¹, Y, A, Z, p, m, m′, n, r, R^{a}, R^{b}, and R^{c} are as defined in the formula (1), the formula (3a), and the formula (3b).

In step D2 of the present embodiment, a wide variety of reaction solvents including polar aprotic solvents and protic polar solvents can be used as the reaction solvent, without particular limitation, and a single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The reaction solvent is effective, but is not an essential component.

From the viewpoint of improving the yield, preferred examples of polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; dimethylsulfoxide, and sulfolane.

From the viewpoint of improving the yield, the polar aprotic solvent is preferably a solvent having a high relative permittivity, and N,N-dimethylformamide, dimethylsulfoxide, or sulfolane is preferable. The relative permittivity is preferably a relative permittivity in a room temperature area at about 20 to 30° C. of 20 or more. The relative permittivity of the solvent having a high relative permittivity is more preferably 30 or more, and further preferably 40 or more.

From the viewpoint of improving the yield, preferred examples of protic polar solvents include, but are not particularly limited to, water; an alcohol solvent such as methanol, ethanol, propanol, and butanol; di(propylene glycol) methyl ether, cyclopentyl methyl ether, di(ethylene glycol) methylether, 2-butoxyethanol, ethylene glycol, 2-methoxyethanol, propylene glycol methyl ether, n-hexanol, and n-butanol.

In step D2, from the viewpoint of improving efficiency, one or more solvents selected from solvents having a high relative permittivity are preferably used as the reaction solvent, and a mixed solvent using one or more solvents selected from the group consisting of ethanol, propanol, isopropyl alcohol, butanol, isobutanol, sec-butanol, tert-butanol, diglyme, diisopropyl ether, ethyl acetate, tetrahydrofuran, dioxane, methyl ethyl ketone, chloroform, methylene chloride, dichloroethane, benzene, toluene, o-xylene, cyclohexane, hexane, acetonitrile, nitromethane, and pyridine.

In step D2, from the viewpoint of improving the yield, it is preferable to use one or more solvents selected from the group consisting of polar aprotic solvents having a relative permittivity at 20° C. to 30° C. of 20 or more, and one or more solvents selected from the group consisting of ethanol, propanol, isopropyl alcohol, butanol, isobutanol, sec-butanol, tert-butanol, 1,2-dimethoxyethane, diisopropyl ether, ethyl acetate, tetrahydrofuran, dioxane, methyl ethyl ketone, carbon tetrachloride, chloroform, dichloroethane, benzene, toluene, o-xylene, cyclohexane, hexane, acetonitrile, nitromethane, and pyridine, and it is more preferable to use a solvent having a high relative permittivity and a solvent suitable for forming an azeotropic mixture with the solvent having a high relative permittivity in combination.

The amount of the reaction solvent used can be arbitrarily set according to, for example, the substrate, catalyst, and reaction conditions to be used, without particular limitation, and is preferably 0 to 10000 parts by mass, and from the viewpoint of the yield, more preferably 100 to 2000 parts by mass, based on 100 parts by mass of the reaction raw materials.

In step D2, a wide variety of catalysts which function under the reaction conditions for obtaining the compound represented by the formula (0a) or the formula (0b) can be used as the catalyst, and an acid is preferably used as the catalyst. Examples of the acid catalyst include, but are not particularly limited to, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. These acid catalysts can be used singly, or two or more thereof can also be used in combination. Among them, organic acids and solid acids are preferable, and hydrochloric acid or sulfuric acid is more preferably used from the viewpoint of production such as easy availability and handleability.

The amount of the catalyst used can be arbitrarily set according to, for example, the substrate, catalyst, and reaction conditions to be used, without particular limitation, and is preferably 0.0001 to 100 parts by mass, and from the viewpoint of the yield, more preferably 0.001 to 10 parts by mass, based on 100 parts by mass of the reaction raw materials.

In step D2, a polymerization inhibitor may further be used. As the polymerization inhibitor, a wide variety of polymerization inhibitors which function under the dehydration reaction conditions using an acid as the catalyst can be used. In step D2, the polymerization inhibitor is effective but is not an essential component. Preferred examples of polymerization inhibitors include, but are not particularly limited to, hydroquinone, hydroquinone monomethyl ether, 4-tert-butylcatechol, phenothiazine, and an n-oxyl (nitroxide) inhibitor, Prostab® 5415 (bis(1-oxyl-2,2,6,6-tetramethylpiperidin-4-yl)sebacate which is commercially available from Ciba Specialty Chemicals, Tarrytown, NY, CAS #2516-92-9), 4-hydroxy-TEMPO (4-hydroxy-2,2,6,6-tetramethylpiperidin-1-yloxy, which is commercially available from TCI, CAS #2226-96-2), and Uvinul® 4040P (1,6-hexamethylene-bis(N-formyl-N-(1-oxyl-2,2,6,6-tetramethylpiperidin-4-yl)amine) which is commercially available from BASF Corp., Worcester, MA).

The amount of the polymerization inhibitor used can be arbitrarily set according to, for example, the substrate, catalyst, and reaction conditions to be used, without particular limitation, and is preferably 0.0001 to 100 parts by mass, and from the viewpoint of the yield, more preferably 0.001 to 10 parts by mass, based on 100 parts by mass of the reaction raw materials. In addition, a method for bubbling gas components including air and oxygen into a reaction system to increase the effect of the polymerization inhibitor can be arbitrarily used.

In step D2, a polymerization retardant may further be used. As the polymerization retardant, a wide variety of polymerization retardants which function under the dehydration reaction conditions using an acid as the catalyst can be used. In step D2, the polymerization retardant is effective but is not an essential component.

In step D2, a polymerization inhibitor and a polymerization retardant may further be used in combination. The polymerization retardant is well-known in the art as a compound that can delay the polymerization reaction, but cannot prevent all polymerization from occurring. Examples of the polymerization retardant include, but are not particularly limited to, an aromatic nitro compound such as dinitro-ortho-cresol (DNOC) and dinitro butyl phenol (DNBP). The method for producing the polymerization retardant is well-known in the art, and examples thereof include the methods described in U.S. Pat. No. 6,339,177; see Park et al., Polymer (Korea) (1988), 12(8), 710-19), and its use in the control of styrene polymerization is well documented (e.g., see Bushby et al., Polymer (1998), 39(22), 5567-5571).

The amount of the polymerization retardant used can be arbitrarily set according to, for example, the substrate, catalyst, and reaction conditions to be used, without particular limitation, and is preferably 0.0001 to 100 parts by mass, and from the viewpoint of the yield, more preferably 0.001 to 10 parts by mass, based on 100 parts by mass of the reaction raw materials.

As an example of the reaction conditions of step D2 of the present embodiment, the compound represented by the formula (1-1a) or the formula (1-1b), a catalyst, and a solvent are added to a reaction vessel to form a reaction mixture. The reaction vessel is not particularly limited, and a known reaction vessel can be arbitrarily selected. The reaction may be carried out by arbitrarily selecting a publicly known method such as a batch method, a semi-batch method, or a continuous method.

In step D2, the reaction temperature is not particularly limited, and a preferred range thereof may be different according to the concentration of the substrate, the stability of the target substance formed, the selection of the catalyst, and the desired yield. The reaction temperature is preferably 0° C. or more and 200° C. or less, and from the viewpoint of the yield, more preferably 10° C. or more and 190° C. or less, further preferably 25° C. or more and 150° C. or less, and particularly preferably 50° C. or more and 100° C. or less. When 1-(4-hydroxy-3,5-diiodophenyl)ethanol is used as the compound represented by the formula (1-1a) or the formula (1-1b), the reaction temperature is preferably 0° C. or more and 100° C. or less.

In step D2, the reaction pressure is not particularly limited, and a preferred range thereof may be different according to the concentration of the substrate, the stability of the target compound formed, the selection of the catalyst, and the desired yield. The reaction pressure is not particularly limited, and can be adjusted by using an inert gas such as nitrogen, or a suction pump or the like. In the reaction at high pressure, a conventional pressure reaction vessel comprising a shaking vessel, a rocker vessel, a stirred autoclave may be used, without particular limitation. When 1-(4-hydroxy-3,5-diiodophenyl)ethanol is used as the compound represented by the formula (1-1a) or the formula (1-1 b), the reaction pressure is preferably reduced pressure to normal pressure, and more preferably reduced pressure.

In step D2, from the viewpoint of improving the reaction rate, it is preferable to remove water and/or low boiling products such as methanol generated from a dehydration reaction system using an acid as the catalyst, without particular limitation. As the method for removing low boiling products, a conventionally known suitable method can be used. For example, they are preferably removed using evaporation, and are more preferably removed by using evaporation under reduced pressure, without particular limitation. Examples of the method for efficiently removing water and/or low boiling products such as methanol generated from the reaction system include, but are not particularly limited to, a method using a dehydrating material such as a molecular sieve in combination, a method using Dean-Stark, and a method by reduced pressure.

In step D2, the reaction time is not particularly limited, and a preferred range thereof may be different according to the concentration of the substrate, the stability of the target substance formed, the selection of the catalyst, and the desired yield. The reaction time is preferably 6 hours or less, and more preferably 15 minutes or more and 600 minutes or less. When 1-(4-hydroxy-3,5-diiodophenyl)ethanol is used as the compound represented by the formula (1-1a) or the formula (1-1 b), the preferred reaction time is preferably 15 minutes or more and 600 minutes or less.

Step D2 may further comprise a step of isolating and/or purifying the target compound after the dehydration reaction using an acid as the catalyst. Isolation and purification can be conducted by using a conventionally known suitable method after terminating the dehydration reaction using an acid as the catalyst. Examples thereof include, but are not particularly limited to, a method in which the reaction mixture is poured onto ice water and extracted in a solvent such as ethyl acetate or diethyl ether, and the solvent is then removed using evaporation under reduced pressure to recover the product. A high purity target compound may be isolated and purified by a separation and purification method by filtration, concentration, distillation, extraction, crystallization, recrystallization, column chromatography, activated carbon, or the like, which are well-known purification methods in the art, or a combined method thereof.

### <Step PR>

In the present embodiment, step PR is a step of introducing a group represented by the following formula (Y-0). The introduction conditions (such as the solvent, the catalyst, and the reaction temperature) of the group represented by the formula (Y-0) can be arbitrarily selected according to the compound to be introduced and the formula (Y-0).

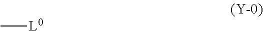

In the formula (Y-0), each L⁰ is independently at least one selected from an alkoxy group, an ester group, an ether group, a thioether group, an acetal group, a thioacetal group, a carboxyalkoxy group, a carbonate ester group, a sulfonyl group, and a silyl group, or a hydrolyzable group.

In the group represented by the formula (Y-0) of the present embodiment, the alkoxy group, the ester group, the ether group, the thioether group, the acetal group, the thioacetal group, the carboxyalkoxy group, the carbonate ester group, the sulfonyl group, and the silyl group, or the hydrolyzable group optionally further have a substituent. Examples of the substituent include, but are not particularly limited to, those mentioned above.

In the group represented by the formula (Y-0) of the present embodiment, the hydrolyzable group is as defined in (Compound A), and is preferably, for example, an alkoxy group, an ester group, an acetal group, a thioacetal group, an oxycarbonyl group, a carboxyalkoxy group, or a carbonate ester group, without particular limitation.

In the present embodiment, the group represented by the formula (Y-0) is further preferably a group represented by the following formula (Y-2).

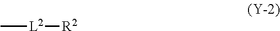

In the formula (Y-2),
each L² is independently at least one divalent linking group selected from the group consisting of an ester group [*¹—(C═O)—O—*²], an acetal group [*¹—O—(C(R²¹)₂)—O—*² (each R²¹ is independently H, or a hydrocarbon group having 1 to 10 carbon atoms)], a carboxyalkoxy group [*¹—O—R²²—(C═O)—O—*² (R²² is a divalent hydrocarbon group having 1 to 10 carbon atoms)], an oxycarbonyl group [*¹—O—(C═O)*²], and a carbonate ester group [*¹—O—(C═O)—O—*²]. In the formula, *1 is a site for binding with A in the formula (1), and *² is a site for binding with R² in the formula (Y-2). R² is as defined in R¹ in the formula (Y-1) and R³ in the formula (Y-3).

In the present embodiment, step PR is preferably a step of introducing the group represented by the formula (Y-0) into a compound represented by the formula (3a0), the formula (3b0), the formula (0a0), the formula (0b0), the formula (1-1a0), or the formula (1-1 b0) to obtain a compound represented by the formula (3a1), the formula (3b1), the formula (0a1), the formula (0b1), the formula (1-1a1), or the formula (1-1 b1), and more preferably a step of obtaining a compound represented by the formula (0b1).

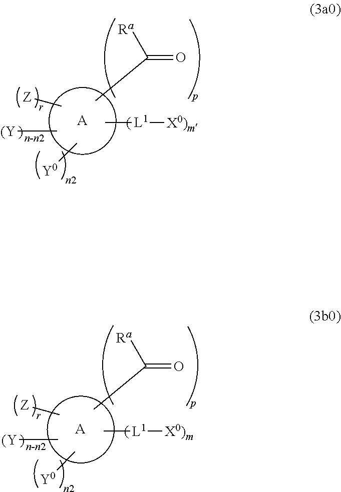

(In the formula (3a0) and the formula (3b0),
X, L¹, Y, R^{a}, Z, A, m, n, p, and r are as defined in the formula (1);
each Y⁰ is independently a hydroxyl group, an amino group, a thiol group, an imide group, a phosphate group, or a halogen, and the amino group, the thiol group, the imide group, or the phosphate group of Y⁰ optionally has a substituent;
X⁰ is H or an organic group having 1 to 30 carbon atoms;
n2 is an integer of 1 or more and n or less; and
m′ is an integer of 0 or more.)

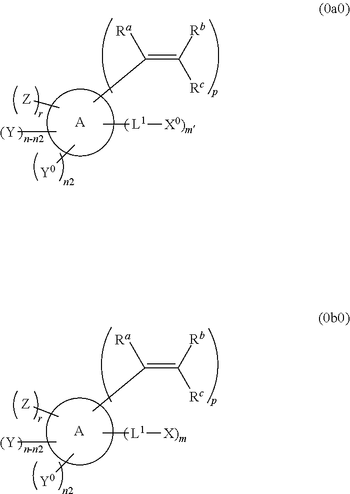

(In the formula (0a0) and the formula (0b0),
X, X⁰, L¹, Y, A, Z, p, m′, n, n2, r, R^{a}, R^{b}, and R^{c} are as defined in the formula (1), the formula (3a), and the formula (3b); and
   Y⁰ is as defined in the formula (3a0).)

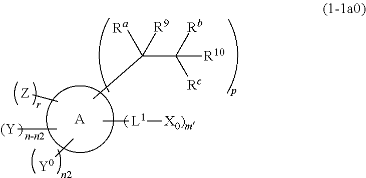

(In the formula (1-1a0),
X⁰, L¹, Y, A, Z, p, m′, n, r, R^{a}, R^{b}, and R^{c} are as defined in the formula (1), the formula (3a), and the formula (3b);
   Y⁰ and n2 are as defined in the formula (3a0); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{c} is an organic group having 1 to 30 carbon atoms and optionally having a substituent.)

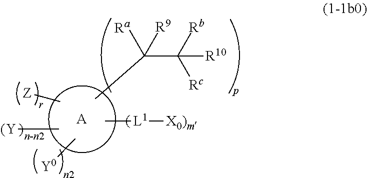

(In the formula (1-1 b0),
X, L¹, Y, A, Z, p, m′, n, r, R^{a}, R^{b}, and R^{c} are as defined in the formula (1), the formula (3a), and the formula (3b);
   Y⁰ and n2 are as defined in the formula (3a0); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{c} is an organic group having 1 to 30 carbon atoms and optionally having a substituent.)

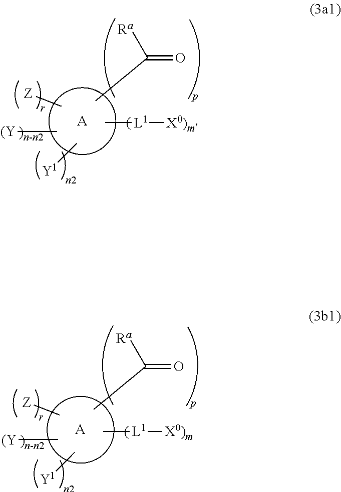

(In the formula (3a1) and the formula (3b1),
X, L¹, Y, R^{a}, Z, A, m, n, p, and r are as defined in the formula (1);
n2 is as defined in the formula (3a0);
Y¹ is a group represented by the formula (Y-0);
X⁰ is H or an organic group having 1 to 30 carbon atoms; and
m′ is an integer of 0 or more.)

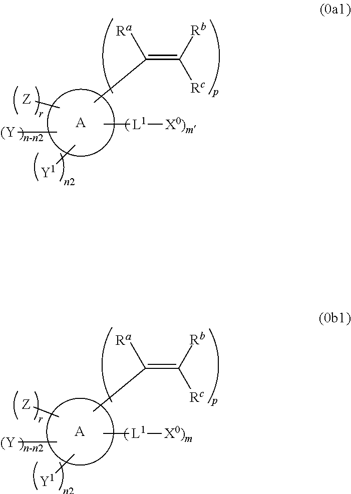

(In the formula (0a1) and the formula (0b1),
X, X⁰, L¹, Y, A, Z, p, m′, n, r, R^{a}, R^{b}, and R^{c} are as defined in the formula (1), the formula (3a), and the formula (3b);
Y¹ is as defined in the formula (3a1); and
n2 is as defined in the formula (3a0).)

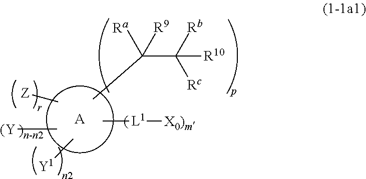

(In the formula (1-1a1),
X⁰, L¹, Y, A, Z, p, m′, n, r, R^{a}, R^{b}, and R^{c} are as defined in the formula (1), the formula (3a), and the formula (3b);
Y¹ is as defined in the formula (3a1);
n2 is as defined in the formula (3a0); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{c} is an organic group having 1 to 30 carbon atoms and optionally having a substituent.)

In the compound represented by the formula (3a1), the formula (3b1), the formula (0a1), the formula (0b1), the formula (1-1a1), or the formula (1-1 b1), Y¹ is preferably a group represented by the formula (Y-3).

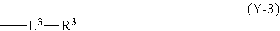

In the formula (Y-3),
L³ is a hydrolyzable group. Examples of the hydrolyzable group include, but are not particularly limited to, at least one divalent linking group selected from the group consisting of an ester group [*¹—(C═O)—O—*²], an acetal group [*¹—O—(C(R²¹)₂)—O—*² (each R²¹ is independently H, or a hydrocarbon group having 1 to 10 carbon atoms)], a carboxyalkoxy group [*¹—O—R²²—(C═O)—O—*² (R²² is a divalent hydrocarbon group having 1 to 10 carbon atoms)], an oxycarbonyl group [*¹—O—(C═O)—*²], and a carbonate ester group [*¹—O—(C═O)—O—*²]. R³ is as defined in R² in the formula (Y-2).

The ester group is preferably a tertiary ester group, from the viewpoint of achieving high sensitivity. In the formula, *1 is a site for binding with A, and *² is a site for binding with R³. Among them, L³ is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of achieving high sensitivity upon use as a copolymer for a resist resin. From the viewpoint of producing a polymer having a stable quality by radical polymerization, an ester group, a carboxyalkoxy group, and a carbonate ester group are preferable.

As another effect, Y¹ is preferably a group represented by the formula (Y-3) to control the polymerization properties of resin and the degree of polymerization in a desired range, when the compound A of the present embodiment is used as a polymerization unit of a copolymer. Since the compound A has a large influence on activity species in the polymer formation reaction due to having an X group and thus the desired control is difficult, variation of copolymer formation derived from the hydrophilic group and polymerization inhibition can be suppressed by having a group represented by the formula (Y-3) in the hydrophilic group in the compound A as a protecting group.

When L³ is a carboxyalkoxy group [*¹—O—R²²—(C═O)—O—*²], R²² may be a divalent hydrocarbon having 1 to 10 carbon atoms, or may have a cyclic structure in which R²² is linked with R² or A in the formula (1).

R³ is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R³ optionally further have a substituent. As the substituent here, the aforementioned substituents are used, but a linear, branched, or cyclic aliphatic group having 1 to 20 carbon atoms or an aromatic group having 6 to 20 carbon atoms are preferable. Among them, R³ is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L³ is preferably *¹—(C═O)—O—*² or a carboxyalkoxy group, because, when L³ is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

When L³ is an oxycarbonyl group, specific examples of the formula (Y-3) include, but are not particularly limited to, the followings.

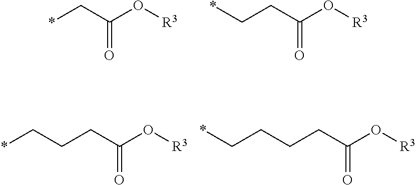

Specific examples of R³ include, but are not particularly limited to, the followings.

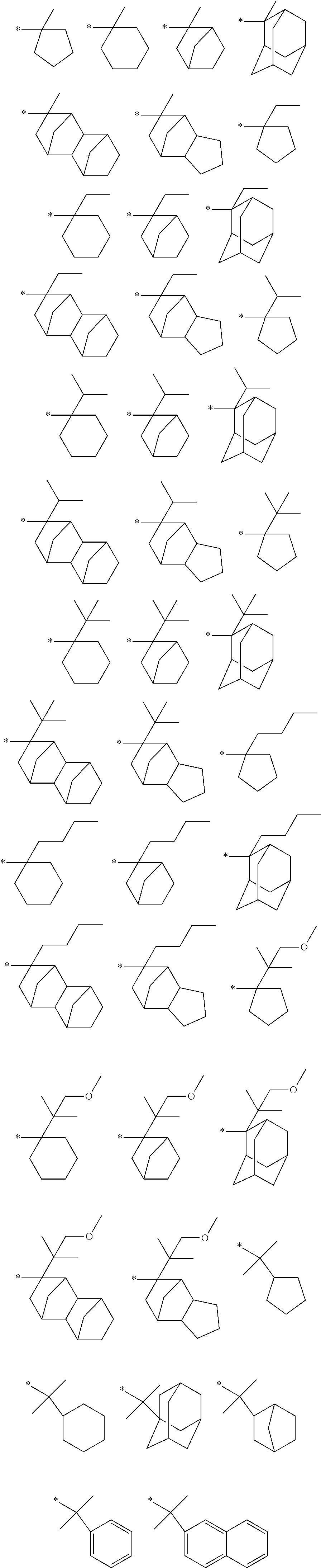

Examples of the group represented by the formula (Y-0), the formula (Y-2), or the formula (Y-3) include, but are not particularly limited to, the followings. Each of them may independently be a group represented by any of the following formulas (Y-1-1) to (Y-1-7).

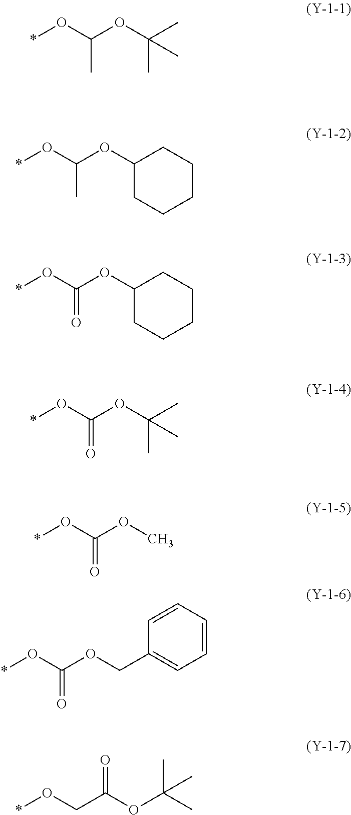

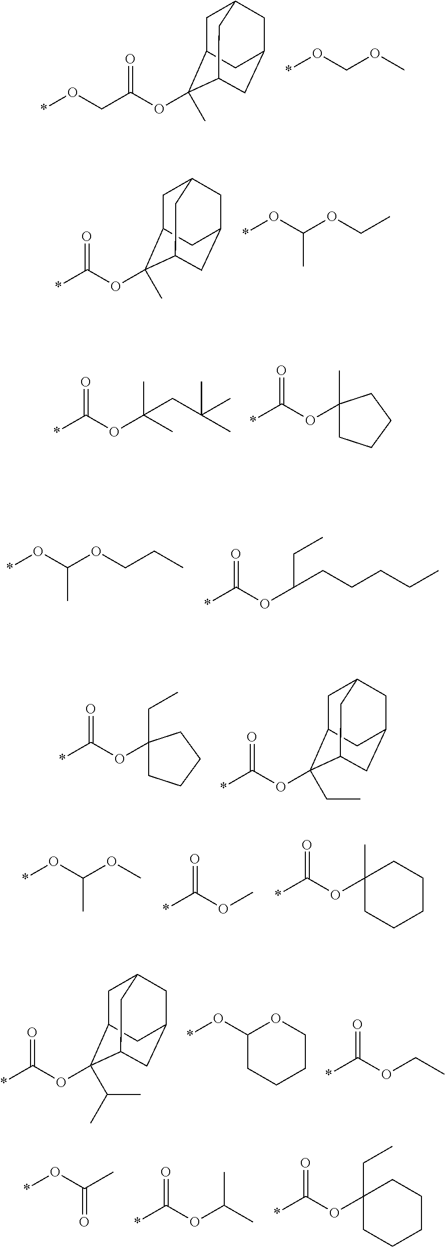

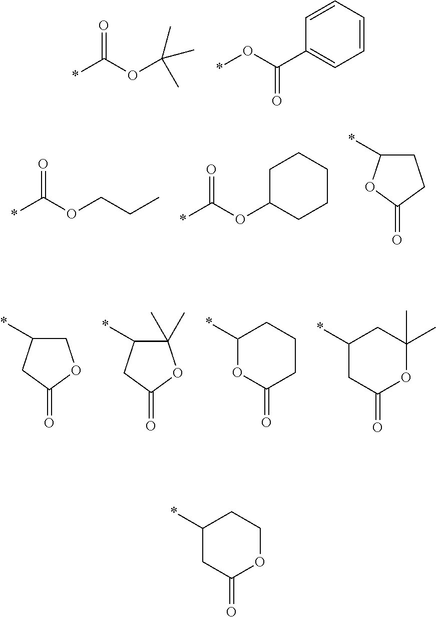

In the group represented by the formula (Y-0), the formula (Y-2), or the formula (Y-3), examples of the alkoxy group include an alkoxy group having 1 or more carbon atoms, and from the viewpoint of the solubility of a resin after the compound is combined with other monomers to form the resin, an alkoxy group having 2 or more carbon atoms is preferable, and an alkoxy group having 3 or more carbon atoms or having a cyclic structure is more preferable.

In the group represented by the formula (Y-0), the formula (Y-2), or the formula (Y-3), specific examples of the alkoxy group include, but are not particularly limited to, the followings.

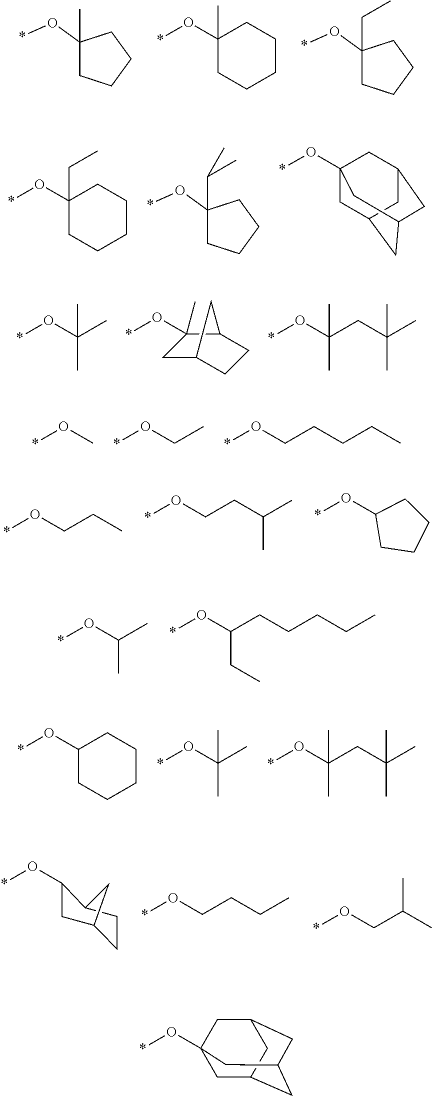

In the group represented by the formula (Y-0), the formula (Y-2), or (Y-3), the amino group or amide group is preferably a primary amino group, a secondary amino group, a tertiary amino group, a group having a quaternary ammonium salt structure, an amide having a substituent, or the like. Examples of the amino group or amide group include, but are not particularly limited to, the followings.

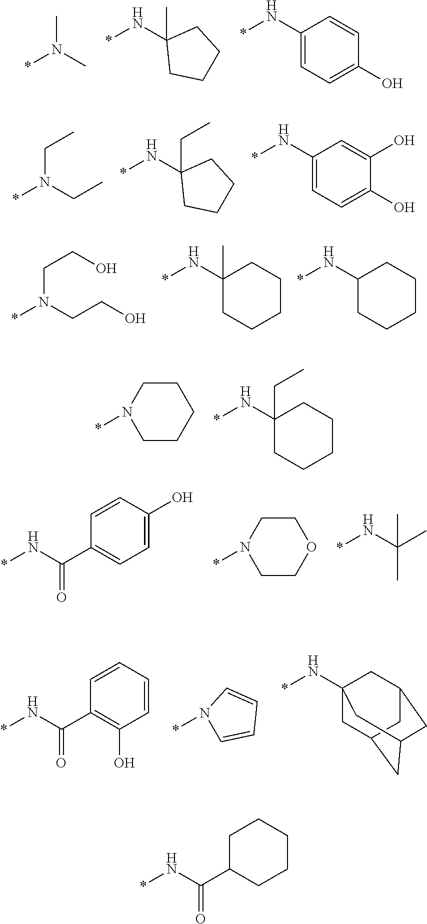

In step PR of the present embodiment, a known method can be used as the method for introducing the group represented by the formula (Y-0), without particular limitation. The method for introducing the group represented by the formula (Y-0) is not particularly limited, and for example, the methods described in Japanese Patent No. 6094969, Japanese Patent Laid-Open No. 2021-050307, Japanese Patent Laid-Open No. 8-157410, Japanese Patent Laid-Open No. 6-192171, Japanese Patent Laid-Open No. 2000-178227, U.S. Pat. No. 5,274,060, Bennech, T, et al. Synthesis 1995, 1. 1, Kumar, H. et al, J. S. Chem. Lett. 1999, 857, and Greene's Protection Groups in Organic Synthesis Fifth Edition (Wiley) can be arbitrarily used.

### (Method for Synthesizing Compound A)

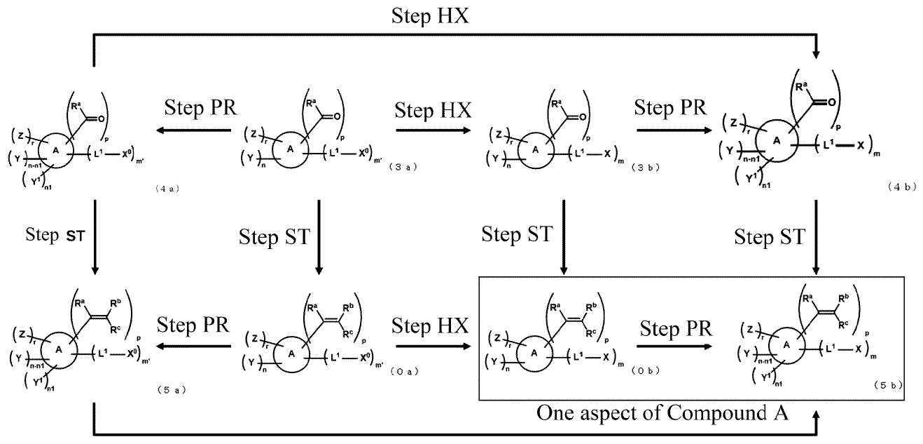

Hereinafter, one example of the method for producing the compound A of the present embodiment will be described with reference to FIG. 1. FIG. 1 is a flow chart of one example of the method for producing the compound A of the present embodiment. Provided that the production method of the present embodiment is not limited to the aspect shown in FIG. 1. In FIG. 1, the compound at the destination of an arrow means that it can be produced from the compound described before the arrow through the step described on the arrow.

In FIG. 1, a compound selected from the compounds represented by the formula (0a), the formula (0b), the formula (3a), the formula (3b), the formula (4a), the formula (4b), or the formula (5a) can be set as the starting compound in the method for producing the compound A of the present embodiment. From the viewpoint of the convenience of providing the starting compound, the compound represented by the formula (3a) or the formula (3b) is preferably set as the starting compound, without particular limitation.

In FIG. 1, the target compound to be produced can be set according to the purpose, and the compound represented by the formula (0b) or the formula (5b) can be set as the target compound in the production method of the present embodiment. That is, the compound represented by the formula (0b) or the formula (5b) is one aspect of the compound A in the present embodiment.

In FIG. 1, when the starting compound and the target compound are set, the target compound can be produced by conducting step HX, step ST, and/or step PR in the order of arrows from the starting compound toward the target compound.

In FIG. 1, for example, when the compound represented by the formula (3a) is set as the starting compound, the compound represented by the formula (3a) can be subjected to step HX, step ST, or step PR, without particular limitation, and is preferably subjected to step HX. When step HX is conducted, the compound represented by the formula (3b) can be obtained from the compound represented by the formula (3a). Then, the compound represented by the formula (3b) can be subjected to step ST or step PR. When step ST is conducted, the compound represented by the formula (0b) can be obtained, and when step PR is conducted, the compound represented by the formula (4b) can be obtained. Then, the compound represented by the formula (4b) can be subjected to step ST to obtain the compound represented by the formula (5b). Alternatively, the compound represented by the formula (0b) can be subjected to step PR to obtain the compound represented by the formula (5b). Provided that, when the compound represented by the formula (0b) is set as the target compound, step PR may not be conducted.

### <Step STPR>

As mentioned above, in one embodiment in the production method of the present embodiment, step ST and step PR can be carried out as a continuous process. Here, the “continuous process” means a method in which, after one step (continuous first step) is carried out, the other step (continuous second step) is continuously carried out without requiring isolation and purification operation of the target substance generated in the continuous first step. Step STPR is a step of carrying out one step of step ST and step PR and then continuously carrying out the other step without carrying out isolation and purification operation. The order for conducting each step of step ST and step PR in step STPR is not limited, and both steps may be carried out in the order from step ST to step PR, or may be carried out in the order from step PR to step ST. Hereinafter, in step STPR that is a continuous process, the step which is previously carried out is referred to as the “continuous first step”, and the step which is carried out later is referred to as the “continuous second step”. As the conditions and materials used in step ST and step PR conducted in step STPR, the same ones as those mentioned above can be arbitrarily employed.

Since step STPR is conducted by the continuous process, a reduction in the yield of the target substance accompanied with the purification operation which is normally carried out after the continuous first step can be avoided, so that a desired derivative can be produced in a high yield. In addition, since step STPR can omit heating operation and concentration operation in isolation and purification operation which is normally carried out after the continuous first step, generation of impurities by the polymerization reaction accompanied with heat or reduced pressure and the like can be suppressed, so that a desired derivative can be stably produced in a high purity. The reason why step STPR can be conducted by the continuous process is not limited; however, in general organic synthesis, impurities are generated in each reaction and the kind of impurity may be different in many cases. Thus, the amount and kind of impurity are accumulated as steps are carried out, and if purification is carried out only one time at the end, the load is large and purification is made difficult. Accordingly, in a general organic synthesis reaction, isolation and purification operation is required after each step. On the other hand, almost no impurities are generated in step PR in step STPR, the phosphine oxide generated in step ST is only required to be the target to be purified. In addition, the method for removing phosphine oxide has a high versatility for a substrate having a double bond, so that the same method can also be applied to the substrate after being derivatized by step PR. Further, since the reaction conditions required in step PR are not very severe, it is easy to use the same solvent and base as those in step ST. For these reasons, step STPR can be conducted by the continuous process.

In step STPR, the product generated in the continuous first step may remain in the mother liquid. The product of the continuous first step may remain in a form where the reactant in the continuous first step acts (e.g., a form where an alcohol acts with a base to form an alkoxide) as it is.

In step STPR, the continuous first step and the continuous second step may be carried out in the same reaction vessel, or may be carried out in different reaction vessels. That is, in step STPR, step ST and step PR can be carried out in the same reaction vessel. The method for carrying out the continuous first step and the continuous second step in the same reaction vessel is not particularly limited, and for example, there are a method for directly putting the reactant of the continuous second step into the reaction solution of the continuous first step, and a method in which a substrate is put into a reaction vessel charged with the reactant used in the continuous first step and the continuous second step, and the reactions in the first step and the second step are continuously carried out.

In step STPR, the method for carrying out the continuous first step and the continuous second step in different reaction vessels is not particularly limited, and for example, there is a method for directly putting the reaction solution of the continuous first step which is subjected to operation such as concentration if required, into a reaction vessel in which the reactant of the continuous second step and the reaction solvent are provided. Whether the continuous first step and the continuous second step are carried out in the same reaction vessel or in different reaction vessels is selected according to the reaction form to be conducted, the desired yield, and the desired purity. From the viewpoint of suppressing the contamination of impurities accompanied with the transfer between reaction vessels, making the operation convenience, and efficiently producing the target substance, the continuous first step and the continuous second step are preferably carried out in the same reaction vessel.

Since step STPR employs the continuous process, new charging of a reactant which is normally required in the continuous second step according to the reaction form can be omitted. For example, when a base is required in the reaction forms of both the continuous first step and the continuous second step, the base used in the continuous first step can be continuously used in the continuous second step. When the action of the base continues also in the continuous second step, charging of a new base can be omitted in the continuous second step. For example, in step STPR, after one step is carried out, the other step can be continuously carried out without exchanging the solvent.

In the present embodiment, when an organic phosphorus compound is used and the obtained reaction solution contains phosphine oxide in step STPR in which step ST and step PR are carried out as a continuous process, step STPR may further comprise a step of removing phosphine oxide. In step STPR, for example, the aforementioned step (W) can be employed as step ST, and a method used in the aforementioned step W2 can be used as a method for removing phosphine oxide without particular limitation.

For example, to utilize the reaction of forming an unsaturated double bond from the carbonyl moiety of the carbonyl compound B (such as a Wittig reaction) in step (W) in step STPR as in the aforementioned step W2, an organic phosphorus compound can be used in step W2. Further, a method in which phosphine oxide generated by the Wittig reaction or the like is solidified and the solidified phosphine oxide is removed by solid-liquid separation can be employed in step STPR. Examples of the method in which phosphine oxide generated is solidified by the Wittig reaction or the like and the solidified phosphine oxide is removed by solid-liquid separation include a method in which an acid is further added to solidify phosphine oxide as a composite with the acid and the solidified phosphine oxide is removed by solid-liquid separation, and a method in which phosphine oxide is solidified by changing the solvent system and the solidified phosphine oxide is removed by solid-liquid separation. In step STPR, the timing for adding an acid to solidify phosphine oxide as a composite with the acid and removing the solidified phosphine oxide by solid-liquid separation is not particularly limited. In both cases where step STPR is carried out in the order from step PR to step ST and in the order from step ST to step PR, the above solidification and removal are preferably conducted after the action of the base in the system is weakened, specifically, after the system is neutralized in the continuous second step.

Also in step STPR, the amounts of substances of acid and phosphine oxide preferably satisfy the following expression 1 as mentioned above.

$$0.5 \leq \frac{\sum ( Amount of substance of the acid ) \times ( Valence of the acid )}{( Amount of substance of the phosphine oxide )} \leq 5$$

### <Impurity Removal Step>

The production method of the present embodiment may comprise an impurity removal step of removing impurities using filtration using a filter or an adsorbent, in addition to the step of removing phosphine oxide in the aforementioned step W2. For the removal of impurities, methods such as separation treatment and crystallization are generally selected. On the other hand, to achieve the object of producing the target compound in a high purity by the production method of the present embodiment, it is effective to achieve a high purity by a further purification method, because, among impurities produced as by-products during production, by-products of oligomer size such as a dimer and a trimer and impurities having a high molecular weight have a compound structure and dissolution properties similar to those of the target substance and, on the other hand, performance influence on applications are concerned. The further purification method is not particularly limited, and an impurity removal step using an adsorbent and an impurity removal step using reslurry are preferably used. A plurality of kinds of filtration using a filter and adsorbents may be arbitrarily combined and used.

Examples of the adsorbent include known adsorbents such as inorganic adsorbents such as alumina, activated alumina, silica gel, modified silica gel, silica-alumina, zeolite (such as synthetic zeolite), hydrotalcite, florisil, activated clay, diatomaceous earth, and mica; and organic adsorbents such as activated charcoal, a molecular sieve, and an ion exchange resin.

In the removal using the adsorbent, when a clean solid surface is brought into contact with a target component, the target component is adsorbed on the surface by the interaction between the atoms on the solid surface and the target component. By utilizing this adsorption action, a specific component can be adsorbed and removed. Adsorption phenomenon can be classified into two types, physisorption and chemisorption, according to the way of interaction.

Among them, “physisorption” refers to, among phenomenons in which atoms and molecules are adsorbed on the solid surface, adsorption mainly caused by the van der Waals interaction between gas molecules and surface atoms. Physisorption is characterized by low specificity due to substances, a high adsorption rate, low heat of adsorption (to 20 kJmol⁻¹), the occurrence of multilayer adsorption, reversible elimination, and no dissociation.

On the other hand, “chemisorption” refers to, among phenomenons in which atoms and molecules are adsorbed on the solid surface, adsorption mainly caused by chemical bond generation and charge transfer interaction. There are many gas molecules and gas atoms that are chemically adsorbed on metals and metal oxides. Chemisorption is characterized by strong specificity due to substances, a low adsorption rate, high heat of adsorption (some hundreds kJ mol⁻¹), and the occurrence of only single layer adsorption.

As the purification step, a reslurry treatment can be selected. In the reslurry treatment, purification is carried out by a method in which a target substance in a solid state is subjected to a distribution treatment in a solvent, and impurities are dissolved in a reslurry solvent and removed while maintaining the target substance in a solid state. The reslurry treatment can be carried out by arbitrarily selecting the kind of solvent, the temperature, and a dispersion means such as stirring or sonication.

A reslurry solvent having a low solubility of the target substance is preferable and can be arbitrarily selected from solvents having a solubility of 5% or less, more preferably 3% or less, further preferably 1% or less, and still more preferably 0.1% or less. By combining an arbitrary solvent as an auxiliary solvent, the reslurry solvent can be adjusted so that the target substance can be recovered while ensuring the solubility of impurities to be removed.

Examples of the impurities to be removed include residues of reaction reagents, reaction products of dimers and raw materials with solvents and reaction reagents as reaction by-products or side reaction products, modified products of reaction products, and oligomers or polymers derived from raw materials and reaction products. There is concern that containing these impurities may affect items such as the productivity, resolution, roughness, defects, and yield in semiconductor lithography, particularly when the compound obtained by the present embodiment is applied to a material for lithography. Among them, the reslurry treatment particularly has an effect of removing side reaction products and oligomers or polymers derived from raw materials and reaction products.

The timing for carrying out the impurity removal step is not particularly limited, and can be, for example, arbitrarily conducted in step C, step D, or step PR in step HX or step ST as a pretreatment of charging a reaction raw material or a reaction reagent in the main reaction, or as a step added to a posttreatment step after the reaction.

### <Crystallization Step>

The production method of the present embodiment can comprise a crystallization step of obtaining a compound as the target substance by industrial crystallization. Industrial crystallization refers to a method for forming a supersaturated state by any method, and then forming a new equilibrium phase containing a solid based on the supersaturation degree, that is, the extent of deviation from the equilibrium state. Thus, by conducting the crystallization step, nucleation and crystal growth can be promoted by the supersaturation degree, and a specific component can be solidified in a high purity and separated. As the method for forming a supersaturated state, a method including temperature control, a method for changing the formulation of a solvent, a method for adding a precipitant, or the like can be arbitrarily selected.

As the kind of industrial crystallization, a method in which supersaturation is formed by cooling the raw material solution by cooling crystallization to reduce the solubility, a method in which supersaturation is generated by heat-concentrating the raw material solution by evaporation crystallization, a method in which supersaturation is formed by adding a solvent hardly soluble to a solute by anti-solvent crystallization to reduce the solubility, a method in which a supersaturated state is formed by proceeding the chemical reaction by addition of a precipitant or blowing of reaction gas as reactive crystallization, or the like can be arbitrarily selected.

From the viewpoint of yield and purity and from the viewpoint of efficiently carrying out crystallization while avoiding problems such as scaling due to inclusion of a solvent, the temperature dependence of the solubility of the target component and the width of the conditions for forming a supersaturated state are clarified with respect to the temperature and the concentration, then a solution containing the target component in a metastable zone between a solubility curve and a supersolubility curve is formed, crystal nuclei are formed, thereby promoting moderate crystal growth. Thus, crystallization that gives a crystalline state allowing easy recovering operation can be carried out.

As a specific method of scale-up, methods described in “Optimization of Separation Process and Procedure of Scale-up” (TECHNICAL INFORMATION INSTITUTE CO., LTD) Chapter 4, “Correct Procedure of Scale-up of Production Process and Trouble Countermeasure Case Studies” (TECHNICAL INFORMATION INSTITUTE CO., LTD) Chapter 4, Section 1, and the like can be arbitrarily used.

In the production method of the present embodiment, the purity of the target substance can be improved by a drying step. In the drying step, by arbitrarily combining a separation step, a concentration step, and a crystallization step with a crude of a target substance synthesized in a synthesis step, a target substance or crude of the target substance having an improved purity can be obtained. The purity of the obtained crude can be improved by volatilizing and removing impurity components including remaining solvent components by drying. As the drying method, a step of removing a volatile component by utilizing a difference in vapor pressure is exhibited. To improve purification efficiency, a method for volatilizing remaining components by adding thermal energy, a method for increasing the volume of the gas phase component in contact with the crude by utilizing fluid flow, a method for reducing atmospheric pressure, a freeze-drying method, or the like can be arbitrarily used.

As the method for drying the compound of the present embodiment, a means for suppressing modification of the target compound during drying is preferably used in combination. Examples of the cause of modification include an oxidation reaction due to an oxidizing component such as an oxygen-containing component in the atmosphere, a polymerization reaction which is considered to be due to a thermal radical generated by thermal energy on the unsaturated double bond moiety, and elimination of the halogen moiety by thermal energy. Examples of means for suppressing these include suppression of radical-derived modification by blowing of oxygen in the drying step, and combination use of an antioxidative component.

As an antioxidant, known materials can be arbitrarily used. Examples of the antioxidant include p-benzoquinone, naphthoquinone, phenanthraquinone, toluquinone, 2,6-diphenyl-p-benzoquinone, 2,5-diacetoxy-p-benzoquinone, 2,5-dicaproxy-p-benzoquinone, 2,5-diacyloxy-p-benzoquinone, hydroquinone, p-t-butylcatechol, 2,5-di-t-butylhydroquinone, mono-t-butylhydroquinone, 2,5-di-t-amylhydroquinone, di-t-butyl paracresol hydroquinone monomethyl ether, anaphthol, methoquinone, BHT, TEMPO, and OH-TEMPO. From the viewpoint of versatility, methoquinone, BHT, TEMPO, or OH-TEMPO is preferably used.

As the application method of the antioxidant, a method for using an appropriate amount of antioxidant in a solution containing the target compound in combination in a step before drying (the crystallization step or the concentration step), a method for treating a rinsing solution containing an antioxidant component in a rinsing treatment after filtration in the crystallization step, or the like can be arbitrarily used.

### [Polymer]

The polymer of the present embodiment contains a constitutional unit corresponding to the aforementioned compound A. By containing the constitutional unit corresponding to the compound A, the polymer in the present embodiment can increase the sensitivity to an exposure light source when being blended in a resist composition. In particular, the polymer can exhibit sufficient sensitivity and can form good thin line patterns having a narrow line width and requiring a higher exposure sensitivity to a resist, even in the case of using an extreme ultraviolet ray as the exposure light source. The polymer in the present embodiment may contain a constitutional unit derived from a further monomer other than the constitutional unit corresponding to the compound A. As the further monomer to be copolymerized with the compound A, it is preferable to contain a polymerization unit that has an aromatic compound having an unsaturated double bond as a substituent, as a polymerization unit, and has a functional group for improving solubility in an alkaline developing solution by the action of an acid or a base.

Examples of other monomers include, but are not particularly limited to, those described in International Publication No. WO 2016/125782, International Publication No. WO 2015/115613, Japanese Patent Laid-Open No. 2015/117305, International Publication No. WO 2014/175275, and Japanese Patent Laid-Open No. 2012/162498, or a compound represented by the following formula (C1) or formula (C2). Among them, the compound represented by the following formula (C1) or formula (C2) is preferable.

The method for producing the polymer in the present embodiment will be described. The polymerization reaction is carried out by dissolving a monomer as a constitutional unit in a solvent and adding a polymerization initiator while heating or cooling. The reaction conditions can be arbitrarily set depending on the kind of polymerization initiator, the initiation method such as heat or light, the temperature, the pressure, the concentration, the solvent, the additive, and the like. Examples of the polymerization initiator include a radical polymerization initiator such as azoisobutyronitrile and peroxide, and an anion polymerization initiator such as alkyllithium and a Grignard reagent.

As the solvent used in the polymerization reaction, a commonly available product can be used. For example, a wide variety of solvents such as alcohols, ethers, hydrocarbons, and halogenated solvents can be arbitrarily used within a range not inhibiting the reaction. Within the above range not inhibiting the reaction, a plurality of solvents can be used as a mixture.

The polymer in the present embodiment obtained in the polymerization reaction can be purified by a publicly known method. Specifically, ultrafiltration, crystallization, microfiltration, acid washing, water washing at an electrical conductivity of 10 mS/m or less, and extraction can be used in combination.

### [Composition]

The composition of the present embodiment at least contains at least one of the compound A and the polymer having a constitutional unit corresponding to the compound A. The composition of the present embodiment may contain various additives and the like according to the use application of the compound A and the like. Examples of the composition of the present embodiment include a composition for film formation.

### [Composition for Film Formation]

The composition for film formation of the present embodiment at least contains at least one of the compound A and the polymer having a constitutional unit corresponding to the compound A. The composition for film formation of the present embodiment can be used for, without particular limitation, film formation purposes for lithography, for example, resist film formation purposes (that is, a “resist composition”), and furthermore, upper layer film formation purposes (that is, a “composition for upper layer film formation”), intermediate layer formation purposes (that is, a “composition for intermediate layer formation”), underlayer film formation purposes (that is, a “composition for underlayer film formation”), and the like. According to the composition of the present embodiment, not only a film having high sensitivity can be formed, but also a good resist pattern shape can be imparted.

The composition for film formation of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

The composition for film formation of the present embodiment may further contain, for example, an acid generating agent, a base generating agent, or a base compound. The composition for film formation of the present embodiment contains the compound A or the polymer in the present embodiment, and may contain other components such as a base material, a solvent, or an acid diffusion controlling agent, if required. Hereinafter, each of these components will be described.

### [Base Material]

The “base material” in the present embodiment is the compound A or a compound other than the polymer in the present embodiment and means a base material applied as a resist for g-ray, i-ray, KrF excimer laser (248 nm), ArF excimer laser (193 nm), extreme ultraviolet (EUV) lithography (13.5 nm) or electron beam (EB) (for example, a base material for lithography or a base material for resist). These base materials can be used as the base material in the present embodiment without particular limitation. Examples of the base material include a phenol novolac resin, a cresol novolac resin, a hydroxystyrene resin, a (meth)acrylic resin, and a hydroxystyrene-(meth)acrylic copolymer, a cycloolefin-maleic anhydride copolymer, a cycloolefin, a vinyl ether-maleic anhydride copolymer, and an inorganic resist material having a metallic element such as titanium, tin, hafnium, and zirconium, and derivatives thereof. Among them, from the viewpoint of the shape of a resist pattern to be obtained, preferable are a phenol novolac resin, a cresol novolac resin, a hydroxystyrene resin, a (meth)acrylic resin, a hydroxystyrene-(meth)acrylic copolymer, and an inorganic resist material having a metallic element such as titanium, tin, hafnium, and zirconium, and derivatives thereof.

Examples of the derivative include, but are not particularly limited to, those to which a dissociation group is introduced and those to which a crosslinkable group is introduced. The above derivative to which a dissociation group or a crosslinkable group is introduced can exhibit dissociation reaction or crosslinking reaction through the effect of light, acid or the like.

The “dissociation group” refers to a characteristic group that is cleaved to generate a functional group that alters solubility, such as an alkali soluble group. Examples of the alkali soluble group include, but are not particularly limited to, a phenolic hydroxy group, a carboxyl group, a sulfonic acid group, and a hexafluoroisopropanol group, and a phenolic hydroxy group and a carboxyl group are preferable, and a phenolic hydroxy group is particularly preferable.

The “crosslinking group” refers to a group that crosslinks in the presence of a catalyst or without a catalyst. Examples of the crosslinking group include, but are not particularly limited to, an alkoxy group having 1 to 20 carbon atoms, a group having an allyl group, a group having a (meth)acryloyl group, a group having an epoxy (meth)acryloyl group, a group having a hydroxy group, a group having a urethane (meth)acryloyl group, a group having a glycidyl group, and a group having a vinyl-containing phenylmethyl group.

### [Solvent]

As the solvent in the present embodiment, a known solvent can be arbitrarily used as long as it can at least dissolve the compound A or the polymer in the present embodiment mentioned above. Examples of the solvent include, but are not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate (PGMEA), propylene glycol monoethyl ether acetate, propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; lactate esters such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as acetone, 2-butanone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; and lactones such as γ-lactone. The solvent used in the present embodiment is preferably a safe solvent, more preferably at least one selected from PGMEA, PGME, CHN, CPN, 2-heptanone, anisole, butyl acetate and ethyl lactate, and further preferably at least one selected from PGMEA, PGME, CHN, CPN and ethyl lactate.

In the composition for film formation of the present embodiment, the solid component concentration is not particularly limited, but is preferably 1 to 80% by mass, more preferably 1 to 50% by mass, further preferably 1 to 30% by mass, and still more preferably 1 to 10% by mass, based on the total mass of the composition for film formation.

### [Acid Generating Agent]

It is preferable that the composition for film formation of the present embodiment contain one or more acid generating agents that directly or indirectly generate an acid by radiation irradiation. Radiation is at least one selected from the group consisting of visible light, ultraviolet, excimer laser, electron beam, extreme ultraviolet (EUV), X-ray, and ion beam. The acid generating agent is not particularly limited, and, for example, an acid generating agent described in International Publication No. WO 2013/024778 can be used. The acid generating agent can be used alone or in combination of two or more kinds.

The amount of the acid generating agent blended is preferably 0.001 to 49% by mass, more preferably 1 to 40% by mass, further preferably 3 to 30% by mass, and still more preferably 5 to 25% by mass, based on the total mass of the solid components. By using the acid generating agent within the above range, a pattern profile with high sensitivity and low edge roughness tends to be obtained. In the present embodiment, the acid generation method is not particularly limited, as long as an acid is generated in the system. By using excimer laser instead of ultraviolet such as g-ray and i-ray, finer processing is possible, and also by using electron beam, extreme ultraviolet, X-ray, or ion beam as a high energy ray, further finer processing is possible.

### [Acid Diffusion Controlling Agent]

The composition for film formation of the present embodiment may contain the acid diffusion controlling agent. The acid diffusion controlling agent controls diffusion of an acid generated from an acid generating agent by radiation irradiation in a resist film to inhibit any unpreferable chemical reaction in an unexposed region or the like. By using the acid diffusion controlling agent, there is a tendency that the storage stability of the composition of the present embodiment can be improved. Also, by using the acid diffusion controlling agent, there is a tendency that not only the resolution of a film formed by using the composition of the present embodiment can be improved, but also the line width change of a resist pattern due to variation in the post exposure delay time before radiation irradiation and the post exposure delay time after radiation irradiation can also be suppressed, making the composition excellent in process stability. Examples of the acid diffusion controlling agent include, but are not particularly limited to, a radiation degradable basic compound such as a nitrogen atom containing basic compound, a basic sulfonium compound, and a basic iodonium compound.

The acid diffusion controlling agent is not particularly limited, and, for example, an acid diffusion controlling agent described in International Publication No. WO 2013/024778 can be used. The acid diffusion controlling agent can be used alone or in combination of two or more kinds.

The amount of the acid diffusion controlling agent blended is preferably 0.001 to 49% by mass, more preferably 0.01 to 10% by mass, further preferably 0.01 to 5% by mass, and further preferably 0.01 to 3% by mass, based on the total mass of the solid components. When the amount of the acid diffusion controlling agent blended is within the above range, there is a tendency that a decrease in resolution, and deterioration of the pattern shape and the dimension fidelity or the like can be prevented. Moreover, even though the post exposure delay time from electron beam irradiation to heating after radiation irradiation becomes longer, deterioration of the shape of the pattern upper layer portion can be suppressed. When the amount of the acid diffusion controlling agent blended is 10% by mass or less, there is a tendency that a decrease in sensitivity, and developability of the unexposed portion or the like can be prevented. Also, by using such an acid diffusion controlling agent, there is a tendency that the storage stability of a resist composition is improved, also along with improvement of the resolution, the line width change of a resist pattern due to variation in the post exposure delay time before radiation irradiation and the post exposure delay time after radiation irradiation can be suppressed, making the composition excellent in process stability.

### [Base Generating Agent]

The case where the base generating agent is a photobase generating agent will be described.

The photobase generating agent generates a base upon exposure and does not exhibit activity under normal conditions at normal temperature and pressure, but is not particularly limited as long as the photobase generating agent generates a base (basic substance) upon irradiation with an electromagnetic wave and heating as an external stimulus.

The photobase generating agent which can be used in the present embodiment is not particularly limited, and a publicly known one can be used, and examples thereof include, for example, a carbamate derivative, an amide derivative, an imide derivative, an α-cobalt complex, an imidazole derivative, a cinnamic acid amide derivative, and an oxime derivative.

The basic substance generated from the photobase generating agent is not particularly limited, and examples thereof include compounds having an amino group, particularly monoamines, polyamines such as diamines, and amidines. The basic substance to be generated is preferably a compound having an amino group with a higher basicity (a higher pKa value of the conjugate acid) from the viewpoint of sensitivity and resolution.

Examples of the photobase generating agent include, for example, base generating agents having a cinnamic acid amide structure as disclosed in Japanese Patent Laid-Open No. 2009/80452 and International Publication NO. WO 2009/123122; base generating agents having a carbamate structure as disclosed in Japanese Patent Laid-Open No. 2006/189591 and Japanese Patent Laid-Open No. 2008/247747; base generating agents having an oxime structure or a carbamoyloxime structure as disclosed in Japanese Patent Laid-Open No. 2007/249013 and Japanese Patent Laid-Open No. 2008/003581; and compounds described in Japanese Patent Laid-Open No. 2010/243773, but these are not limited thereto, and other known structures of base generating agents can be used.

The photobase generating agent can be used alone or in combination of two or more kinds.

The preferred content of the photobase generating agent in actinic ray or radiation sensitive resin composition is similar to the preferred content of the aforementioned photoacid generating agent in actinic ray or radiation sensitive resin composition.

### [Base Compound]

The base compound is not particularly limited, and a base compound described in International Publication No. WO 2013/024778 can be used. The base compound can be used as one kind or in combination of two or more kinds.

The amount of the base compound blended is preferably 0.001 to 49% by mass, more preferably 0.01 to 5% by mass, and further preferably 0.01 to 3% by mass, based on the total mass of the solid components.

### [Additional Component]

To the composition for film formation of the present embodiment, if required, as the other component, one kind or two or more kinds of various additives such as a cross-linking agent, a dissolution promoting agent, a dissolution controlling agent, a sensitizing agent, a surfactant, and an organic carboxylic acid or an oxo acid of phosphorus or derivative thereof can be added.

### (Cross-Linking Agent)

The composition for film formation of the present embodiment may contain the cross-linking agent. The cross-linking agent may crosslink at least one of the compound A, the polymer in the present embodiment, and the base material. It is preferable that the crosslinking agent be an acid crosslinking agent capable of intramolecularly or intermolecularly crosslinking the base material in the presence of the acid generated from the acid generating agent. Examples of such an acid crosslinking agent can include a compound having one or more groups capable of crosslinking the base material (hereinafter, referred to as a “crosslinkable group”).

Examples of the crosslinkable group include: (i) a hydroxyalkyl group or a group derived therefrom, such as a hydroxy group, a hydroxyalkyl group (alkyl group having 1 to 6 carbon atoms), alkoxy having 1 to 6 carbon atoms (alkyl group having 1 to 6 carbon atoms) and acetoxy (alkyl group having 1 to 6 carbon atoms); (ii) a carbonyl group or a group derived therefrom, such as a formyl group and a carboxy (alkyl group having 1 to 6 carbon atoms); (iii) a nitrogenous group containing group such as a dimethylaminomethyl group, a diethylaminomethyl group, a dimethylolaminomethyl group, a diethylolaminomethyl group and a morpholinomethyl group; (iv) a glycidyl group containing group such as a glycidyl ether group, a glycidyl ester group and a glycidylamino group; (v) a group derived from an aromatic group such as an allyloxy having 1 to 6 carbon atoms (alkyl group having 1 to 6 carbon atoms) and an aralkyloxy having 1 to 6 carbon atoms (alkyl group having 1 to 6 carbon atoms) such as a benzyloxymethyl group and a benzoyloxymethyl group; and (vi) a polymerizable multiple bond containing group such as a vinyl group and an isopropenyl group. As the crosslinkable group of the crosslinking agent in the present embodiment, a hydroxyalkyl group, an alkoxyalkyl group and the like are preferable, and an alkoxymethyl group is particularly preferable.

The crosslinking agent having the crosslinkable group is not particularly limited, and, for example, an acid crosslinking agent described in International Publication No. WO 2013/024778 can be used. The crosslinking agent can be used alone or in combination of two or more kinds.

The amount of the cross-linking agent blended in the present embodiment is preferably 50% by mass or less, more preferably 40% by mass or less, further preferably 30% by mass or less, and further preferably 20% by mass or less, based on the total mass of the solid components.

### (Dissolution Promoting Agent)

The dissolution promoting agent is a component having a function of, when the solubility of a solid component in a developing solution is too low, increasing the solubility of the solid component in a developing solution to moderately increase the dissolution rate of the compound upon developing. As the dissolution promoting agent, those having a low molecular weight are preferable, and examples thereof include a phenolic compound having a low molecular weight. Examples of the phenolic compound having a low molecular weight include a bisphenol and a tris(hydroxyphenyl)methane. These dissolution promoting agents can be used alone or in mixture of two or more kinds.

The amount of the dissolution promoting agent blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Dissolution Controlling Agent)

The dissolution controlling agent is a component having a function of, when the solubility of a solid component in a developing solution is too high, controlling the solubility of the solid component in a developing solution to moderately decrease the dissolution rate upon developing. As such a dissolution controlling agent, the one which does not chemically change in steps such as calcination of resist coating, radiation irradiation, and development is preferable.

The dissolution controlling agent is not particularly limited, and examples thereof include an aromatic hydrocarbon such as phenanthrene, anthracene and acenaphthene; a ketone such as acetophenone, benzophenone and phenyl naphthyl ketone; and a sulfone such as methyl phenyl sulfone, diphenyl sulfone and dinaphthyl sulfone. These dissolution controlling agents can be used alone or in combination of two or more kinds.

The amount of the dissolution controlling agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Sensitizing Agent)

The sensitizing agent is a component having a function of absorbing irradiated radiation energy, transmitting the energy to the acid generating agent, and thereby increasing the amount of acid generated, and improving the apparent sensitivity of a resist. Examples of such a sensitizing agent can include, but are not particularly limited to, a benzophenone, a biacetyl, a pyrene, a phenothiazine and a fluorene. These sensitizing agents can be used alone or in combination of two or more kinds.

The amount of the sensitizing agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Surfactant)

The surfactant is a component having a function of improving coatability and striation of the composition of the present embodiment, and developability of a resist or the like. The surfactant may be any of anionic, cationic, nonionic, and amphoteric surfactants. Preferable examples of the surfactant include a nonionic surfactant. The nonionic surfactant has a good affinity with a solvent to be used in production of the composition of the present embodiment, and can further enhance the effects of the composition of the present embodiment. Examples of the nonionic surfactant include, but are not particularly limited to, a polyoxyethylene higher alkyl ether, a polyoxyethylene higher alkyl phenyl ether, and a higher fatty acid diester of polyethylene glycol. Examples of commercially available products of these surfactants include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.).

The amount of the surfactant blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Organic Carboxylic Acid or Oxo Acid of Phosphorus or Derivative Thereof)

For the purpose of prevention of sensitivity deterioration or improvement of a resist pattern shape and post exposure delay stability or the like, and as an additional optional component, the composition of the present embodiment can contain an organic carboxylic acid or an oxo acid of phosphorus or derivative thereof. The organic carboxylic acid or the oxo acid of phosphorus or derivative thereof can be used in combination with the acid diffusion controlling agent, or may be used alone. Examples of suitable organic carboxylic acids include malonic acid, citric acid, malic acid, succinic acid, benzoic acid and salicylic acid. Examples of the oxo acid of phosphorus or derivative thereof include phosphoric acid or derivative thereof such as ester including phosphoric acid, di-n-butyl phosphate and diphenyl phosphate; phosphonic acid or derivative thereof such as ester including phosphonic acid, dimethyl phosphonate, di-n-butyl phosphonate, phenylphosphonic acid, diphenyl phosphonate and dibenzyl phosphonate; and phosphinic acid and derivative thereof such as ester including phosphinic acid and phenylphosphinic acid. Among them, phosphonic acid is particularly preferable.

The organic carboxylic acid or the oxo acid of phosphorus or derivative thereof can be used alone or in combination of two or more kinds. The amount of the organic carboxylic acid or the oxo acid of phosphorus or derivative thereof blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Additional Additive Agent)

Furthermore, the composition of the present embodiment can contain one kind or two kinds or more of additives other than the components mentioned above, if required. Examples of such an additive include a dye, a pigment and an adhesion aid. For example, when the composition is blended with a dye or a pigment, a latent image of the exposed portion is visualized and influence of halation upon exposure can be alleviated, which is preferable. Also, when the composition is blended with an adhesion aid, adhesiveness to a substrate can be improved, which is preferable. Furthermore, examples of the other additive include a halation preventing agent, a storage stabilizing agent, a defoaming agent and a shape improving agent. Specific examples thereof include 4-hydroxy-4′-methylchalkone.

### [Resist Pattern Formation Method]

The resist pattern formation method of the present embodiment comprises:
a step of forming a resist film on a substrate using the composition for film formation of the present embodiment;
a step of exposing a pattern on the resist film; and
a step of subjecting the resist film after exposure to a development treatment.

The composition for film formation of the present embodiment at least contains at least one selected from the compound A and the polymer of the present embodiment, as mentioned above.

Examples of the coating method in the step of forming a resist film include, but are not particularly limited to, spin coating, dip coating, and roll coating. Examples of the substrate include, but are not particularly limited to, a silicon wafer, metal, plastic, glass, and ceramic. After formation of the resist film, a heat treatment may be carried out at a temperature of about 50° C. to 200° C. The film thickness of the resist film is not particularly limited and is for example, 50 nm to 1 μm.

In the exposure step, exposure may be carried out via a predetermined mask pattern, or shot exposure may be carried out without masking. The thickness of the coating film is, for example, 0.1 to 20 μm, and preferably about 0.3 to 2 μm. Lights of various wavelengths such as ultraviolet ray, X-ray can be utilized for exposure, and for example, far ultraviolet ray such as F2 excimer laser (wavelength: 157 nm), ArF excimer laser (wavelength: 193 nm), and KrF excimer laser (wavelength: 248 nm), extreme ultraviolet ray (wavelength: 13 nm), X-ray, and electron beam can be arbitrarily selected and used, as a light source. Among them, extreme ultraviolet ray is preferable. The exposure conditions such as the exposure amount are arbitrarily selected depending on the composition of the above resin and/or compound blended, the kind of each additive, and the like.

In the present embodiment, to stably form a fine pattern with a high degree of accuracy, the resist film is preferably subjected to a heat treatment at a temperature of 50 to 200° C. for 30 seconds or more, after exposure. In this case, variation of sensitivity depending on the kind of substrate may increase at a temperature lower than 50° C. Thereafter, development is carried out with an alkaline developing solution under the conditions of normally at 10 to 50° C. for 10 to 200 seconds, and preferably 20 to 25° C. for 15 to 90 seconds, resulting in formation of a predetermined resist pattern.

The above alkaline developing solution used is an alkaline aqueous solution in which an alkaline compound such as an alkali metal hydroxide, aqueous ammonia, an alkylamine, an alkanolamine, a heterocyclic amine, a tetraalkylammonium hydroxide, choline, 1,8-diazabicyclo-[5.4.0]-7-undecene, and 1,5-diazabicyclo-[4.3.0]-5-nonene is dissolved such that the concentration is normally 1 to 10% by mass and preferably 1 to 3% by mass. Into the above developing solution comprising the alkaline aqueous solution, a water-soluble organic solvent and a surfactant can be arbitrarily added.

The composition of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

Also, the composition of the present embodiment can be used as a patterning material for lithography applications. For lithography process applications, the composition can be used for various applications such as a semiconductor, a liquid crystal display panel, a display panel using OLED, a power device, and CCD and other sensors. In particular, for semiconductors and integrated circuits on devices, the composition of the present embodiment can be suitably utilized in the process of forming a device element on a silicon wafer, for the purpose of constructing a semiconductor element and other devices by forming a pattern on an insulating film on the substrate side by etching based on the pattern formed on the upper surface side of the insulating layer such as a silicon oxide film and other oxide films utilizing the composition of the present embodiment, further laminating a metal film or a semiconductor material based on the formed insulating film pattern, and forming a circuit pattern.

### EXAMPLES

The present embodiment will be described in more detail with reference to Examples and Comparative Examples below. However, the present embodiment is not limited to these Examples. The measurement methods used in the present embodiments are as follows.

### [Measurement Method]

### [Nuclear Magnetic Resonance (NMR)]

The structure of the compound was verified by carrying out NMR measurement under the following conditions using a nucleus magnetic resonance apparatus “Advance 600 II spectrometer” (product name, manufactured by Bruker).

### [¹H-NMR Measurement]

Frequency: 400 MHz
Solvent: CDCl3, or d6-DMSO
Internal standard: TMS
Measurement temperature: 23° C.

### (Molecular Weight)

The molecular weight of a compound was measured by LC-MS analysis using Acquity UPLC/MALDI-Synapt HDMS manufactured by Waters Corp.

### (Lc Purity)

Gel permeation chromatography (GPC) analysis was carried out under the following conditions, and the LC purity was calculated from the area fraction of the GC chart and the peak intensity ratio between the target peak and the reference peak.
LC measurement apparatus: Nexera-i LC-2040C manufactured by Shimadzu Corporation
Column: Waters XBridge BEH C18 2.5 um 3.0×75 mm
Column temperature: 40° C.
Mobile phase solvent: pure water/acetonitrile (acetonitrile 70%)
Gradient conditions: the following Table

TABLE 1

| | Acetonitrile/water/phosphoric acid | | | |
|---|---|---|---|---|
| | Time | Ultrapure | Acetonitrile | 1% Aqueous phosphoric |
| Eluent | [min] | water [%] | [%] | acid solution [%] |
| Analysis | 0 | 93 | 5 | 2 |
| method | 1 | 93 | 5 | 2 |
| | 16 | 0 | 98 | 2 |
| | 20 | 0 | 98 | 2 |
| | 20.2 | 93 | 5 | 2 |
| | 24.2 | 93 | 5 | 2 |

Flow rate: 0.7 mL/min
Detection method: UV (detection wavelength: 254 nm or 220 nm)
Sensitivity: 2.5 AUN (AUXRNG6)
Sample: 25 mg/50 mL-acetonitrile, 1.5 um injection

### (Gpc Purity)

The GPC purity was measured using a GPC column APC ACQUITY APC XT45 (150 mm) mounted on a GPC apparatus (Shodex GPC system 11) and an UV detector (measurement wavelength 254 nm or 220 nm). Tetrahydrofuran was used as an eluent. The column temperature was 30° C.

Sample: adjusted to a 0.2 wt. % tetrahydrofuran solution

### Example 1

### Synthesis of Compound Ma1a

The compound Ma1a was synthesized by the following steps 1 to 3.

### (Step 1): Iodination (Synthesis of 4-hydroxy-5-iodo-3-methoxybenzaldehyde)

4-Hydroxy-5-iodo-3-methoxybenzaldehyde was synthesized by introducing a halogen according to the following reaction formula.

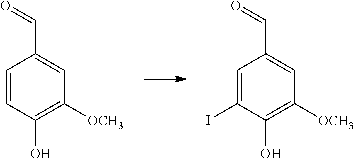 [SMILES: COc1cc(C=O)ccc1O ; COc1cc(C=O)cc(I)c1O]

A 1000 mL glass reaction vessel was charged with 68.4 g (0.450 mol) of 4-hydroxy-3-methoxybenzaldehyde and 297 mL of methanol, and blowing of nitrogen into the reaction vessel at a flow rate of 10 mL/min and stirring were initiated. After dissolution of 4-hydroxy-3-methoxybenzaldehyde was confirmed, the reaction vessel was charged with 137 mL of ion exchanged water and 33.4 g (0.315 mol) of sodium carbonate and stirred at a room temperature of 22° C. for 1 hour. The reaction vessel was charged with 137 g (0.540 mol) of iodine by portionwise addition, and iodine was stirred at a room temperature of 22° C. for 20 hours. After a 16.6% aqueous sulfurous acid sodium solution was added until both the decoloration of the solution and the attainment of a basicity of the system were confirmed, 229 mL of water was added, and the mixture was stirred for 1 hour. The precipitated solid was filtered with a suction filter, rinsed with acetonitrile, reslurried with acetonitrile, and dried to obtain 100 g of a white solid. As a result of the analysis by liquid chromatography-mass spectrometry (LC-MS), the molecular weight was found to be 278. As a result of the analysis by gel permeation chromatography (GPC), the purity at the main peak was 99.8%. The following peaks were found by carrying out ¹H-NMR measurement under the aforementioned measurement conditions, and the compound was confirmed to have the chemical structure of 4-hydroxy-5-iodo-3-methoxybenzaldehyde. The LC purity at 254 nm was 99.8%, and the GPC purity was 99.9%.

δ (ppm) (d6-DMSO): 3.9 (3H, —CH3), 7.4 (1H, Ph), 7.9 (1H, Ph), 9.8 (1H, —CHO), 10.8 (1H, —OH)

### (Step 2): Olefination (Synthesis of Compound Ma1) and Removal of Phosphine Oxide by Acid Addition Method

The compound Ma1 was synthesized by removing phosphine oxide by olefination by a Wittig reaction and a method for adding an acid, according to the following reaction formula.

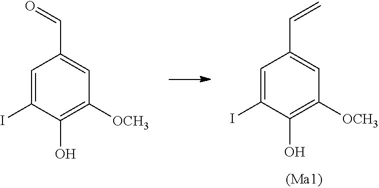

A 1000 mL glass reaction vessel was charged with 134 g (0.375 mol) of methyltriphenylphosphoniumbromide and 348 mL of THF, and blowing of nitrogen into the reaction vessel at a flow rate of 10 mL/min and stirring were initiated. The reaction vessel was immersed in ice water and cooled to 0° C., and charged with 70.1 g (0.625 mol) of potassium tert-butoxide by portionwise addition, and stirring was continued for 30 minutes. The reaction vessel was charged with 69.5 g (0.250 mol) of 4-hydroxy-5-iodo-3-methoxybenzaldehyde obtained in step 1 by portionwise addition and stirred for 1 hour, and then the reaction solution was gradually added to 126 g of 3 M hydrochloric acid put in another glass reaction vessel. The reaction vessel was returned to room temperature, 209 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma1 and triphenylphosphine oxide.

In a 100 mL glass reaction vessel, 13.0 g of sulfuric acid and 82.8 mL of THF were put, and the mixture was stirred at 0° C. to prepare a sulfuric acid solution. A 1000 mL glass reaction vessel was charged with a Wittig reaction solution, immersed in ice water, and stirred while cooling to 0° C. The sulfuric acid solution prepared was added dropwise into the reaction vessel over 10 minutes and stirred for 1 hour. A composite of sulfuric acid-triphenylphosphine oxide precipitated was filtered with a suction filter to obtain a compound Ma1 solution having a reduced content of triphenylphosphine oxide. To the compound Ma1 solution, 69.5 g of silica gel (manufactured by Kanto Chemical Co., Inc., for column chromatography, 60 N (spherical, neutral), particle size: 100 to 210 μm) was added, and the mixture was suspended and stirred at a room temperature of 22° C. for 1 hour. Silica gel was removed by filtering the mixture with a suction filter to obtain a compound Ma1 solution having further reduced contents of phosphine oxide and the polymer. The compound Ma1 solution was concentrated, an excess of heptane was then added thereto, the solution was cooled, and the precipitate obtained was recovered by suction filtration and dried in vacuo at 25° C. to obtain 48.3 g of a white solid. As a result of the analysis by liquid chromatography-mass spectrometry (LC-MS), the molecular weight was found to be 276. The following peaks were found by carrying out ¹H-NMR measurement under the above measurement conditions, and the compound was confirmed to have the chemical structure of the compound Ma1. The LC purity at 254 nm was 99.9%, and the GPC purity was 99.9%.

δ (ppm) (d6-DMSO): 3.8 (3H, —CH3), 5.1 (1H, ═CH2), 5.7 (1H, ═CH2), 6.6 (1H, —CH═), 7.1 (1H, Ph), 7.3 (1H, Ph), 9.6 (1H, —OH)

### (Step 3): Acetyl Protection (Synthesis of Compound Ma1a)

The compound Ma1a was synthesized by introducing an acetyl group according to the following reaction formula.

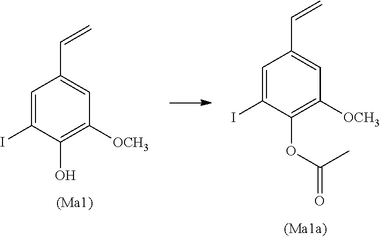

Using a 1000 mL glass flask as a reaction vessel, 41.4 g (0.150 mol) of the compound Ma1 obtained in step 2 was dissolved by using 414 mL of chloroform as a solvent, 22.8 g (0.225 mol) of triethylamine and 2.75 g (0.0225 mol) of 4-dimethylaminopyridine were added thereto, and then the mixture was immersed in ice water and stirred while cooling to 0° C. 23.0 g (0.225 mol) of acetic anhydride was added thereto for 20 minutes, the mixture was stirred for 2 hours under ice cooling, and 126 g of 0.2 M hydrochloric acid was then added dropwise for 20 minutes. 29.6 mL of chloroform was added thereto to separate the mixture, and the organic layer was washed with 5% sodium bicarbonate water and water. The solution was concentrated, an excess of heptane was then added thereto, the solution was cooled, and the obtained precipitate was recovered by suction filtration and dried in vacuo at 25° C. to obtain 38.2 g of a white solid. The white solid sample was analyzed by liquid chromatography-mass spectrometry (LC-MS) and, as a result, the molecular weight was found to be 318, and the white solid was confirmed to be the compound Ma1a.

The following peaks were found by carrying out ¹H-NMR measurement under the above measurement conditions, and the compound was confirmed to have the chemical structure of the compound Ma1a. The LC purity at 254 nm was 99.9%, and the GPC purity was 99.9%.

δ (ppm) (d6-DMSO): 2.3 (3H, —CH3), 3.8 (3H, —CH3), 5.3 (1H, ═CH2), 5.9 (1H, ═CH2), 6.7 (1H, —CH═), 7.3 (1H, Ph), 7.5 (1H, Ph)

### Example 1b

### Synthesis of compound Ma1a

Iodination (synthesis of 4-hydroxy-5-iodo-3-methoxybenzaldehyde) was carried out by the following (step 1 b) instead of (step 1) of Example 1.

### (Step 1 b): Iodination (Synthesis of 4-hydroxy-5-iodo-3-methoxybenzaldehyde)

4-Hydroxy-5-iodo-3-methoxybenzaldehyde was synthesized by introducing a halogen according to the following reaction formula.

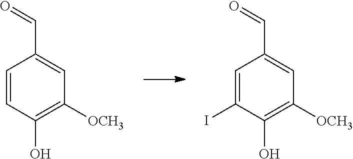 [SMILES: COc1cc(C=O)ccc1O ; COc1cc(C=O)cc(I)c1O]

A 1000 mL glass reaction vessel was charged with 68.4 g (0.450 mol) of 4-hydroxy-3-methoxybenzaldehyde and 297 mL of methanol, and blowing of nitrogen into the reaction vessel at a flow rate of 10 mL/min and stirring were initiated. After dissolution of 4-hydroxy-3-methoxybenzaldehyde was confirmed, the reaction vessel was charged with 137 mL of ion exchanged water and 33.4 g (0.315 mol) of sodium carbonate and stirred at a room temperature of 22° C. for 1 hour. The reaction vessel was charged with 137 g (0.540 mol) of iodine by portionwise addition, and iodine was stirred at a room temperature of 22° C. for 20 hours. After a 16.6% aqueous sulfurous acid sodium solution was added until both the decoloration of the solution and the attainment of a basicity of the system were confirmed, 229 mL of water was added, and the mixture was stirred for 1 hour. The precipitated solid was filtered with a suction filter, rinsed with acetonitrile, and dried to obtain 100 g of a white solid. As a result of the analysis by liquid chromatography-mass spectrometry (LC-MS), the molecular weight was found to be 278. As a result of the analysis by gel permeation chromatography (GPC), the purity was confirmed to be 98.1%. The following peaks were found by carrying out ¹H-NMR measurement under the aforementioned measurement conditions, and the compound was confirmed to have the chemical structure of 4-hydroxy-5-iodo-3-methoxybenzaldehyde. The LC purity at 254 nm was 99.8%, and the GPC purity was 99.6%.

δ (ppm) (d6-DMSO): 3.9 (3H, —CH3), 7.4 (1H, Ph), 7.9 (1H, Ph), 9.8 (1H, —CHO), 10.8 (1H, —OH)

### Example 1c

### Synthesis of compound Ma1a

Iodination (synthesis of 4-hydroxy-5-iodo-3-methoxybenzaldehyde) was carried out by the following (step 1 b) instead of (step 1) of Example 1.

### (Step 1 b): Iodination (Synthesis of 4-hydroxy-5-iodo-3-methoxybenzaldehyde)

4-Hydroxy-5-iodo-3-methoxybenzaldehyde was synthesized by introducing a halogen according to the following reaction formula.

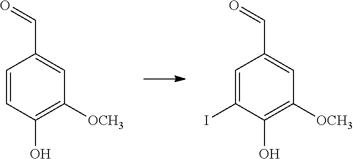 [SMILES: COc1cc(C=O)ccc1O ; COc1cc(C=O)cc(I)c1O]

A 1000 mL glass reaction vessel was charged with 68.4 g (0.450 mol) of 4-hydroxy-3-methoxybenzaldehyde and 297 mL of methanol, and blowing of nitrogen into the reaction vessel at a flow rate of 10 mL/min and stirring were initiated. After dissolution of 4-hydroxy-3-methoxybenzaldehyde was confirmed, the reaction vessel was charged with 137 mL of ion exchanged water and 33.4 g (0.315 mol) of sodium carbonate and stirred at a room temperature of 22° C. for 1 hour. The reaction vessel was charged with 137 g (0.540 mol) of iodine by portionwise addition, and iodine was stirred at a room temperature of 22° C. for 20 hours. After a 16.6% aqueous sulfurous acid sodium solution was added until both the decoloration of the solution and the attainment of a basicity of the system were confirmed, 229 mL of water was added, and the mixture was stirred for 1 hour. The precipitated solid was filtered with a suction filter, and rinsed with acetonitrile. The obtained white solid was dissolved in 600 mL of THF containing no stabilizing agent, 100 g of silica gel (manufactured by Kanto Chemical Co., Inc. 60 N) was then added, the mixture was stirred at an internal temperature of 25° C. for 2 hours, and the filtrate was then recovered by filtration. Then, 100 mL of a 1% aqueous sodium hydrogen sulfite solution was added thereto, the mixture was stirred for 30 minutes, and the aqueous phase was discharged and then concentrated by concentration under reduced pressure so that a solid content of about 50% was achieved. After 1 L of hexane was added thereto, the precipitate was recovered and further dried to obtain 87 g of a white solid. As a result of the analysis by liquid chromatography-mass spectrometry (LC-MS), the molecular weight was found to be 278. As a result of the analysis by gel permeation chromatography (GPC), the purity was confirmed to be 99.8%. The following peaks were found by carrying out ¹H-NMR measurement under the aforementioned measurement conditions, and the compound was confirmed to have the chemical structure of 4-hydroxy-5-iodo-3-methoxybenzaldehyde. The LC purity at 254 nm was 99.8%, and the GPC purity was 99.9%.

δ (ppm) (d6-DMSO): 3.9 (3H, —CH3), 7.4 (1H, Ph), 7.9 (1H, Ph), 9.8 (1H, —CHO), 10.8 (1H, —OH)

### Example 2

### Synthesis of Compound Ma1a

The compound Ma1a was synthesized by the following steps 1 to 3.

### (Step 1): Iodination

4-Hydroxy-5-iodo-3-methoxybenzaldehyde was synthesized by the same operation as in Example 1 (step 1).

### (Step 2): Acetyl protection (Synthesis of 4-acetoxy-5-iodo-3-methoxybenzaldehyde)

4-Acetoxy-5-iodo-3-methoxybenzaldehyde was synthesized by introducing an acetyl group according to the following reaction formula.

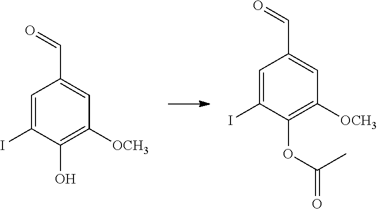

Using a 1000 mL glass flask as a reaction vessel, 55.6 g (0.200 mol) of 4-hydroxy-5-iodo-3-methoxybenzaldehyde obtained in step 1 was dissolved by using 556 mL of chloroform as a solvent, 30.4 g (0.300 mol) of triethylamine and 3.67 g (0.0300 mol) of 4-dimethylaminopyridine were then added thereto, and then the mixture was immersed in ice water and stirred while cooling to 0° C. 30.6 g (0.300 mol) of acetic anhydride was added thereto for 20 minutes, the mixture was stirred for 2 hours under ice cooling, and 238 g of 0.2 M hydrochloric acid was then added dropwise for 20 minutes. 39.7 mL of chloroform was added thereto to separate the mixture, and the organic layer was washed with 5% sodium bicarbonate water and water. The solution was concentrated, an excess of heptane was then added thereto, the solution was cooled, and the obtained precipitate was recovered by suction filtration and dried in vacuo at 25° C. to obtain 51.2 g of a white solid. The white solid sample was analyzed by liquid chromatography-mass spectrometry (LC-MS) and, as a result, the molecular weight was found to be 320 and the white solid was confirmed to be 4-acetoxy-5-iodo-3-methoxybenzaldehyde.

The following peaks were found by carrying out ¹H-NMR measurement under the above measurement conditions, and the compound was confirmed to have the chemical structure of 4-acetoxy-5-iodo-3-methoxybenzaldehyde. The LC purity at 254 nm was 99.9%, and the GPC purity was 99.9%.

δ (ppm) (d6-DMSO): 2.3 (3H, —CH3), 3.8 (3H, —CH3), 7.3 (1H, Ph), 7.9 (1H, Ph), 9.9 (1H, —CHO)

### (Step 3): Olefination and Removal of Phosphine Oxide by Addition of Acid

The compound Ma1a was synthesized by removing phosphine oxide by olefination by a Wittig reaction and a method for adding an acid, according to the following reaction formula.

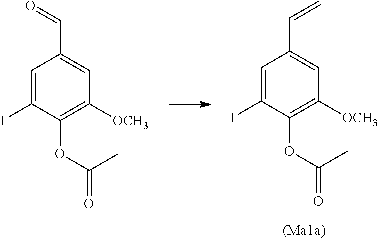

A 1000 mL glass reaction vessel was charged with 80.4 g (0.225 mol) of methyltriphenylphosphoniumbromide and 240 mL of THF, and blowing of nitrogen into the reaction vessel at a flow rate of 10 mL/min and stirring were initiated. The reaction vessel was immersed in ice water and cooled to 0° C., and charged with 42.1 g (0.375 mol) of potassium tert-butoxide by portionwise addition, and stirring was continued for 30 minutes. The reaction vessel was charged with 48.0 g (0.150 mol) of 4-acetoxy-5-iodo-3-methoxybenzaldehyde obtained in step 2 by portionwise addition and stirred for 1 hour, and then the reaction solution was gradually added to 87.0 g of 3 M hydrochloric acid put in another glass reaction vessel. The reaction vessel was returned to room temperature, 144 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma1a and triphenylphosphine oxide.

In a 100 mL glass reaction vessel, 7.80 g of sulfuric acid and 44.2 mL of tetrahydrofuran (THF) were put, and the mixture was stirred at 0° C. to prepare a sulfuric acid solution. A 1000 mL glass reaction vessel was charged with a Wittig reaction solution, immersed in ice water, and stirred while cooling to 0° C. The sulfuric acid solution prepared was added dropwise into the reaction vessel over 10 minutes and stirred for 1 hour. The composite of sulfuric acid-triphenylphosphine oxide precipitated was filtered with a suction filter to obtain a compound Mala solution having a reduced content of triphenylphosphine oxide. To the compound Ma1a solution, 48.0 g of silica gel (manufactured by Kanto Chemical Co., Inc., for column chromatography, 60 N (spherical, neutral), particle size: 100 to 210 μm) was added, and the mixture was suspended and stirred at a room temperature of 22° C. for 1 hour. Silica gel was removed by filtering the mixture with a suction filter to obtain a compound Ma1a solution having further reduced contents of phosphine oxide and the polymer. The compound Ma1a solution was concentrated, an excess of heptane was then added thereto, the solution was cooled, and the obtained precipitate was recovered by suction filtration and dried in vacuo at 25° C. to obtain 33.4 g of a white solid. As a result of the analysis by liquid chromatography-mass spectrometry (LC-MS), the molecular weight was found to be 318. The following peaks were found by carrying out ¹H-NMR measurement under the above measurement conditions, and the compound was confirmed to have the chemical structure of the compound Ma1a. The LC purity at 254 nm was 99.9%, and the GPC purity was 99.9%.

δ (ppm) (d6-DMSO): 2.3 (3H, —CH3), 3.8 (3H, —CH3), 5.3 (1H, ═CH2), 5.9 (1H, ═CH2), 6.7 (1H, —CH═), 7.3 (1H, Ph), 7.5 (1H, Ph)

### Example 3

### Synthesis of Compound Ma1a (Removal of Phosphine Oxide by Crystallization)

The compound Ma1a was synthesized by steps 1 to 3.

### (Step 1): Iodination

4-Hydroxy-5-iodo-3-methoxybenzaldehyde was synthesized by the same operation as in Example 1 (step 1).

### (Step 2): Acetyl Protection

4-Acetoxy-5-iodo-3-methoxybenzaldehyde was obtained by the same operation as in Example 2 (step 2).

### (Step 3): Olefination and Removal of Phosphine Oxide by Crystallization

The compound Ma1a was synthesized by removing phosphine oxide by olefination by a Wittig reaction and crystallization, according to the following reaction formula.

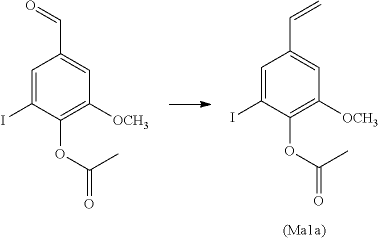

A 1000 mL glass reaction vessel was charged with 80.4 g (0.225 mol) of methyltriphenylphosphoniumbromide and 240 mL of THF, and blowing of nitrogen into the reaction vessel at a flow rate of 10 mL/min and stirring were initiated. The reaction vessel was immersed in ice water and cooled to 0° C., and charged with 42.1 g (0.375 mol) of potassium tert-butoxide by portionwise addition, and stirring was continued for 30 minutes. The reaction vessel was charged with 48.0 g (0.150 mol) of 4-acetoxy-5-iodo-3-methoxybenzaldehyde obtained from step 2 by portionwise addition and stirred for 1 hour, and then the reaction solution was gradually added to 87.0 g of 3 M hydrochloric acid put in another glass reaction vessel. The reaction vessel was returned to room temperature, 144 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma1a and triphenylphosphine oxide.

The Wittig reaction solution was concentrated until the concentration of toluene in the solution reached 50% by mass. 632 mL of heptane was gradually added thereto, and the mixture was stirred for 1 hour. The precipitated solid was filtered with a suction filter to obtain a compound Ma1a solution having a reduced content of triphenylphosphine oxide. The compound Ma1a solution was concentrated, an excess of heptane was then added thereto, the solution was cooled, and the obtained precipitate was recovered by suction filtration and dried in vacuo at 25° C. to obtain 28.6 g of a white solid. As a result of the analysis by liquid chromatography-mass spectrometry (LC-MS), the molecular weight was found to be 318. The following peaks were found by carrying out ¹H-NMR measurement under the above measurement conditions, and the compound was confirmed to have the chemical structure of the compound Ma1a. The LC purity at 254 nm was 99.9%, and the GPC purity was 99.9%.

δ (ppm) (d6-DMSO): 2.3 (3H, —CH3), 3.8 (3H, —CH3), 5.3 (1H, ═CH2), 5.9 (1H, ═CH2), 6.7 (1H, —CH═), 7.3 (1H, Ph), 7.5 (1H, Ph)

### Example 4

### Synthesis of Compound Ma1 (Removal of Phosphine Oxide Using Acetic Acid)

The compound Ma1 was synthesized by the following steps 1 to 2.

### (Step 1): Iodination

4-Hydroxy-5-iodo-3-methoxybenzaldehyde was obtained by the same operation as in Example 1 (step 1).

### (Step 2): Olefination and Removal of Phosphine Oxide by Acid Addition Method

47.2 g of the compound Ma1 was obtained by the method described in Example 1 (step 2), except for using an acetic acid solution of 33.0 g of acetic acid and 187 mL of THF instead of the sulfuric acid solution of 13.0 g of sulfuric acid and 82.8 mL of THF. The LC purity at 254 nm was 99.9%, and the GPC purity was 99.9%.

### Example 5

### Synthesis of Compound Ma1 (Removal of Phosphine Oxide Using Acetic Acid)

The compound Ma1 was synthesized by steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Acid Addition Method

45.6 g of the compound Ma1 was obtained by the method described in Example 1 (step 2), except for using 75.1 g of acetic acid instead of the sulfuric acid solution of 13.0 g of sulfuric acid and 82.8 mL of THF. The LC purity at 254 nm was 99.9%, and the GPC purity was 99.7%.

### Example 6

### Synthesis of Compound Ma1 (Removal of Phosphine Oxide Using Triphenylmethanol)

The compound Ma1 was synthesized by the following steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Acid Addition Method

49.0 g of the compound Ma1 was obtained by the method described in Example 1 (step 2), except for using a triphenylmethanol solution of 66.4 g of triphenylmethanol and 376 mL of THF instead of the sulfuric acid solution of 13.0 g of sulfuric acid and 82.8 mL of THF. The LC purity at 254 nm was 99.6%, and the GPC purity was 99.9%.

### Example 7

### Synthesis of Compound Ma1 (Change of Extraction Solvent)

The compound Ma1 was synthesized by the following steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Acid Addition Method

49.1 g of the compound Ma1 was obtained by the method described in Example 1 (step 2), except for using 209 mL of butyl acetate instead of 209 mL of toluene as an extraction solvent. The LC purity at 254 nm was 99.9%, and the GPC purity was 99.9%.

### Example 8

### Synthesis of Compound Ma1 (Change of Extraction Solvent)

The compound Ma1 was synthesized by the following steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Acid Addition Method

48.9 g of the compound Ma1 was obtained by the method described in Example 1 (step 2), except for using 209 mL of cyclopentyl methyl ether instead of 209 mL of toluene as an extraction solvent. The LC purity at 254 nm was 99.8%, and the GPC purity was 99.9%.

### Example 9

### Synthesis of Compound Ma1 (Removal of Phosphine Oxide Using Sulfuric Acid)

The compound Ma1 was synthesized by the following steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Acid Addition Method

41.8 g of the compound Ma1 was obtained by the method described in Example 1 (step 2), except for using a sulfuric acid solution of 6.13 g of sulfuric acid and 34.7 mL of THF instead of the sulfuric acid solution of 13.0 g of sulfuric acid and 82.8 mL of THF, and 174 g of silica gel instead of 69.5 g of silica gel. The LC purity at 254 nm was 99.9%, and the GPC purity was 99.9%.

### Example 10

### Synthesis of Compound Ma2a

The compound Ma2a was synthesized by the following steps 1 to 3.

### (Step 1): Iodination (Synthesis of 4-hydroxy-3,5-diiodo-benzaldehyde)

4-Hydroxy-3,5-diiodo-benzaldehyde was synthesized by introducing a halogen according to the following reaction formula.

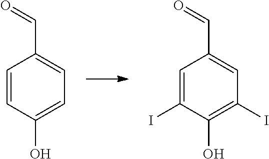 [SMILES: O=Cc1ccc(O)cc1 ; O=Cc1cc(I)c(O)c(I)c1]

A 2000 mL glass reaction vessel was charged with 68.4 g (0.450 mol) of 4-hydroxy-benzaldehyde and 600 mL of methanol, and blowing of nitrogen into the reaction vessel at a flow rate of 10 mL/min and stirring were initiated. After dissolution of 4-hydroxy-benzaldehyde was confirmed, the reaction vessel was charged with 275 mL of ion exchanged water and 133.6 g (1.26 mol) of sodium carbonate and stirred at a room temperature of 22° C. for 1 hour. The reaction vessel was charged with 137 g (0.540 mol) of iodine by portionwise addition, and iodine was stirred at a room temperature of 22° C. for 20 hours. After a 16.6% aqueous sulfurous acid sodium solution was added until both the decoloration of the solution and the attainment of a basicity of the system were confirmed, 460 mL of water was added, and the mixture was stirred for 1 hour. The precipitated solid was filtered with a suction filter, rinsed, reslurried, and dried to obtain 135 g of a white solid. As a result of the analysis by liquid chromatography-mass spectrometry (LC-MS), the molecular weight was found to be 374. The following peaks were found by carrying out ¹H-NMR measurement under the aforementioned measurement conditions, and the compound was confirmed to have the chemical structure of 4-hydroxy-3,5-diiodo-benzaldehyde. The LC purity at 254 nm was 99.8%, and the GPC purity was 99.9%.

δ (ppm) (d6-DMSO): 8.0 (2H, Ph), 9.9 (1H, —CHO), 9.6 (1H, —OH)

### (Step 2): Olefination and Removal of Phosphine Oxide by Addition of Acid (Synthesis of Compound Ma2)

The compound Ma2 was synthesized by removing phosphine oxide by olefination by a Wittig reaction and a method for adding an acid, according to the following reaction formula.

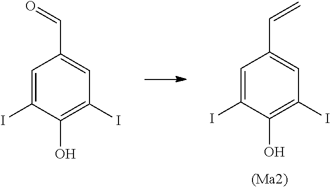

A 1000 mL glass reaction vessel was charged with 134 g (0.375 mol) of methyltriphenylphosphoniumbromide and 348 mL of THF, and blowing of nitrogen into the reaction vessel at a flow rate of 10 mL/min and stirring were initiated. The reaction vessel was immersed in ice water and cooled to 0° C., and charged with 70.1 g (0.625 mol) of potassium tert-butoxide by portionwise addition, and stirring was continued for 30 minutes. The reaction vessel was charged with 93.5 g (0.250 mol) of 4-hydroxy-3,5-diiodo-benzaldehyde obtained from step 1 by portionwise addition and stirred for 1 hour, and then the reaction solution was gradually added to 126 g of 3 M hydrochloric acid put in another glass reaction vessel. The reaction vessel was returned to room temperature, 209 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma2 and triphenylphosphine oxide.

In a 100 mL glass reaction vessel, 13.0 g of sulfuric acid and 82.8 mL of THF were put, and the mixture was stirred at 0° C. to prepare a sulfuric acid solution. A 1000 mL glass reaction vessel was charged with a Wittig reaction solution, immersed in ice water, and stirred while cooling to 0° C. The sulfuric acid solution prepared was added dropwise into the reaction vessel over 10 minutes and stirred for 1 hour. A composite of sulfuric acid-triphenylphosphine oxide precipitated was filtered with a suction filter to obtain a compound Ma2 solution having a reduced content of triphenylphosphine oxide. To the compound Ma2 solution, 69.5 g of silica gel (manufactured by Kanto Chemical Co., Inc., for column chromatography, 60 N (spherical, neutral), particle size: 100 to 210 μm) was added, and the mixture was suspended and stirred at a room temperature of 22° C. for 1 hour. Silica gel was removed by filtering the mixture with a suction filter to obtain a compound Ma2 solution having reduced contents of phosphine oxide and the polymer. The compound Ma2 solution was concentrated, an excess of heptane was then added thereto, the solution was cooled, and the obtained precipitate was recovered by suction filtration and dried in vacuo at 25° C. to obtain 65.1 g of a white solid. As a result of the analysis by liquid chromatography-mass spectrometry (LC-MS), the molecular weight was found to be 372. The following peaks were found by carrying out ¹H-NMR measurement under the aforementioned measurement conditions, and the compound was confirmed to have the chemical structure of the compound Ma2. The LC purity at 254 nm was 99.9%, and the GPC purity was 99.9%.

δ (ppm) (d6-DMSO): 5.2 (1H, ═CH2), 5.7 (1H, ═CH2), 6.7 (1H, —CH═), 7.5 (2H, Ph), 9.6 (1H, —OH)

### (Step 3): Acetyl Protection (Synthesis of Compound Ma2a)

The compound Ma2a was synthesized by introducing an acetyl group according to the following reaction formula.

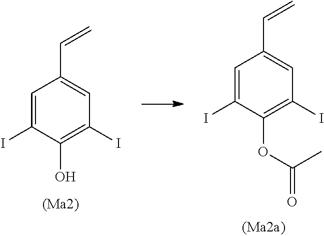

Using a 1000 mL glass flask as a reaction vessel, 55.8 g (0.150 mol) of the compound Ma2 was dissolved by using 414 mL of chloroform as a solvent, 22.8 g (0.225 mol) of triethylamine and 2.75 g (0.0225 mol) of 4-dimethylaminopyridine were added thereto, and then the mixture was immersed in ice water and stirred while cooling to 0° C. 23.0 g (0.225 mol) of acetic anhydride was added thereto for 20 minutes, the mixture was stirred for 2 hours under ice cooling, and 126 g of 0.2 M hydrochloric acid was then added dropwise for 20 minutes. 29.6 mL of chloroform was added thereto to separate the mixture, and the organic layer was washed with 5% sodium bicarbonate water and water. The solution was concentrated, an excess of heptane was then added thereto, the solution was cooled, and the obtained precipitate was recovered by suction filtration and dried in vacuo at 25° C. to obtain 48.7 g of a white solid. The white solid sample was analyzed by liquid chromatography-mass spectrometry (LC-MS) and, as a result, the molecular weight was found to be 414, and the white solid was confirmed to be the compound Ma2a.

The following peaks were found by carrying out ¹H-NMR measurement under the aforementioned measurement conditions, and the compound was confirmed to have the chemical structure of the compound Ma1a. The LC purity at 254 nm was 99.9%, and the GPC purity was 99.9%.

δ (ppm) (d6-DMSO): 2.3 (3H, —CH3), 5.3 (1H, ═CH2), 5.7 (1H, ═CH2), 6.7 (1H, —CH═), 7.6 (2H, Ph)

### Example 11

### Synthesis of Compound Ma1 (Removal of Phosphine Oxide Using Zinc Chloride)

The compound Ma1 was synthesized by the following steps 1 to 2.

### (Step 1): Iodination

4-hydroxy-5-iodo-3-methoxybenzaldehyde was obtained by the same operation as in Example 1 (step 1).

### (Step 2): Olefination and Removal of Phosphine Oxide by Zinc Chloride Addition Method

The compound Ma1 was synthesized by removing phosphine oxide by olefination by a Wittig reaction and a method for adding zinc chloride, according to the following reaction formula.

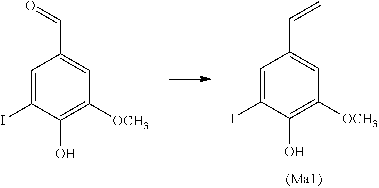

A 1000 mL glass reaction vessel was charged with 134 g (0.375 mol) of methyltriphenylphosphoniumbromide and 348 mL of THF, and blowing of nitrogen into the reaction vessel at a flow rate of 10 mL/min and stirring were initiated. The reaction vessel was immersed in ice water and cooled to 0° C., and charged with 70.1 g (0.625 mol) of potassium tert-butoxide by portionwise addition, and stirring was continued for 30 minutes. The reaction vessel was charged with 69.5 g (0.250 mol) of 4-hydroxy-5-iodo-3-methoxybenzaldehyde obtained from step 1 by portionwise addition and stirred for 1 hour, and then the reaction solution was gradually added to 126 g of 3 M hydrochloric acid put in another glass reaction vessel. The reaction vessel was returned to room temperature, 209 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma1 and triphenylphosphine oxide.

The Wittig reaction solution was concentrated to dryness, and the solid obtained was dissolved in 700 mL of ethanol to obtain a solution of a reaction crude in ethanol. A 2000 mL glass reaction vessel was charged with the solution of a reaction crude in ethanol, immersed in ice water, and stirred while cooling to 0° C. 700 mL of a 10% by mass solution of zinc chloride in ethanol prepared separately was added dropwise into the reaction vessel over 10 minutes, and the mixture was stirred for 1 hour. A composite of zinc chloride-triphenylphosphine oxide precipitated was filtered with a suction filter. The filtrate obtained was concentrated to dryness and then dissolved in toluene, 69.5 g of silica gel (manufactured by Kanto Chemical Co., Inc., for column chromatography, 60 N (spherical, neutral), particle size: 100 to 210 μm) was added, and the mixture was suspended and stirred at a room temperature of 22° C. for 1 hour. Silica gel was removed by filtering the mixture with a suction filter to obtain a compound Ma1 solution having reduced contents of phosphine oxide and the polymer. The compound Ma1 solution was concentrated, an excess of heptane was then added thereto, the solution was cooled, and the obtained precipitate was recovered by suction filtration and dried in vacuo at 25° C. to obtain 9.95 g of a white solid. As a result of the analysis by liquid chromatography-mass spectrometry (LC-MS), the molecular weight was found to be 276. The following peaks were found by carrying out ¹H-NMR measurement under the above measurement conditions, and the compound was confirmed to have the chemical structure of the compound Ma1. The LC purity at 254 nm was 83.0% (triphenylphosphine oxide was 15.2%), and the GPC purity was 82.3%.

δ (ppm) (d6-DMSO): 3.8 (3H, —CH3), 5.1 (1H, ═CH2), 5.7 (1H, ═CH2), 6.6 (1H, —CH═), 7.1 (1H, Ph), 7.3 (1H, Ph), 9.6 (1H, —OH)

From the results of Examples 1 to 10 and Example 11, the removability of phosphine oxide was improved and the target compound was obtained in a higher purity by using Bronsted acid instead of zinc chloride in order to remove phosphine oxide. In addition, in Examples 1 to 10, the polymerization of the compound having an unsaturated double bond was suppressed, so that the target compound was obtained in a higher yield and a higher purity.

### Example 12

### Synthesis of 4-hydroxy-3,5-diiodostyrene

4-Hydroxy-3,5-diiodostyrene was synthesized according to the following steps 1 to 5.

### (Step 1): Iodination (Synthesis of 4-Hydroxy-3,5-Diiodobenzyl Alcohol) 4-Hydroxy-3,5-Diiodobenzyl Alcohol was Synthesized by Introducing a Halogen According to the Following Reaction Formula

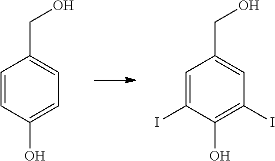 [SMILES: OCc1ccc(O)cc1 ; OCc1cc(I)c(O)c(I)c1]

Using a 2 L glass flask as a reaction vessel, 55.2 g (0.4 mol) of 4-hydroxybenzyl alcohol was dissolved using butanol as a solvent, a 20% by mass aqueous iodine chloride solution (812 g, 1.0 mol) was added dropwise thereto at 50° C. over 120 minutes, and then the mixture was stirred at 50° C. for 2 hours to allow 4-hydroxybenzalcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 148 g of a white solid. The white solid sample was analyzed by liquid chromatography-mass spectrometry (LC-MS) and, as a result, confirmed to be 4-hydroxy-3,5-diiodobenzyl alcohol.

### (Step 2): Oxidation Reaction (Synthesis of 4-Hydroxy-3,5-Diiodo-Benzaldehyde)

4-Hydroxy-3,5-diiodo-benzaldehyde was synthesized by an oxidation reaction according to the following reaction formula.

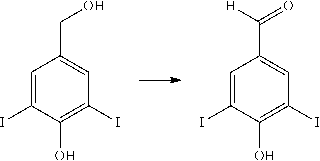 [SMILES: OCc1cc(I)c(O)c(I)c1 ; O=Cc1cc(I)c(O)c(I)c1]

MnO₂ (34 g, 0.4 mol) was added to a methylene chloride solvent and stirred, which was then stirred for 1 hour while a 50% by mass solution in which the total amount of the 4-hydroxy-3,5-diiodobenzyl alcohol obtained from step 1 was dissolved in methylene chloride was added dropwise and then stirred at room temperature for 4 hours, the reaction solution was filtered off, and the solvent was distilled off to obtain 146 g of 4-hydroxy-3,5-diiodobenzaldehyde.

### (Step 3): Malonic Acid Addition Reaction (Synthesis of Compound M8-CINMe)

The compound M8-CINMe was synthesized by an malonic acid addition reaction according to the following reaction formula.

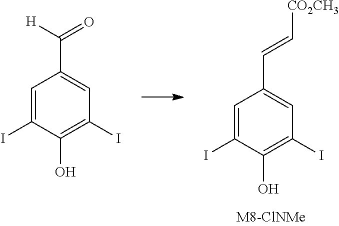 [SMILES: O=Cc1cc(I)c(O)c(I)c1 ; COC(=O)/C=C/c1cc(I)c(O)c(I)c1]

Using a 2 L eggplant flask equipped with a Dean-Stark reflux tube, 146 g (0.38 mol) of 3,5-diiodo4-hydroxybenzaldehyde was mixed with dimethyl malonate (106 g, 0.8 mol), piperidine (34 g, 0.4 mol), acetic acid (24 g, 0.4 mol), and 400 mL of benzene, and allowed to react for 5 hours under reflux conditions. The obtained reaction solution was washed with 200 mL of a 5% by mass aqueous HCl solution, and then washed with a 5% aqueous NaHCO₃ solution. The obtained organic layer was dried over magnesium sulfate, and then the filtrate obtained by filtration was concentrated under reduced pressure to obtain 155 g of the compound M8-CINMe.

### (Step 4): Hydrolysis Reaction (Synthesis of Compound M8-CIN)

The compound M8-CIN was synthesized by a hydrolysis reaction according to the following reaction formula.

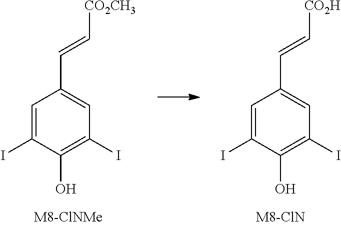 [SMILES: COC(=O)/C=C/c1cc(I)c(O)c(I)c1 ; O=C(O)/C=C/c1cc(I)c(O)c(I)c1]

Using a 20 L eggplant flask equipped with a reflux tube, hydrochloric acid (6N, 1240 mL), and acetic acid (1240 mL) were added to 36 mmol of the compound M8-CINMe obtained above, and the mixture was refluxed for 48 hours. Thereafter, 6 M, 5 L of NaOH aq. was added, and the mixture was extracted with 2500 mL of ethyl acetate to recover the organic layer consisting of ethyl acetate. The obtained organic layer was subjected to a dehydration treatment with magnesium sulfate, and the filtrate filtered thereafter was concentrated under reduced pressure to obtain 146 g of the compound M8-CIN.

### (Step 5): Decarboxylation Reaction (Synthesis of 4-Hydroxy-3,5-Diiodostyrene)

4-Hydroxy-3,5-diiodostyrene was synthesized by any of the methods of step 5-1 to step 5-5, each of which uses a fluoride source, according to the following reaction formula.

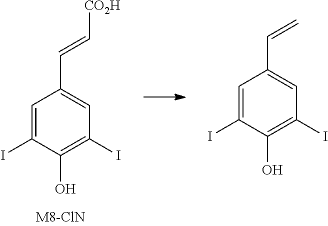

### (Step 5-1): Synthesis of 4-Hydroxy-3,5-Diiodostyrene

Using a 3 L eggplant flask, a solution in which 1.3 g (4 mol) of tetrabutylammonium fluoride trihydrate was dissolved in 200 mL of gamma butyrolactone was slowly added to a solution in which 0.4 mol of M8-CIN obtained in step 4 was dissolved in 400 mL of gamma butyrolactone at 10° C. and stirred, and then the mixture was warmed to 40° C. and stirred for 12 hours. The obtained reaction solution was washed three times with 200 mL of pure water, dried over magnesium sulfate, and the filtrate obtained after filtration was concentrated under reduced pressure to obtain 142 g of a white solid.

As a result of the analysis by liquid chromatography-mass spectrometry (LC-MS), the molecular weight was found to be 372. The following peaks were found by carrying out 1H-NMR measurement under the above measurement conditions, and the compound was confirmed to have the chemical structure of 4-hydroxy-3,5-diiodostyrene. The LC purity at 254 nm was 99.9%, and the GPC purity was 99.9%.

δ (ppm) (d6-DMSO): 5.2 (1H, ═CH2), 5.7 (1H, ═CH2), 6.7 (1H, —CH═), 7.5 (2H, Ph), 9.6 (1H, —OH)

### (Step 5-2): Decarboxylation Reaction (Synthesis of 4-Hydroxy-3,5-Diiodostyrene)

Using a 3 L eggplant flask, a solution in which 1.3 g (4 mol) of tetrabutylammonium fluoride trihydrate was dissolved in 200 mL of dimethylformamide was slowly added to a solution in which 0.4 mol of M8-CIN obtained in step 4 was dissolved in 400 mL of dimethylformamide at 10° C. and stirred, and then the mixture was warmed to 40° C. and stirred for 12 hours. The obtained reaction solution was washed three times with 200 mL of pure water, dried over magnesium sulfate, and the filtrate obtained after filtration was concentrated under reduced pressure to obtain 143 g of a white solid. The compound obtained was analyzed in the same manner as in (step 5-1) and confirmed to have the chemical structure of 4-hydroxy-3,5-diiodostyrene. The LC purity at 254 nm was 99.9%, and the GPC purity was 99.9%.

### (Step 5-3): Decarboxylation Reaction: Synthesis of 4-Hydroxy-3,5-Diiodostyrene

Using a 3 L eggplant flask, a solution in which 1.3 g (4 mol) of tetrabutylammonium fluoride trihydrate was dissolved in a mixed solution of 150 mL of 1,4-dioxane and 50 mL of toluene was slowly added to a solution in which 0.4 mol of M8-CIN obtained in step 4 was dissolved in a mixed solution of 300 mL of 1,4-dioxane and 100 mL of toluene at 10° C. and stirred, and then the mixture was warmed to 40° C. and stirred for 12 hours. The obtained reaction solution was washed three times with 200 mL of pure water, dried over magnesium sulfate, and the filtrate obtained after filtration was concentrated under reduced pressure to obtain 144 g of a white solid. The compound obtained was analyzed in the same manner as in (step 5-1) and confirmed to have the chemical structure of 4-hydroxy-3,5-diiodostyrene. The LC purity at 254 nm was 99.9%, and the GPC purity was 99.9%.

### (Step 5-4): Decarboxylation Reaction (Synthesis of 4-Hydroxy-3,5-Diiodostyrene)

Using a 3 L eggplant flask, a solution in which 1.3 g (4 mol) of tetrabutylammonium fluoride trihydrate was dissolved in a mixed solution of 150 mL of cyclopentyl methyl ether and 50 mL of toluene was slowly added to a solution in which 0.4 mol of M8-CIN obtained in step 4 was dissolved in a mixed solution of 300 mL of cyclopentyl methyl ether and 100 mL of toluene at 10° C. and stirred, and then the mixture was warmed to 40° C. and stirred for 12 hours. The obtained reaction solution was washed three times with 200 mL of pure water, dried over magnesium sulfate, and the filtrate obtained after filtration was concentrated under reduced pressure to obtain 142 g of a white solid. The compound obtained was analyzed in the same manner as in (step 5-1) and confirmed to have the chemical structure of 4-hydroxy-3,5-diiodostyrene. The LC purity at 254 nm was 99.9%, and the GPC purity was 99.9%.

### (Step 5-5): Decarboxylation Reaction (Synthesis of 4-Hydroxy-3,5-Diiodostyrene)

Using a 3 L eggplant flask, a solution in which 1.3 g (4 mol) of tetrabutylammonium fluoride trihydrate was dissolved in a mixed solution of 150 mL of diglyme and 50 mL of toluene was slowly added to a solution in which 0.4 mol of the compound M8-CIN obtained in step 4 was dissolved in a mixed solution of 400 mL of diglyme and 100 mL of toluene at 10° C. and stirred, and then the mixture was warmed to 40° C. and stirred for 12 hours. The obtained reaction solution was washed three times with 200 mL of pure water, dried over magnesium sulfate, and the filtrate obtained after filtration was concentrated under reduced pressure to obtain 141 g of a white solid. The compound obtained was analyzed in the same manner as in (step 5-1) and confirmed to have the chemical structure of 4-hydroxy-3,5-diiodostyrene. The LC purity at 254 nm was 99.9%, and the GPC purity was 99.9%.

### Example 13

### Synthesis of 4-hydroxy-3,5-diiodostyrene

4-Hydroxy-3,5-diiodostyrene was synthesized according to the following step 1 to step 2.

### (Step 1): Iodination (Synthesis of 1-(4-hydroxy-3,5-diiodophenyl)ethanol)

1-(4-Hydroxy-3,5-diiodophenyl)ethanol was synthesized by each method of step 1-1 and step 1-2 according to the following reaction formula.

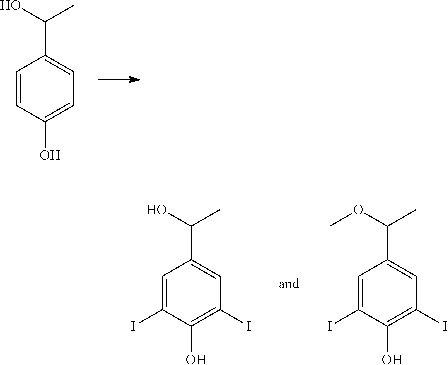

### (Step 1-1): Synthesis of 1-(4-hydroxy-3,5-diiodophenyl)ethanol

A reaction vessel was charged with 119 g of 1-(4-hydroxyphenyl)ethanol, 175 g of iodine, 1400 mL of methanol, and 200 mL of pure water, the reaction vessel was immersed in an ice bath, and stirring was initiated. Subsequently, 87 g of an aqueous iodic acid solution with a mass % concentration of 70 was added dropwise thereto for 60 minutes. Subsequently, the reaction vessel was immersed in a water bath at 25° C., and stirring was continued over 6 hours. Subsequently, 18 mL of an aqueous sodium hydrogen sulfite solution with a mass % concentration of 35 was added to quench the reaction. Subsequently, 3.4 L of pure water was gradually added to the vigorously stirred reaction solution with a dropping funnel and mixed. Subsequently, the precipitate was filtered off with a suction filter. Further, the precipitate was compressed and washed with an aqueous methanol solution with a volume % concentration of 33.3. Subsequently, the precipitate was dried in vacuo at 40° C. to obtain 310 g of a mixture of 1-(4-hydroxy-3,5-diiodophenyl)ethanol and 2,6-diiodo-4-(1-methoxyethyl)phenol. As a result of the HPLC analysis using a UV detector at a measurement wavelength of 254 nm, the ratio between 1-(4-hydroxy-3,5-diiodophenyl)ethanol and 2,6-diiodo-4-(1-methoxyethyl)phenol was 93.4:6.6.

As a result of the analysis by liquid chromatography-mass spectrometry (LC-MS), the molecular weights were found to be 390 and 404, and the substance was confirmed to be a mixture of 1-(4-hydroxy-3,5-diiodophenyl)ethanol and 2,6-diiodo-4-(1-methoxyethyl)phenol.

### (Step 1-2): Synthesis of 1-(4-hydroxy-3,5-diiodophenyl)ethanol

A reaction vessel was charged with 119 g of 1-(4-hydroxyphenyl)ethanol, 177 g of iodine, 1400 mL of methanol, and 350 mL of pure water, the reaction vessel was immersed in an ice bath, and stirring was initiated. Subsequently, 88 g of an aqueous iodic acid solution with a mass % concentration of 70 was added dropwise thereto for 60 minutes. Subsequently, the reaction vessel was immersed in a water bath at 25° C., and stirring was continued over 8 hours. Subsequently, 18 mL of an aqueous sodium hydrogen sulfite solution with a mass % concentration of 35 was added to quench the reaction. Subsequently, 3.5 L of pure water was gradually added to the vigorously stirred reaction solution with a dropping funnel and mixed. Subsequently, the precipitate was filtered off with a suction filter. Further, the precipitate was compressed and then washed with an aqueous methanol solution with a volume % concentration of 33.3. Subsequently, the precipitate was dried in vacuo at 40° C. to obtain 317 g of a mixture of 1-(4-hydroxy-3,5-diiodophenyl)ethanol and 2,6-diiodo-4-(1-methoxyethyl)phenol. As a result of the HPLC analysis using a UV detector at a measurement wavelength of 254 nm, the ratio between 1-(4-hydroxy-3,5-diiodophenyl)ethanol and 2,6-diiodo-4-(1-methoxyethyl)phenol was 85.1:14.9.

As a result of the analysis by liquid chromatography-mass spectrometry (LC-MS), the molecular weights were found to be 390 and 404, and the substance was confirmed to be a mixture of 1-(4-hydroxy-3,5-diiodophenyl)ethanol and 2,6-diiodo-4-(1-methoxyethyl)phenol.

### (Step 2): Synthesis of 4-hydroxy-3,5-diiodostyrene

4-Hydroxy-3,5-diiodostyrene was synthesized by a dehydration reaction using an acid as the catalyst by any of the methods of step 2-1 to step 2-7, according to the following reaction formula.

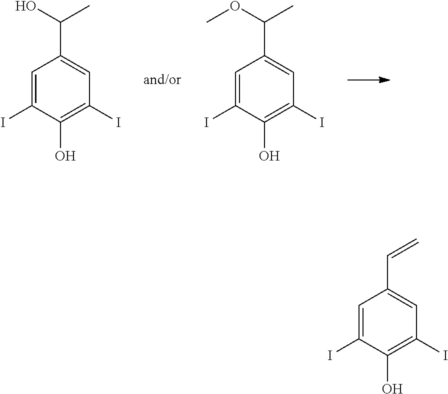

### (Step 2-1): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 29 mL of concentrated sulfuric acid, 0.2 g of 4-hydroxy-2,2,6,6-tetramethylpiperidine 1-oxyl free radical, 0.2 g of 4-methoxyphenol, 2.7 L of dimethylsulfoxide, and 0.3 L of diglyme, and stirring was initiated. Subsequently, the pressure in the reaction vessel was reduced to 30 hPa, the reactor was immersed in a water bath at 90° C., and stirring was continued over 8 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.08:0.01:99.11.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the above measurement conditions.

### (Step 2-2): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 14 g of p-toluenesulfonic acid, 0.2 g of 4-hydroxy-2,2,6,6-tetramethylpiperidine 1-oxyl free radical, 0.2 g of 4-methoxyphenol, 2.5 L of dimethylsulfoxide, and 0.5 L of cyclopentyl methyl ether, and stirring was initiated. Subsequently, the pressure in the reaction vessel was reduced to 30 hPa, the reaction vessel was immersed in a water bath at 90° C., and stirring was continued over 6 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.08:0.01:98.98.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### (Step 2-3): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 29 mL of concentrated sulfuric acid, 0.2 g of 4-hydroxy-2,2,6,6-tetramethylpiperidine 1-oxyl free radical, 0.2 g of 4-methoxyphenol, 22.5 L of dimethylsulfoxide, and 0.5 L of 1,4-dioxane, and stirring was initiated. Subsequently, the pressure in the reaction vessel was reduced to 25 hPa, the reaction vessel was immersed in a water bath at 90° C., and stirring was continued over 8 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.07:0.01:98.75.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### (Step 2-4): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 18 g of p-toluenesulfonic acid, 0.2 g of 4-hydroxy-2,2,6,6-tetramethylpiperidine 1-oxyl free radical, 0.2 g of 4-methoxyphenol, 2.7 L of dimethylsulfoxide, and 0.3 L of toluene, and stirring was initiated. Subsequently, the pressure in the reaction vessel was reduced to 25 hPa, the reaction vessel was immersed in a water bath at 90° C., and stirring was continued over 8 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.07:0.01:99.25.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### (Step 2-5): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 29 mL of concentrated sulfuric acid, 0.2 g of 4-hydroxy-2,2,6,6-tetramethylpiperidine 1-oxyl free radical, 0.2 g of 4-methoxyphenol, 2.5 L of dimethylformamide, and 0.5 L of toluene, and stirring was initiated. Subsequently, the pressure in the reaction vessel was reduced to 20 hPa, the reaction vessel was immersed in a water bath at 90° C., and stirring was continued over 6 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.05:0.01:99.29.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### (Step 2-6): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 29 mL of concentrated sulfuric acid, 0.2 g of 4-hydroxy-2,2,6,6-tetramethylpiperidine 1-oxyl free radical, 0.2 g of 4-methoxyphenol, 2.5 L of dimethylformamide, and 0.5 L of dioxane, and stirring was initiated. Subsequently, the pressure in the reaction vessel was reduced to 25 hPa, the reaction vessel was immersed in a water bath at 90° C., and stirring was continued over 8 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.05:0.01:98.99.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### (Step 2-7): Synthesis of 4-hydroxy-3,5-diiodostyrene

The reaction vessel equipped with a reflux tube and Dean-Stark was charged with 200 g of a mixture having a ratio of 1-(4-hydroxy-3,5-diiodophenyl)ethanol obtained in step 1-1 to 2,6-diiodo-4-(1-methoxyethyl)phenol of 93.4:6.6, 14 g of p-toluenesulfonic acid, 0.2 g of 4-hydroxy-2,2,6,6-tetramethylpiperidine 1-oxyl free radical, 0.2 g of 4-methoxyphenol, 200 g of molecular sieve, 2.5 L of dimethylsulfoxide, and 0.5 L of diisopropyl ether, and stirring was initiated. Subsequently, the reaction vessel was immersed in a water bath at 90° C., and stirring was continued over 6 hours. Subsequently, the reaction vessel was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.08:0.01:98.04.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the aforementioned measurement conditions.

### Example 14

### Synthesis of 3-methoxy-4-acetoxy-5-iodostyrene

### (Step 1): Synthesis of 5-iodovanillin

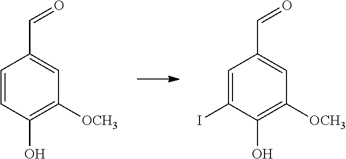 [SMILES: COc1cc(C=O)ccc1O ; COc1cc(C=O)cc(I)c1O]

### (Iodination Step)

A 100 L glass lined reactor vessel and a Teflon-coated TWINSTIR stirring blade were provided, and the reactor vessel was washed with acetone and methanol that is the reaction solvent before reaction, then filled with a 0.1% aqueous nitric acid solution and washed over 18 hours, and sufficiently washed with pure water. The reactor vessel was further connected with a condenser, and the operation of the solution involving stirring in the reactor vessel was conducted in a nitrogen flow (0.1 L/min). Further, the internal temperature was set to 10° C. with a jacket type temperature control mechanism. 5.4 kg of vanillin that is the reaction raw material and 32.4 L of methanol were put in the reactor vessel and stirred under a nitrogen flow (0.1 L/min) at 180 rpm for 30 minutes. Further, 4.9 L of water was added and the mixture was stirred. Then, 6 kg of sodium bicarbonate was added, and then the internal temperature was controlled to be 20° C., and the mixture was stirred for 1 hour. Then, 10.7 kg of iodine was divided into 10 portions, each of which was added at a rate of 500 g per 30 minutes, and the mixture was further stirred for 5 hours. Thereafter, 15 kg of a 16.6% aqueous sodium hydrogen sulfite solution was added dropwise over 30 minutes, and then the mixture was stirred for 1 hour. Further, 18 L of pure water was added dropwise, the mixture was stirred for 1 hour, the precipitate formed was then recovered by filtration, and the filtered product was further subjected to a rinsing treatment with 9 L of pure water. The precipitate recovered was dispersed in 36 L of pure water using another 100 L vessel provided in the same manner, dispersed in water for 1 hour, and then recovered by filtration. The filtered product obtained was subjected to a rinsing treatment with 9 L of pure water three times and then subjected to a rinsing treatment with 9 L of acetonitrile. The filtered product recovered was dispersed in 27 L of acetonitrile in a 100 L reactor vessel provided in the same manner, and the mixture was stirred at room temperature for 1 hour. The precipitate was filtered off by filtration and then subjected to a rinsing treatment using 9 L of acetonitrile. The filtered product recovered was dried at 40° C. for 12 hours to obtain 3.7 kg of 5-iodovanillin which is the target substance.

It was confirmed from the NMR analysis of the target substance obtained that 5-iodovanillin was obtained.

The same iodination step was conducted a plurality of times to obtain 9.0 kg or more of 5-iodovanillin.

### (Step 2): Synthesis of 2-methoxy-4-vinyl-6-iodophenol

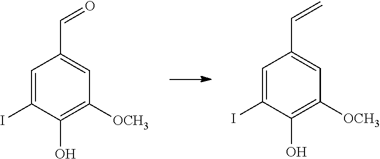

### (Styrenation Step)

A 200 L glass lined reactor vessel and a Teflon-coated TWINSTIR stirring blade were provided, and the reactor vessel was washed with acetone and methanol that is the reaction solvent before the reaction, then filled with a 0.1% aqueous nitric acid solution and washed over 18 hours, and sufficiently washed with pure water. The reactor vessel was further equipped with a condenser. The operation of the solution involving stirring in the reactor vessel was conducted under a nitrogen flow (0.1 L/min).

17.34 kg of methyltriphenylphosphoniumbromide, 1.4 g of methoquinone, and 36 kg of THF were provided and put in the reactor vessel. While the reactor vessel was stirred under a nitrogen flow (0.1 L/min) at 120 rpm, the internal temperature was controlled to −10° C. with a jacket type temperature control mechanism. While the internal temperature was maintained at between −10° C. to 10° C., 8.7 kg of t-BuOK was added in 870 g portions in a stirred state, and after the total amount thereof was added, the mixture was stirred for 30 minutes. Thereafter, while the internal temperature was maintained at between −10° C. to 10° C., 9.0 kg of 5-iodovanillin was added in 900 g portions, and after the total amount thereof was added, the mixture was stirred for 1 hour. Thereafter, the reactor vessel was charged with 58 kg of toluene, and toluene was added to the reaction solution while confirming that the internal temperature was 30° C. or less. Thereafter, the internal temperature was controlled to −5° C. with a jacket type temperature controller. Then, 9.0 kg of 6 M hydrochloric acid was added while stirring. Subsequently, 36 kg of pure water was added, the mixture was stirred while confirming the internal temperature, and continuously cooled to −5° C. until the increase of the internal temperature was settled. After the aqueous phase was discharged, a washing treatment in which 22.5 kg of pure water was added, the mixture was stirred for 10 minutes and then allowed to stand still, and after the interface of two solutions was confirmed, the aqueous phase was discharged, was conducted three times to obtain an organic phase containing toluene and THF as solvents. The organic phase was further distilled off under reduced pressure to obtain a two-time concentrated toluene solution subjected to a concentration treatment.

A 20 L glass lined reactor vessel was provided, and the reactor vessel was washed with acetone and methanol that is the reaction solvent before the reaction, then filled with a 0.1% aqueous nitric acid solution and washed over 18 hours, and sufficiently washed with pure water. After 8.36 kg of THF was put, the reactor vessel was charged with 1.51 kg of concentrated sulfuric acid, while the internal temperature was maintained at 0° C. or less, and the mixture was stirred for 10 minutes to prepare a concentrated sulfuric acid-THF solution.

While the internal temperature of the two-time concentrated toluene solution subjected to a concentration treatment was maintained within a range of −5° C. to 5° C. and stirred at 180 rpm, 9.87 kg of the prepared concentrated sulfuric acid-THF solution was added dropwise, and the mixture was stirred for 1 hour. After the precipitate was confirmed, the mixture was subjected to a filtration treatment to recover the filtrate.

A 50 L glass lined reactor vessel was provided, and the reactor vessel was washed with acetone and methanol that is the reaction solvent before the reaction, then filled with a 0.1% aqueous nitric acid solution and washed over 18 hours, and sufficiently washed with pure water. After 18 L of pure water was put, the total amount of the filtrate recovered by filtration was put therein. After the mixture was stirred for 10 minutes, the aqueous phase was discharged. Washing with 18 L of pure water and discharge of the aqueous phase were additionally carried out two times in the same manner to obtain an organic phase containing toluene and THF as solvents. The organic phase was further distilled off under reduced pressure to obtain a two-time concentrated toluene solution subjected to a concentration treatment.

9 kg of silica gel powder (neutral silica, manufactured by Kanto Chemical Co., Inc.) was added to the obtained toluene solution, and the mixture was stirred for 1 hour. The filtrate was recovered by filtration, and silica was further rinsed with 18 kg of toluene to recover the remaining components.

The obtained toluene solution was subjected to a separation and washing treatment with 18 kg of a 0.1% aqueous NaHSO₃ solution, the recovered toluene phase was then subjected to a separation and washing treatment with 18 kg of a 1% aqueous oxalic acid solution, and the recovered toluene phase was further subjected to a separation and washing treatment with 18 kg of pure water three times. The obtained toluene solution was subjected to a filter treatment using a 10 nm nylon filter (manufactured by KITZ MICROFILTER CORPORATION) to recover the toluene solution. After 51.5 kg of heptane was added to the obtained toluene solution, the toluene solution was cooled with a jacket type temperature control mechanism so that the internal temperature reached −20° C. and allowed to stand still for 5 hours. The precipitate was recovered by filtration and dried at 25° C. to obtain 5.0 kg of 2-methoxy-4-vinyl-6-iodophenol (iodine-containing styrene derivative) that is the target substance.

### (Step 3): Synthesis of 3-Methoxy-4-acetoxy-5-iodostyrene

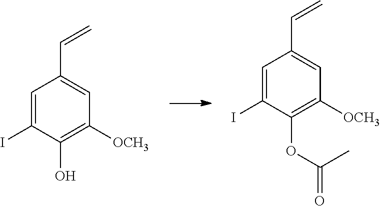

### (Acetyl Protecting Group Introduction Step)

A 200 L glass lined reactor vessel and a Teflon-coated TWINSTIR stirring blade were provided, and the reactor vessel was washed with acetone and methanol that is the reaction solvent before the reaction, then filled with a 0.1% aqueous nitric acid solution and washed over 18 hours, and sufficiently washed with pure water. The reactor vessel was further equipped with a condenser. The operation of the solution involving stirring in the reactor vessel was conducted under a nitrogen flow (0.1 L/min).

5.45 kg of the styrene derivative synthesized in the styrenation step, 49 kg of toluene, and 545 g of silica gel 60 (manufactured by Kanto Chemical Co., Inc.) were put, and the mixture was stirred for 1 hour under a nitrogen flow (0.1 L/min), while the internal temperature was controlled to 20° C. with a jacket type temperature control mechanism. Silica gel 60 was filtered off by filtration, and further, the toluene solution obtained by rinsing silica gel with 2.2 kg of toluene was also recovered.

After the above toluene solution was put in a 200 L reactor vessel provided in the same manner, the internal temperature was controlled to 0° C. with a jacket type temperature control mechanism, and then 5.45 g of methoquinone, 3.0 kg of triethylamine, and 362 g of DMAP were sequentially added in a stirred state, and the mixture was stirred for 30 minutes. Further, 3.0 kg of acetic anhydride was added dropwise while the internal temperature was maintained at 15° C. or less, and the mixture was further stirred for 1 hour. Thereafter, 23.4 kg of 0.2 N hydrochloric acid was added dropwise to the reaction solution in a stirred state while the internal temperature was maintained at 15° C. or less, and the mixture was stirred at 180 rpm for 10 minutes. After the reaction solution was allowed to stand still, the aqueous phase was discharged, 23.4 kg of a 5% by mass aqueous sodium bicarbonate solution was then added so that the internal temperature reached 30° C. or less, the mixture was stirred at 180 rpm for 10 minutes and further allowed to stand still, and the aqueous phase was discharged, whereby a washing treatment was carried out. The washing treatment with the aqueous sodium bicarbonate solution was further conducted once. Then, 15.2 kg of a 3% by mass aqueous oxalic acid solution was added, the mixture was stirred at 180 rpm for 10 minutes and further allowed to stand still, and then the aqueous phase was discharged, whereby a washing treatment with the aqueous oxalic acid solution was carried out. The washing treatment with the aqueous sodium bicarbonate solution was further conducted once. Then, 15.2 kg of pure water was added, the mixture was stirred at 180 rpm for 10 minutes and further allowed to stand still, and then the aqueous phase was discharged, whereby a washing treatment with pure water was carried out. The washing treatment with pure water was further conducted twice. The organic phase subjected to the washing treatment with pure water was further subjected to a treatment with a 10 nm filter (nylon filter, manufactured by KITZ MICROFILTER CORPORATION). The organic phase that was the recovered toluene solution was distilled off under reduced pressure and thereby concentrated so that the concentration was 45% by mass. At this time, the toluene solution was 13.5 kg. Thereafter, the toluene solution was cooled using a jacket type temperature control mechanism so that the internal temperature reached −20° C. Further, after 46.8 kg of heptane cooled to −20° C. was gradually added, the mixture was stirred at 20 rpm for 12 hours while the internal temperature was maintained at −20° C. to −25° C. to form a precipitate. The obtained precipitate was recovered by filtration and air-dried at 20° C. for 24 hours to obtain 4.9 kg of the target substance.

### Example 15

### Synthesis of 5-(1-ethoxyethoxy)-6-methoxy-2-iodostyrene

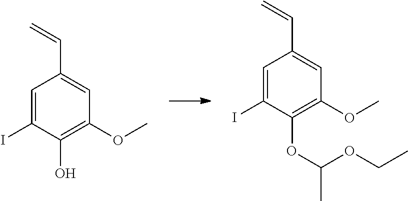 [SMILES: C=Cc1cc(I)c(O)c(OC)c1 ; C=Cc1cc(I)c(OC(C)OCC)c(OC)c1]

In a 1000 mL glass reaction vessel equipped with a reflux tube, 40 g of 2-iodo-6-methoxy-4-vinyl phenol and 360 mL of toluene were put, and the reaction vessel was put in an ice bath under a nitrogen flow so that the internal temperature reached 0° C. Then, 104 g of ethyl vinyl ether and 12.7 g of pyridinium p-toluenesulfonate were put, and the mixture was stirred at 120 rpm for 4 hours. Thereafter, 7.8 mL of triethylamine was added dropwise, 360 mL of distilled water was then put, and the mixture was stirred for 30 minutes. Then, 170 mL of ethyl acetate was put and stirred for 30 minutes, and then the aqueous phase was discharged. Then, 50 g of silica gel 60 N (manufactured by Kanto Chemical Co., Inc.) was put, the mixture was stirred at 50 rpm for 60 minutes, and then the filtrate was recovered by filtration. The obtained filtrate was subjected to a concentration treatment under reduced pressure, subjected to a crystallization treatment in a toluene/hexane solvent system, and further subjected to a rinsing treatment with hexane, and then dried under reduced pressure to obtain 42.93 g of a white solid.

As a result of the analysis by liquid chromatography-mass spectrometry (LC-MS), the molecular weight was found to be 348. The following peaks were found by carrying out 1H-NMR measurement under the above measurement conditions, and the compound was confirmed to have the chemical structure of -5-(2-ethoxyethoxy)-6-methoxy-2-iodostyrene.

δ (ppm) (d6-DMSO): 7.4 (1H, Ph), 7.2 (1H, Ph), 6.7 (1H, —CH═CH2), 5.8 (1H, —CH═CH2), 5.2 (1H, —CH═CH2), 3.8 (3H, —CH3), 5.6 (1H, —CH—), 3.9 (2H, —CH2-), 1.6 (3H, —CH3), 1.2 (3H, —CH3)

### Example 16

### Synthesis of 3-methoxy-4-acetoxy-5-iodostyrene

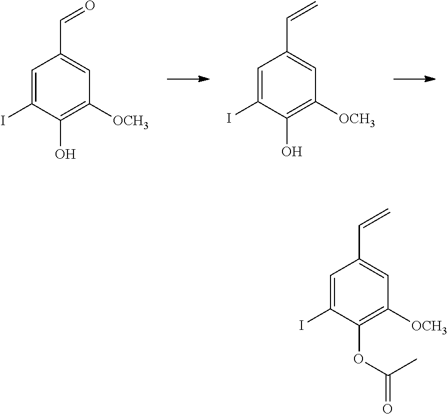

### (Styrenation/Acetylation One-Pot Step)

A 200 L glass lined reactor vessel A and a Teflon-coated TWINSTIR stirring blade were provided, and the reactor vessel was washed with acetone and methanol that is the reaction solvent before the reaction, then filled with a 0.1% aqueous nitric acid solution and washed over 18 hours, and sufficiently washed with pure water. The reactor vessel was further equipped with a condenser. The operation of the solution involving stirring in the reactor vessel was conducted under a nitrogen flow (0.1 L/min).

17.34 kg of methyltriphenylphosphoniumbromide, 1.4 g of methoquinone, and 36 kg of THF were provided and put in the reactor vessel A. While the reactor vessel was stirred under a nitrogen flow (0.1 L/min) at 120 rpm, the internal temperature was controlled to −10° C. with a jacket type temperature control mechanism. While the internal temperature was maintained at between −10° C. to 10° C., 8.7 kg of t-BuOK was added in 870 g portions in a stirred state, and after the total amount thereof was added, the mixture was stirred for 30 minutes. Thereafter, while the internal temperature was maintained at between −10° C. to 10° C., 9.0 kg of 5-iodovanillin obtained in step 1 of Example 14 was added in 900 g portions, and after the total amount thereof was added, the mixture was stirred for 1 hour. Thereafter, the continuous process was conducted, 4960 g of acetic anhydride was added dropwise while confirming that the internal temperature was 15° C. or less, and after the total amount thereof was added, the mixture was stirred for 30 minutes. Subsequently, the reactor vessel was charged with 58 kg of toluene, and toluene was added to the reaction solution while confirming that the internal temperature was 30° C. or less. Thereafter, the internal temperature was controlled to −5° C. with a jacket type temperature controller. Then, 9.0 kg of 6 M hydrochloric acid was added while stirring. Subsequently, 36 kg of pure water was added, the mixture was stirred while confirming the internal temperature, and continuously cooled to −5° C. until the increase of the internal temperature was settled. After the aqueous phase was discharged, an operation in which 38.7 kg of 5% sodium bicarbonate water was added, the mixture was stirred for 10 minutes and then allowed to stand still, and after the interface of two solutions was confirmed, the aqueous phase was discharged, was repeated two times. Subsequently, 22.5 kg of pure water was added, the mixture was stirred for 10 minutes and then allowed to stand still, and a washing treatment in which, after the interface of two solutions was confirmed, the aqueous phase was discharged, was conducted three times to obtain an organic phase containing toluene and THF as solvents. The organic phase was further distilled off under reduced pressure to obtain a two-time concentrated toluene solution subjected to a concentration treatment.

A 20 L glass lined reactor vessel B was provided, and the reactor vessel was washed with acetone and methanol that is the reaction solvent before the reaction, then filled with a 0.1% aqueous nitric acid solution and washed over 18 hours, and sufficiently washed with pure water. After 12.59 kg of THF was put, the reactor vessel was charged with 2.27 kg of concentrated sulfuric acid, while the internal temperature was maintained at 0° C. or less, and the mixture was stirred for 10 minutes to prepare a concentrated sulfuric acid-THF solution.

In the reactor vessel A, while the two-time concentrated toluene solution subjected to a concentration treatment was maintained so that the internal temperature was within a range of −5° C. to 5° C. and stirred at 180 rpm, 9.87 kg of the concentrated sulfuric acid-THF solution separately prepared in the reactor vessel B was added dropwise to the reactor vessel A, and the mixture was stirred for 1 hour. After the precipitate was confirmed, the mixture was subjected to a filtration treatment to recover the filtrate.

A 50 L glass lined reactor vessel C was provided, and the reactor vessel was washed with acetone and methanol that is the reaction solvent before the reaction, then filled with a 0.1% aqueous nitric acid solution and washed over 18 hours, and sufficiently washed with pure water. After 18 L of pure water was put, the total amount of the filtrate recovered by filtration was put therein. After the mixture was stirred for 10 minutes, the aqueous phase was discharged. Washing with 18 L of pure water and discharge of the aqueous phase were additionally carried out two times in the same manner to obtain an organic phase containing toluene and THF as solvents. The organic phase was further distilled off under reduced pressure to obtain a two-time concentrated toluene solution subjected to a concentration treatment.

13.5 kg of silica gel powder (neutral silica, manufactured by Kanto Chemical Co., Inc.) was added to the obtained toluene solution, and the mixture was stirred for 1 hour. The filtrate was recovered by filtration, and silica was further rinsed with 18 kg of toluene to recover the remaining components.

The obtained toluene solution was subjected to a separation and washing treatment with 18 kg of a 0.1% aqueous NaHSO₃ solution, the recovered toluene phase was then subjected to a separation and washing treatment with 18 kg of a 3% aqueous oxalic acid solution, and the recovered toluene phase was further subjected to a separation and washing treatment with 18 kg of pure water three times. The obtained toluene solution was subjected to a filter treatment using a 10 nm nylon filter (manufactured by KITZ MICROFILTER CORPORATION) to recover the toluene solution. After the obtained toluene solution was concentrated, the toluene solution was cooled with a jacket type temperature control mechanism so that the internal temperature reached −20° C., 51.5 kg of heptane was added, and then the mixture was stirred for 20 hours. The precipitate was recovered by filtration and dried at 25° C. to obtain 5.0 kg of 3-methoxy-4-acetoxy-5-iodostyrene (iodine-containing styrene derivative) that is the target substance.

### Example 17

### Synthesis of 2,6-diiodo-4-vinyl phenol

### (Step 1): Synthesis of 3′,5′-diiodo-4′-hydroxyphenyl-1-methoxyethyl

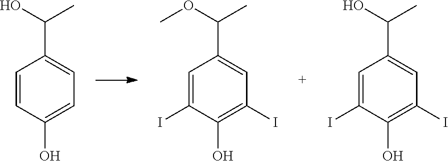

### (Iodination Step)

A 200 L glass lined reactor vessel and a Teflon-coated TWINSTIR stirring blade were provided, and the reactor vessel was washed with acetone and methanol that is the reaction solvent before the reaction, then filled with a 0.1% aqueous nitric acid solution and washed over 18 hours, and sufficiently washed with pure water. The reactor vessel was further equipped with a condenser. The operation of the solution involving stirring in the reactor vessel was conducted under a nitrogen flow (0.1 L/min).

5.55 kg of iodine and 44.4 kg of methanol were put, and the mixture was stirred under a nitrogen flow (0.1 L/min) at room temperature and 180 rpm for 12 hours or more to prepare a solution of iodine in methanol. Then, 3.78 kg of 4-hydroxyphenylethanol and 5.48 kg of pure water were sequentially put while continuously stirring the solution. After the internal temperature was controlled to 0° C. with a jacket type temperature control mechanism, 5.66 kg of a 34% by mass aqueous iodic acid solution was added dropwise so that the internal temperature was within a range of −5° C. to 5° C., the internal temperature was then controlled to 25° C., and the mixture was stirred for 3 hours. After 1.14 kg of a 25% by mass aqueous sodium hydrogen sulfite solution was added, stirring at 180 rpm was performed at 60 rpm. Thereafter, the mixture was cooled while stirring at 180 rpm so that the internal temperature reached 0° C., 56.7 kg of pure water was then added dropwise while the internal temperature was maintained at 0° C., and the mixture was further stirred for 30 minutes. The precipitate formed was filtered off and recovered and then subjected to a rinsing treatment with 1 kg of a 33% by mass aqueous methanol solution. The filtered product obtained was shelf dried at 70° C. to obtain 10.5 kg of 3′,5′-diiodo-4′-hydroxyphenyl-1-methoxyethyl as the target substance.

### (Step 1): Synthesis of 2,6-Diiodo-4-Vinyl Phenol

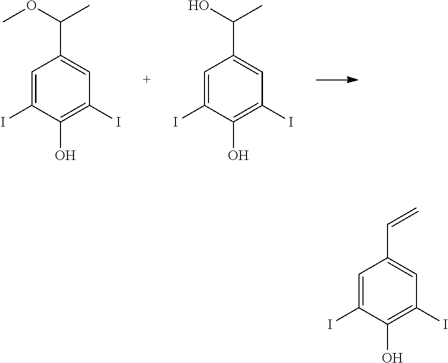

### (Dehydration Reaction Step)

A 200 L glass lined reactor vessel and a Teflon-coated TWINSTIR stirring blade were provided, and the reactor vessel was washed with acetone and methanol that is the reaction solvent before the reaction, then filled with a 0.1% aqueous nitric acid solution and washed over 18 hours, and sufficiently washed with pure water. The reactor vessel was further equipped with a condenser and a Dean-Stark. The operation of the solution involving stirring in the reactor vessel was conducted under a nitrogen flow (0.1 L/min).

10 kg of 3′,5′-diiodo-4′-hydroxyphenyl-1-methoxyethyl synthesized in the iodination step, 1 g of methoquinone, 88 kg of dimethylsulfoxide, and 710 g of methanesulfonic acid were put and dissolved while the mixture was stirred under a nitrogen flow at room temperature and 180 rpm. The mixture was heated so that the internal temperature reached 85° C. with a jacket type temperature control mechanism. 8.7 kg of toluene was added and stirred for 10 minutes, the pressure was further reduced to 3 kPa, and the reduced pressure was maintained until distilling off of toluene with Dean-Stark was settled. The same operation for distilling off toluene was repeated three times. Thereafter, 43.4 kg of toluene was put.

The mixture was cooled using a jacket type temperature control mechanism until the internal temperature reached 25° C. 50 kg of pure water was put, the mixture was stirred at 180 rpm for 10 minutes, then allowed to stand still, and separated into the organic phase and the aqueous phase, and each of them was recovered in a clean drum. The aqueous phase was returned to the reactor vessel, 13 kg of toluene was put, the mixture was stirred at 180 rpm for 10 minutes, then allowed to stand still, and separated into the organic phase and the aqueous phase, and each of them was recovered in a clean drum. The recovered aqueous phase was further subjected to the same extraction operation with 13 kg of toluene twice. The recovered toluene phase was collectively put in a 200 L reactor vessel. Thereafter, 15 kg of a 0.1% by mass aqueous oxalic acid solution was put, the mixture was stirred at an internal temperature of 25° C. for 10 minutes and then allowed to stand still, and then the aqueous phase was discharged. Then, an operation in which 15 kg of pure water was put, the mixture was stirred at an internal temperature of 25° C. for 10 minutes and then allowed to stand still, and then the aqueous phase was discharged, was further repeated three times.

The recovered toluene solution was concentrated by being distilled off under reduced pressure within a range capable of maintaining an internal temperature of 40° C. or less so that 30% by mass of the target substance was obtained, and the concentrated toluene solution was then cooled so that the internal temperature reached 18° C. while stirring at 15 rpm, and continuously stirred for 4 hours. After crystallization of a crystal was confirmed, the solution was cooled at a cooling rate of −1° C./10 minutes so that the internal temperature reached 20° C. Thereafter, 8.3 kg of heptane cooled to −20° C. was gradually added, and then the mixture was stirred for 30 minutes. The precipitated precipitate was filtered off and recovered.

Using a 200 L reactor vessel provided in the same manner, the recovered precipitate and 52 kg of toluene were put and dissolved by being stirred at 25° C. and 120 rpm, and then 17.1 kg of heptane was added. Further, 2 kg of silica gel 60 (manufactured by Kanto Chemical Co., Inc.) was put, and the mixture was stirred for 30 minutes or more. Thereafter, silica put was filtered off and further rinsed with 8.7 kg of toluene, and the filtrate and the rinsing solution were recovered.

Using a 200 L reactor vessel provided in the same manner, the recovered filtrate and the rinsing solution were put therein. Further, 50 g of methoquinone was added, and the mixture was stirred at 180 rpm for 10 minutes. Thereafter, 15 kg of a 0.1% by mass aqueous oxalic acid solution was put, the mixture was stirred at an internal temperature of 25° C. for 10 minutes and then allowed to stand still, and then the aqueous phase was discharged. Then, an operation in which 15 kg of pure water was put, the mixture was stirred at an internal temperature of 25° C. for 10 minutes and then allowed to stand still, and then the aqueous phase was discharged, was further repeated three times.

The recovered toluene solution was concentrated by being distilled off under reduced pressure within a range capable of maintaining an internal temperature of 40° C. or less so that 30% by mass of the target substance was obtained, and the concentrated toluene solution was then cooled so that the internal temperature reached 18° C. while stirring at 15 rpm, and continuously stirred for 4 hours. After crystallization of a crystal was confirmed, the solution was cooled at a cooling rate of −1° C./10 minutes so that the internal temperature reached 20° C. Thereafter, 8.3 kg of heptane cooled to −20° C. was gradually added, and then the mixture was stirred for 30 minutes. The precipitated precipitate was filtered off and recovered.

The recovered precipitate was shelf dried at 25° C. for 72 hours to obtain 4.9 kg of the target substance.

### Synthetic Example AaXX1

### Synthesis of 3,5-dihydroxy-4-iodobenzaldehyde

A 1000 mL glass flask was charged with 55.2 g (0.400 mol) of 3,5-dihydroxybenzaldehyde, 497 mL of methanol, and 16.6 g of activated charcoal, and stirring was initiated under the conditions of room temperature of 20° C. Using a filter loaded with a filter aid, activated charcoal was removed, and the obtained solution was put in a 2000 mL glass flask. Methanol was added until the total weight of the solution reached 1105 g. Subsequently, blowing of nitrogen at a flow rate of 10 mL/minute was initiated. After 38.6 g (0.152 mol) of iodine was added, 134 g (0.0760 mol) of an aqueous iodic acid solution with a mass % concentration of 10 was added dropwise, while the temperature was further regulated to 30° C. or less. After the mixture was stirred for 3 hours, 8 g of an aqueous sodium hydrogen sulfite solution with a mass percent concentration of 5 was added. The pressure in the container was reduced to 35 hPa, and the container was immersed in a water bath at 40° C. to concentrate the solution to dryness, whereby 166 g of a solid was obtained. The obtained solid was suspended and stirred with 780 g of ion exchanged water, and the crude was recovered by suction filtration. The crude was dried in vacuo at 60° C. to obtain 79.2 g of a white solid. The yield was 75%.

As a result of the analysis by liquid chromatography-mass spectrometry (LC-MS), the molecular weight was found to be 264.

The following peaks were found by carrying out ¹H-NMR measurement under the aforementioned measurement conditions, and the compound was confirmed to have the chemical structure of 3,5-dihydroxy-4-iodobenzaldehyde.

δ (ppm) (d6-DMSO): 10.7 (2H, —OH), 9.2 (1H, —CHO), 6.8 (2H, Ph)

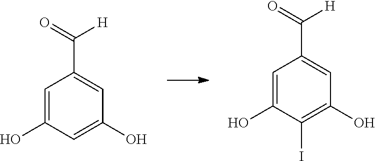 [SMILES: O=Cc1cc(O)cc(O)c1 ; O=Cc1cc(O)c(I)c(O)c1]

### Synthetic Example AaXX2

### Synthesis of 3,5-diacetoxy-4-iodobenzaldehyde

A 1000 mL glass flask as a reaction vessel was charged with 75.0 g (0.284 mol) of 3,5-dihydroxy-4-iodobenzaldehyde obtained in Synthetic Example AaXX1, 759 mL of ethyl acetate, 60.3 g (0.597 mol) of triethylamine, and 3.82 g (0.0312 mol) of 4-dimethylaminopyridine, and stirring was initiated at 0° C. Subsequently, blowing of nitrogen at a flow rate of 10 mL/minute was initiated. While the temperature was regulated to 10° C. or less, 60.9 g (0.597 mol) of acetic anhydride was added dropwise, and the mixture was stirred at room temperature of 20° C. for 2 hours. After no remaining of the hydroxyl group of 3,5-dihydroxy-4-iodobenzaldehyde was confirmed by liquid chromatography, 169 g of 0.2 M hydrochloric acid was added to separate the solution. The organic layer was sequentially washed with ion exchanged water, a 5% by mass sodium bicarbonate water solution, and ion exchanged water. After the organic layer was dehydrated with sodium sulfate, the pressure of the container was reduced to 90 hPa, the container was immersed in a water bath at 30° C. to concentrate the solution to dryness, whereby the crude was recovered. The obtained crude was purified by recrystallization with toluene and then dried in vacuo at 45° C. to obtain 63.7 g of a white solid. The yield was 65%.

As a result of the analysis by liquid chromatography-mass spectrometry (LC-MS), the molecular weight was found to be 348.

The following peaks were found by carrying out ¹H-NMR measurement under the aforementioned measurement conditions, and the compound was confirmed to have the chemical structure of 3,5-diacetoxy-4-iodobenzaldehyde.

δ (ppm) (d6-DMSO): 10.0 (1H, —CHO), 7.6 (2H, Ph), 2.4 (6H, —CH3)

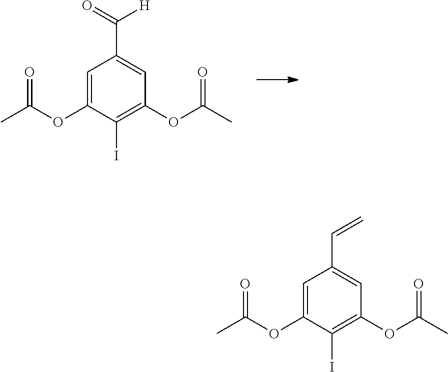 [SMILES: CC(=O)Oc1cc(C=O)cc(OC(C)=O)c1I ; C=Cc1cc(OC(C)=O)c(I)c(OC(C)=O)c1]

### Synthetic Example AaXX3

### Synthesis of 3,5-diacetoxy-4-iodostyrene

A 1000 mL glass reaction vessel was charged with 61.6 g (0.172 mol) of methyltriphenylphosphoniumbromide and 450 mL of THF, and blowing of nitrogen into the reaction vessel at a flow rate of 10 mL/min and stirring were initiated. The reaction vessel was immersed in ice water and cooled to 0° C., and charged with 19.3 g (0.172 mol) of potassium tert-butoxide by portionwise addition, and stirring was continued for 30 minutes. The reaction vessel was charged with 40.0 g (0.115 mol) of 3,5-diacetoxy-4-iodobenzaldehyde obtained from the aforementioned Synthetic Example AaXX2 by portionwise addition and stirred for 1 hour, and then the reaction solution was gradually added to 500 g of 3 M hydrochloric acid put in another glass reaction vessel. The reaction vessel was returned to room temperature, 750 mL of toluene was put therein, and the mixture was stirred. The organic layer was transferred to a separating funnel and washed with 3 M hydrochloric acid and ion exchanged water a plurality of times to obtain a Wittig reaction solution containing 3,5-diacetoxy-4-iodostyrene (compound XX) and triphenylphosphine oxide.

The obtained Wittig reaction solution was purified by column chromatography, and the obtained solid of the compound XX was dried in vacuo at 25° C. to obtain 10.7 g of a white solid. The yield was 27%.

As a result of the analysis by liquid chromatography-mass spectrometry (LC-MS), the molecular weight was found to be 346.

The following peaks were found by carrying out ¹H-NMR measurement under the aforementioned measurement conditions, and the compound was confirmed to have the chemical structure of 3,5-diacetoxy-4-iodostyrene (compound XX3) given below.

δ (ppm) (d6-DMSO): 7.3 (1H, Ph), 6.7 (1H, —CH═CH2), 6.0 (1H, —CH═CH2), 5.4 (1H, —CH═CH2), 2.3 (6H, —CH3)

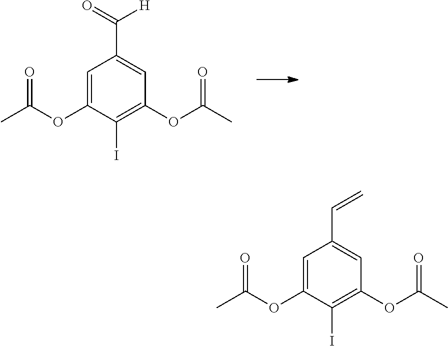 [SMILES: CC(=O)Oc1cc(C=O)cc(OC(C)=O)c1I ; C=Cc1cc(OC(C)=O)c(I)c(OC(C)=O)c1]

### Example B1

### Synthesis of compound Ma1a (removal of phosphine oxide using sulfuric acid)

The compound Ma1a was synthesized by steps 1 to 3.

### (Step 1): Iodination

4-Hydroxy-5-iodo-3-methoxybenzaldehyde was obtained by the same operation as in Example 1 (step 1).

### (Step 2): Acetyl Protection

4-Acetoxy-5-iodo-3-methoxybenzaldehyde was obtained by the same operation as in Example 2 (step 2).

### (Step 3): Olefination and Removal of Phosphine Oxide by Acid Addition Method

32.9 g of the compound Ma1a was obtained by the method described in Example 2 (step 3), except that the temperature in the reaction between the Wittig reaction solution and the sulfuric acid solution was changed to −20° C. instead of 0° C. The LC purity at 254 nm was 99.9%, and the GPC purity was 99.9%.

### Example B2

### Synthesis of compound Ma1a (removal of phosphine oxide using sulfuric acid)

The compound Ma1a was synthesized by steps 1 to 3.

### (Step 1): Iodination

4-Hydroxy-5-iodo-3-methoxybenzaldehyde was obtained by the same operation as in Example 1 (step 1).

### (Step 2): Acetyl Protection

4-Acetoxy-5-iodo-3-methoxybenzaldehyde was obtained by the same operation as in Example 2 (step 2).

### (Step 3): Olefination and Removal of Phosphine Oxide by Acid Addition Method

31.9 g of the compound Ma1a was obtained by the method described in Example 2 (step 3), except that the temperature in the reaction between the Wittig reaction solution and the sulfuric acid solution was changed to 30° C. instead of 0° C. The LC purity at 254 nm was 99.8%, and the GPC purity was 99.7%.

### Example B3

### Synthesis of compound Ma1a (removal of phosphine oxide using sulfuric acid)

The compound Ma1a was synthesized by steps 1 to 3.

### (Step 1): Iodination

4-Hydroxy-5-iodo-3-methoxybenzaldehyde was obtained by the same operation as in Example 1 (step 1).

### (Step 2): Acetyl Protection

4-Acetoxy-5-iodo-3-methoxybenzaldehyde was obtained by the same operation as in Example 2 (step 2).

### (Step 3): Olefination and Removal of Phosphine Oxide by Acid Addition Method

33.5 g of the compound Ma1a was obtained by the method described in Example 2 (step 3), except for using 144 mL of 1-butanol instead of 144 mL of toluene as an extraction solvent. The LC purity at 254 nm was 99.4%, and the GPC purity was 99.9%.

### Example B4

### Synthesis of compound Ma1a (removal of phosphine oxide using sulfuric acid)

The compound Ma1a was synthesized by steps 1 to 3.

### (Step 1): Iodination

4-Hydroxy-5-iodo-3-methoxybenzaldehyde was obtained by the same operation as in Example 1 (step 1).

### (Step 2): Acetyl Protection

4-Acetoxy-5-iodo-3-methoxybenzaldehyde was obtained by the same operation as in Example 2 (step 2).

### (Step 3): Olefination and Removal of Phosphine Oxide by Acid Addition Method

33.6 g of the compound Ma1 was obtained by the method described in Example 2 (step 3), except for using 18.4 g of sulfuric acid and 104 mL of THF instead of the sulfuric acid solution of 7.80 g of sulfuric acid and 44.2 mL of THF. The LC purity at 254 nm was 99.9%, and the GPC purity was 99.9%.

### Example B5

### Synthesis of compound Ma2 (removal of phosphine oxide using sulfuric acid)

The compound Ma2 was synthesized by steps 1 to 2.

### (Step 1): Iodination

4-Hydroxy-3,5-diiodo-benzaldehyde was obtained by the same operation as in Example 10 (step 1).

### (Step 2): Olefination and Removal of Phosphine Oxide by Addition of Acid (Synthesis of Compound Ma2)

63.8 g of the compound Ma2 was obtained by the method described in Example 10 (step 2), except for using 13.0 g of sulfuric acid and 13.0 mL of THF instead of the sulfuric acid solution of 13.0 g of sulfuric acid and 82.8 mL of THF. The LC purity at 254 nm was 99.9%, and the GPC purity was 99.9%.

### Example B6

### Synthesis of compound Ma2 (removal of phosphine oxide using sulfuric acid)

The compound Ma2 was synthesized by steps 1 to 2.

### (Step 1): Iodination

4-Hydroxy-3,5-diiodo-benzaldehyde was obtained by the same operation as in Example 10 (step 1).

### (Step 2): Olefination and Removal of Phosphine Oxide by Addition of Acid (Synthesis of Compound Ma2)

62.7 g of the compound Ma2 was obtained by the method described in Example 10 (step 2), except for using 13.0 g of sulfuric acid and 13.0 mL of 1,4-dioxane instead of the sulfuric acid solution of 13.0 g of sulfuric acid and 82.8 mL of THF. The LC purity at 254 nm was 99.9%, and the GPC purity was 99.9%.

### Example B7

### Synthesis of compound Ma2 (removal of phosphine oxide using sulfuric acid)

The compound Ma2 was synthesized by steps 1 to 2.

### (Step 1): Iodination

4-Hydroxy-3,5-diiodo-benzaldehyde was obtained by the same operation as in Example 10 (step 1).

### (Step 2): Olefination and Removal of Phosphine Oxide by Addition of Acid (Synthesis of Compound Ma2)

64.0 g of the compound Ma2 was obtained by the method described in Example 10 (step 2), except for using 43.9 g of p-toluenesulfonic acid and 102 mL of methanol instead of the sulfuric acid solution of 13.0 g of sulfuric acid and 82.8 mL of THF. The LC purity at 254 nm was 99.8%, and the GPC purity was 99.9%.

### Example B8

### Synthesis of compound Ma1

The compound Ma1 was synthesized by the following steps 1 to 2.

### (Step 1): Iodination

4-Hydroxy-5-iodo-3-methoxybenzaldehyde was obtained by the same operation as in Example 1 (step 1).

### (Step 2): Olefination and Removal of Phosphine Oxide by Addition of Acid (Synthesis of Compound Ma2)

A 1000 mL glass reaction vessel was charged with 134 g (0.375 mol) of methyltriphenylphosphoniumbromide and 348 mL of THF, and blowing of nitrogen into the reaction vessel at a flow rate of 10 mL/min and stirring were initiated. The reaction vessel was immersed in ice water and cooled to 0° C., and charged with 95.2 g (0.625 mol) of diazabicycloundecene by portionwise addition, and stirring was continued for 30 minutes. The reaction vessel was charged with 69.5 g (0.250 mol) of 4-hydroxy-5-iodo-3-methoxybenzaldehyde obtained in step 1 by portionwise addition, the temperature thereof was increased to room temperature of 25° C., and the mixture was stirred for 6 hours, the reaction solution was then gradually added to 126 g of 3 M hydrochloric acid put in another glass reaction vessel, 209 mL of toluene was put therein, and the mixture was stirred. The organic layer was transferred to a separating funnel and washed with 3 M hydrochloric acid and ion exchanged water a plurality of times to obtain a Wittig reaction solution containing the compound Ma1 and triphenylphosphine oxide.

The subsequent steps were carried out by the same operation as in Example 1 (step 2) to obtain 46.0 g of the compound Ma1. The LC purity at 254 nm was 99.9%, and the GPC purity was 99.9%.

The disclosure of Japanese Patent Application No. 2022-203721 filed on Dec. 20, 2022, and the disclosure of Japanese Patent Application No. 2023-182078 filed on Oct. 23, 2023 are incorporated herein by reference in its entirety. All literatures, patent applications, and technical standards described herein are incorporated herein by reference to the same extent as if each individual literature, patent application, or technical standard is specifically and individually indicated to be incorporated by reference.

1. A method for producing a compound A represented by the following formula (1): 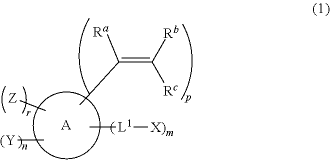
wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a urethane group, a urea group, an amide group, or an imide group, and the ether group, the ester group, the thioether group, the amino group, the thioester group, the acetal group, the urethane group, the urea group, the amide group, and the imide group of L¹ optionally have a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 18 carbon atoms and optionally having a substituent;
A is an organic group having 6 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
p is an integer of 1 or more, m is an integer of 1 or more, n is an integer of 0 or more, and r is an integer of 0 or more;
the method comprising at least one of step HX, step ST, and step PR:
step HX: a step of introducing a halogen or a group containing a halogen,
step ST: a step of introducing an unsaturated double bond, and
step PR: a step of introducing a group represented by the following formula (Y-0): 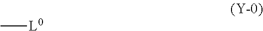
wherein each L⁰ is independently one or more groups selected from the group consisting of an alkoxy group, an ester group, an ether group, a thioether group, an acetal group, a thioacetal group, a carboxyalkoxy group, a carbonate ester group, a sulfonyl group, and a silyl group, or a hydrolyzable group.

2. The production method according to claim 1, wherein the step ST comprises any one of step (W), step (C), and step (D):
step (W): a step comprising at least
W1) a step of providing a carbonyl compound B represented by the following formula (3a) or the following formula (3b), and
W2) a step of forming an unsaturated double bond from the carbonyl moiety of the carbonyl compound B: 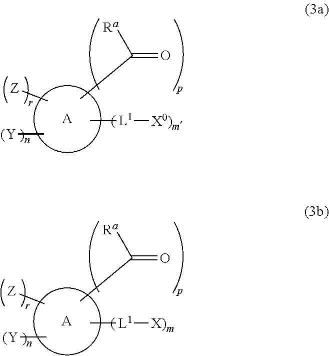
wherein
X, L¹, Y, R^{a}, Z, A, m, n, p, and r are as defined in the formula (1);
X⁰ is H or an organic group having 1 to 30 carbon atoms; and
m′ is an integer of 0 or more,
step (C): a step comprising at least
C1) a step of providing the carbonyl compound B,
C2) a step of obtaining a compound represented by the following formula (SA2a) or the following formula (SA2b) by using the carbonyl compound B and a compound represented by the formula (RM1) or malononitrile, and
C3) a step of obtaining a compound represented by the following formula (0a) or the following formula (0b) by using the compound represented by the following formula (SA2a) or the following formula (SA2b) and a fluoride source: 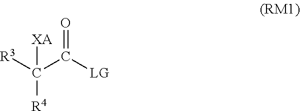
wherein
LG is a group selected from a hydroxy group, an alkoxy group, a carbonate ester group, an acetal group, and a carboxyl group, and the alkoxy group, the carbonate ester group, the acetal group, and the carboxyl group contain an aliphatic group or aromatic group having 1 to 60 carbon atoms and optionally having a substituent;
R³ is a hydrogen group, or a carboxyl group or ester group having 1 to 60 carbon atoms and optionally having a substituent;
R⁴ is a hydrogen group; and
XA is a group selected from a hydrogen group and a halogen group, 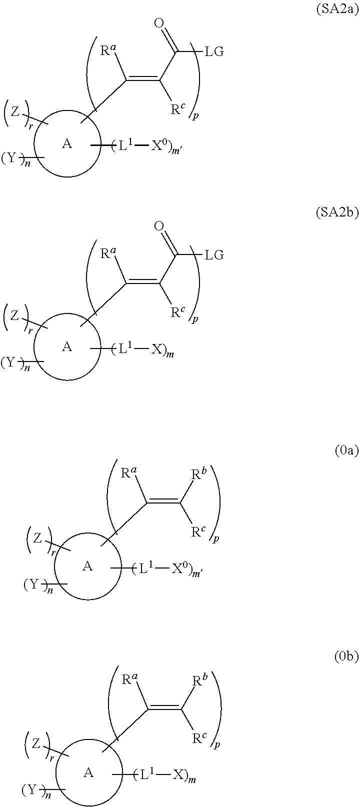
wherein
X, X⁰, L¹, Y, A, Z, p, m, m′, n, r, R^{a}, R^{b}, R^{c}, and LG are as defined in the formula (1), the formula (3a), the formula (3b), and the formula (RM1), and
step (D): a step comprising at least
D1) a step of providing a compound represented by the formula (1-1a) or the formula (1-1b): 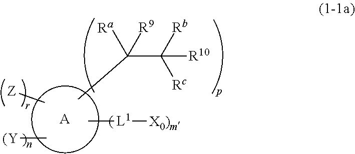
wherein
X₀, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{D} is an organic group having 1 to 30 carbon atoms and optionally having a substituent, 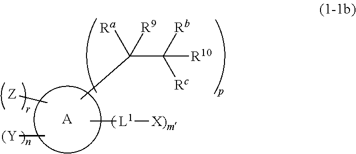
wherein
X, L¹, Y, A, Z, p, m′, n, r, R^{a}, R^{b}, and R^{c} are as defined in the formula (1), the formula (3a), and the formula (3b); and
each of R⁹ to R¹⁰ is independently H, OH, OCH₃, OR^{D}, a halogen, a cyano group, or an organic group having 1 to 8 carbon atoms and optionally having a substituent, and at least one of R⁹ to R¹⁰ is OH, OCH₃, or OR^{D}, provided that R^{D} is an organic group having 1 to 30 carbon atoms and optionally having a substituent, and
D2) a step of obtaining a compound represented by the following formula (0a) or the following formula (0b) from the compound represented by the formula (1-1a) or the formula (1-1b) by a dehydration reaction using an acid as a catalyst: 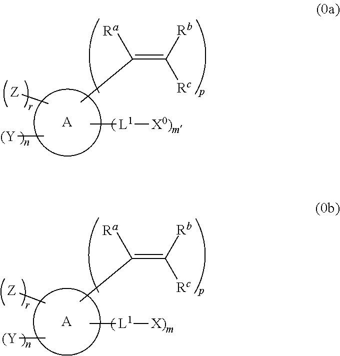
wherein
X, X⁰, L¹, Y, A, Z, p, m, m′, n, r, R^{a}, R^{b}, and R^{c} are as defined in the formula (1), the formula (3a), and the formula (3b).

3. The production method according to claim 2, wherein an organic phosphorus compound is used in step W2.

4. The production method according to claim 3, further comprising a step of solidifying phosphine oxide generated and removing the phosphine oxide by solid-liquid separation in step W2.

5. The production method according to claim 4, wherein an acid is added to solidify the phosphine oxide as a composite with the acid.

6. The production method according to claim 5, wherein amounts of substance of the acid and the phosphine oxide satisfy the following expression:
$$0.5 \leq \frac{\sum ( Amount of substance of the acid ) \times ( Valence of the acid )}{( Amount of substance of the phosphine oxide )} \leq 5$$

7. The production method according to claim 4, wherein the phosphine oxide is solidified by changing a solvent system.

8. The production method according to claim 3, wherein an organic base containing no alkali metal as a constituent element is further used in step W2.

9. The production method according to claim 7, wherein
the carbonyl compound B is a compound represented by the following formula (4a) or the following formula (4b), and
an HSP distance between a compound represented by the following formula (5a) or the following formula (5b) formed by using an organic phosphorus compound in step W2 and the phosphine oxide is 6.5 or more: 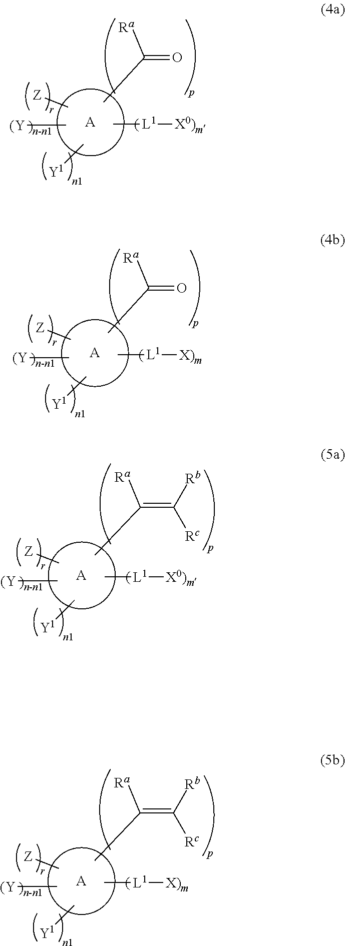
wherein
X, X⁰, L¹, Y, R^{a}, R^{b}, R^{c}, Z, A, m, m′, n, p, and r are as defined in the formula (1), the formula (3a), and the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally has a substituent; and
n1 is an integer of 1 or more and n or less.

10. The production method according to claim 9, wherein the step HX further comprises a step of halogenating the compound represented by the formula (3a) to produce the compound represented by the formula (3b), a step of halogenating the compound represented by the formula (0a) to produce the compound represented by the formula (0b), and a step of halogenating the compound represented by the formula (4a) to produce the compound represented by the formula (4b).

11. The production method according to claim 2, wherein one or more solvents selected from the group consisting of γbutyrolactone, dimethylformamide, dioxane, cyclopentyl methyl ether, toluene, and diglyme are further used in step C3.

12. The production method according to claim 2, wherein one or more solvents selected from the group consisting of polar aprotic solvents having a relative permittivity at 20° C. to 30° C. of 20 or more, and one or more solvents selected from the group consisting of ethanol, propanol, isopropyl alcohol, butanol, isobutanol, sec-butanol, tert-butanol, 1,2-dimethoxyethane, diisopropyl ether, ethyl acetate, tetrahydrofuran, dioxane, methyl ethyl ketone, carbon tetrachloride, chloroform, dichloroethane, benzene, toluene, o-xylene, cyclohexane, hexane, acetonitrile, nitromethane, and pyridine are further used in step D2.

13. The production method according to claim 1, further comprising a step of removing impurities using an adsorbent.

14. The production method according to claim 1, further comprising a step of removing impurities by a reslurry treatment.

15. The production method according to claim 2, comprising step STPR of carrying out one step of step ST and step PR and then continuously carrying out the other step without carrying out isolation and purification operation.

16. The production method according to claim 15, wherein step ST is step (W).

17. The production method according to claim 16, wherein an organic phosphorus compound is used in step W2.

18. The production method according to claim 17, further comprising a step of solidifying phosphine oxide generated and removing the phosphine oxide by solid-liquid separation in step STPR.

19. The production method according to claim 18, wherein an acid is added to solidify the phosphine oxide as a composite with the acid.

20. The production method according to claim 19, wherein amounts of substance of the acid and the phosphine oxide satisfy the following expression.
$$0.5 \leq \frac{\sum ( Amount of substance of the acid ) \times ( Valence of the acid )}{( Amount of substance of the phosphine oxide )} \leq 5$$

21. The production method according to claim 15, wherein step ST and step PR are carried out in the same reaction vessel.

22. The production method according to claim 15, wherein one step is carried out and the other step is then continuously carried out without exchanging a solvent in step STPR.

23. (canceled)

24. The method for producing a compound represented by the formula (3b) according to claim 9, wherein the compound A represented by the formula (1) is a compound represented by the formula (3b), and the method comprises either step HX or step PR.

25.-26. (canceled)

27. The production method according to claim 24, wherein the compound represented by the formula (3b) is represented by the following formula (4b): 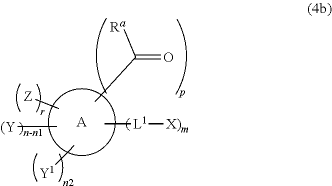
wherein
X, L¹, Y, R^{a}, Z, A, m, n, p, and r are as defined in the formula (3b);
each Y¹ is independently an alkoxy group, an ester group, an acetal group, a carboxyalkoxy group, a carbonate ester group, an ether group, or a thioether group, and the alkoxy group, the ester group, the acetal group, the carboxyalkoxy group, the carbonate ester group, the ether group, and the thioether group of Y¹ optionally have a substituent; and
n1 is an integer of 1 or more and n or less.

28. The production method according to claim 24, comprising a step of removing impurities using an adsorbent.

29. The production method according to claim 24, further comprising a step of removing impurities by a reslurry treatment.

30.-33. (canceled)
